(19) United States
(12) Patent Application Publication
(10) Pub. No.: US 2015/0259710 A1
(43) Pub. Date: Sep. 17, 2015

(54) DECARBOXYLASE PROTEINS WITH HIGH KETO-ISOVALERATE DECARBOXYLASE ACTIVITY

(71) Applicants: Catherine Asleson Dundon, Englewood, CO (US); Kevin Roberg-Perez, Englewood, CO (US); Christopher Snow, Ft. Collins, CO (US); Peter Meinhold, Denver, CO (US)

(72) Inventors: Catherine Asleson Dundon, Englewood, CO (US); Kevin Roberg-Perez, Englewood, CO (US); Christopher Snow, Ft. Collins, CO (US); Peter Meinhold, Denver, CO (US)

(73) Assignee: Gevo, Inc., Englewood, CO (US)

(21) Appl. No.: 13/561,560

(22) Filed: Jul. 30, 2012

Related U.S. Application Data
(60) Provisional application No. 61/512,810, filed on Jul. 28, 2011.

(51) Int. Cl. C12P 7/16; C12N 9/04; C12N 9/88

(52) U.S. Cl. CPC C12P 7/16; C12N 9/88; C12N 9/0006; C12Y 101/01001; C12Y 101/01086; C12Y 401/01

(57) ABSTRACT

The present invention relates to recombinant microorganisms comprising an isobutanol producing metabolic pathway and methods of using said recombinant microorganisms to produce isobutanol. In various aspects of the invention, the recombinant microorganisms may comprise at least one nucleic acid molecule encoding a polypeptide with keto-isovalerate decarboxylase (KIVD) activity, wherein said polypeptide is at least about 65%, 70%, 75%, 80%, 85%, 90%, 91%, 92%, 93%, 94%, 95%, 96%, 97%, 98%, or 99% identical to a polypeptide selected from SEQ ID NOs: 1-214. Also provided are modified decarboxylases exhibiting an improved ability to utilize α-ketoisovalerate as a substrate in various beneficial enzymatic conversions.

### CROSS REFERENCE TO RELATED APPLICATIONS

This application claims priority to U.S. Provisional Application Ser. No. 61/512,810, filed Jul. 28, 2011, which is herein incorporated by reference in its entirety for all purposes.

### TECHNICAL FIELD

Recombinant microorganisms and methods of producing such microorganisms are provided. Also provided are methods of producing beneficial metabolites including fuels and chemicals by contacting a suitable substrate with the recombinant microorganisms of the invention and enzymatic preparations therefrom.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: GEVO_{—}066_{—}01US_SeqList_ST25.txt, date recorded: Jul. 27, 2012, file size: 1,137 kilobytes).

### BACKGROUND

The ability of microorganisms to convert sugars to beneficial metabolites including fuels, chemicals, and amino acids has been widely described in the literature in recent years. See, e.g., Alper et al., 2009, Nature Microbiol. Rev. 7: 715-723 and McCourt et al., 2006, Amino Acids 31: 173-210. Recombinant engineering techniques have enabled the creation of microorganisms that express biosynthetic pathways capable of producing a number of useful products, including the commodity chemical, isobutanol.

Isobutanol, also a promising biofuel candidate, has been produced in recombinant microorganisms expressing a heterologous, five-step metabolic pathway (See, e.g., WO/2007/050671 to Donaldson et al., WO/2008/098227 to Liao et al., and WO/2009/103533 to Festel et al.). However, the microorganisms produced to date have fallen short of commercial relevance due to their low performance characteristics, including, for example low productivities, low titers, and low yields.

The fourth step of the isobutanol producing metabolic pathway is catalyzed by keto-isovalerate decarboxylase (KIVD), which converts alpha-ketoisovalerate to isobutyraldehyde. Because KIVD is an essential enzyme in the isobutanol production pathway, it is desirable that recombinant microorganisms engineered to produce isobutanol exhibit optimal KIVD activity. The present application addresses this need by identifying several enzymes that exhibit high activity for the conversion of alpha-ketoisovalerate to isobutyraldehyde within an isobutanol production pathway. Moreover, the enzymes identified herein have low activity using pyruvate, thereby reducing the conversion of pyruvate to the unwanted by-product ethanol in recombinant isobutanol producing microorganisms. Accordingly, this application describes methods of increasing isobutanol production through the use of recombinant microorganisms comprising enzymes with improved properties for the production of isobutanol.

### SUMMARY OF THE INVENTION

The present inventors have discovered a group of enzymes with high level activity for the conversion of alpha-ketoisovalerate to isobutyraldehyde in the isobutanol pathway. The use of one or more of these enzymes can improve production of the isobutanol in recombinant microorganisms expressing an engineered isobutanol producing metabolic pathway.

In a first aspect, the application relates to a recombinant microorganism comprising at least one nucleic acid molecule encoding a polypeptide with keto-isovalerate decarboxylase (KIVD) activity, wherein said polypeptide is at least about 65% identical to a polypeptide selected from SEQ ID NOs: 1-4. In one embodiment, the polypeptide with keto-isovalerate decarboxylase (KIVD) activity is derived from the genus Lactococcus. In a specific embodiment, the polypeptide with keto-isovalerate decarboxylase (KIVD) activity is derived from Lactococcus lactis.

In another aspect, the application relates to a recombinant microorganism comprising at least one nucleic acid molecule encoding a polypeptide with keto-isovalerate decarboxylase (KIVD) activity, wherein said polypeptide is at least about 65% identical to SEQ ID NO: 5. In one embodiment, the polypeptide with keto-isovalerate decarboxylase (KIVD) activity is derived from the genus Melissococcus. In a specific embodiment, the polypeptide with keto-isovalerate decarboxylase (KIVD) activity is derived from Melissococcus plutonius.

In yet another aspect, the application relates to a recombinant microorganism comprising at least one nucleic acid molecule encoding a polypeptide with keto-isovalerate decarboxylase (KIVD) activity, wherein said polypeptide is at least about 65% identical to SEQ ID NO: 6. In one embodiment, the polypeptide with keto-isovalerate decarboxylase (KIVD) activity is derived from the genus Listeria. In a specific embodiment, the polypeptide with keto-isovalerate decarboxylase (KIVD) activity is derived from Listeria grayi.

In yet another aspect, the application relates to a recombinant microorganism comprising at least one nucleic acid molecule encoding a polypeptide with keto-isovalerate decarboxylase (KIVD) activity, wherein said polypeptide is at least about 65% identical to a polypeptide selected from SEQ ID NOs: 7-44. In one embodiment, the polypeptide with keto-isovalerate decarboxylase (KIVD) activity is derived from a genus selected from Staphylococcus or Macrococcus. In a specific embodiment, the polypeptide with keto-isovalerate decarboxylase (KIVD) activity is derived from Staphylococcus aureus, Staphylococcus epidermidis, Staphylococcus capitis, Staphylococcus haemolyticus, Staphylococcus warneri, Staphylococcus caprae, Staphylococcus saprophyticus, Staphylococcus hominis, Staphylococcus carnosus, Staphylococcus lugdunensis, or Macrococcus caseolyticus.

In yet another aspect, the application relates to a recombinant microorganism comprising at least one nucleic acid molecule encoding a polypeptide with keto-isovalerate decarboxylase (KIVD) activity, wherein said polypeptide is at least about 65% identical to a polypeptide selected from SEQ ID NOs: 45-46. In one embodiment, the polypeptide with keto-isovalerate decarboxylase (KIVD) activity is derived from the genus Staphylococcus. In a specific embodiment, the polypeptide with keto-isovalerate decarboxylase (KIVD) activity is derived from Staphylococcus pseudintermedius.

In yet another aspect, the application relates to a recombinant microorganism comprising at least one nucleic acid molecule encoding a polypeptide with keto-isovalerate decarboxylase (KIVD) activity, wherein said polypeptide is at least about 65% identical to a polypeptide selected from SEQ ID NOs: 47-48. In one embodiment, the polypeptide with keto-isovalerate decarboxylase (KIVD) activity is derived from a genus selected from Bacillus or Clostridium. In a specific embodiment, the polypeptide with keto-isovalerate decarboxylase (KIVD) activity is derived from Bacillus cereus or Clostridium acetobutylicum.

In yet another aspect, the application relates to a recombinant microorganism comprising at least one nucleic acid molecule encoding a polypeptide with keto-isovalerate decarboxylase (KIVD) activity, wherein said polypeptide is at least about 65% identical to a polypeptide selected from SEQ ID NOs: 49-90. In one embodiment, the polypeptide with keto-isovalerate decarboxylase (KIVD) activity is derived from the genus Bacillus. In a specific embodiment, the polypeptide with keto-isovalerate decarboxylase (KIVD) activity is derived from Bacillus anthracis, Bacillus cereus, or Bacillus thuringiensis.

In yet another aspect, the application relates to a recombinant microorganism comprising at least one nucleic acid molecule encoding a polypeptide with keto-isovalerate decarboxylase (KIVD) activity, wherein said polypeptide is at least about 65% identical to a polypeptide selected from SEQ ID NOs: 91-92. In one embodiment, the polypeptide with keto-isovalerate decarboxylase (KIVD) activity is derived from the genus Helicobacter. In a specific embodiment, the polypeptide with keto-isovalerate decarboxylase (KIVD) activity is derived from Helicobacter felis or Helicobacter mustelae.

In yet another aspect, the application relates to a recombinant microorganism comprising at least one nucleic acid molecule encoding a polypeptide with keto-isovalerate decarboxylase (KIVD) activity, wherein said polypeptide is at least about 65% identical to SEQ ID NO: 93. In one embodiment, the polypeptide with keto-isovalerate decarboxylase (KIVD) activity is derived from the genus Sarcina. In a specific embodiment, the polypeptide with keto-isovalerate decarboxylase (KIVD) activity is derived from Sarcina ventriculi.

In yet another aspect, the application relates to a recombinant microorganism comprising at least one nucleic acid molecule encoding a polypeptide with keto-isovalerate decarboxylase (KIVD) activity, wherein said polypeptide is at least about 65% identical to SEQ ID NO: 94. In one embodiment, the polypeptide with keto-isovalerate decarboxylase (KIVD) activity is derived from the genus Nostoc. In a specific embodiment, the polypeptide with keto-isovalerate decarboxylase (KIVD) activity is derived from Nostoc punctiforme.

In yet another aspect, the application relates to a recombinant microorganism comprising at least one nucleic acid molecule encoding a polypeptide with keto-isovalerate decarboxylase (KIVD) activity, wherein said polypeptide is at least about 65% identical to SEQ ID NO: 95. In one embodiment, the polypeptide with keto-isovalerate decarboxylase (KIVD) activity is derived from the genus Salinispora. In a specific embodiment, the polypeptide with keto-isovalerate decarboxylase (KIVD) activity is derived from Salinispora arenicola.

In yet another aspect, the application relates to a recombinant microorganism comprising at least one nucleic acid molecule encoding a polypeptide with keto-isovalerate decarboxylase (KIVD) activity, wherein said polypeptide is at least about 65% identical to a polypeptide selected from SEQ ID NOs: 96-100. In one embodiment, the polypeptide with keto-isovalerate decarboxylase (KIVD) activity is derived from the genus Leishmania. In a specific embodiment, the polypeptide with keto-isovalerate decarboxylase (KIVD) activity is derived from Leishmania mexicana, Leishmania major, Leishmania braziliensis, Leishmania donovani, or Leishmania infantum.

In yet another aspect, the application relates to a recombinant microorganism comprising at least one nucleic acid molecule encoding a polypeptide with keto-isovalerate decarboxylase (KIVD) activity, wherein said polypeptide is at least about 65% identical to SEQ ID NO: 101. In one embodiment, the polypeptide with keto-isovalerate decarboxylase (KIVD) activity is derived from an Enterobacteriaceae. In a specific embodiment, the polypeptide with keto-isovalerate decarboxylase (KIVD) activity is derived from Enterobacteriaceae bacterium 9_{—}2_{—}54FAA.

In yet another aspect, the application relates to a recombinant microorganism comprising at least one nucleic acid molecule encoding a polypeptide with keto-isovalerate decarboxylase (KIVD) activity, wherein said polypeptide is at least about 65% identical to a polypeptide selected from SEQ ID NOs: 102-143. In one embodiment, the polypeptide with keto-isovalerate decarboxylase (KIVD) activity is derived from a genus selected from Salmonella, Klebsiella, Enterobacter, Cronobacter, or Citrobacter. In a specific embodiment, the polypeptide with keto-isovalerate decarboxylase (KIVD) activity is derived from Salmonella enterica, Klebsiella pneumoniae, Klebsiella veriicola, Klebsiella sp. 1_{—}1_{—}55, Klebsiella sp. MS 92-3, Enterobacter aerogenes, Enterobacter cancerogenus, Enterobacter sp. 638, Enterobacter cloacae, Enterobacter hormaechei, Cronobacter turicensis, or Cronobacter sakazakii.

In yet another aspect, the application relates to a recombinant microorganism comprising at least one nucleic acid molecule encoding a polypeptide with keto-isovalerate decarboxylase (KIVD) activity, wherein said polypeptide is at least about 65% identical to a polypeptide selected from SEQ ID NOs: 144-149. In one embodiment, the polypeptide with keto-isovalerate decarboxylase (KIVD) activity is derived from the genus Pantoea. In a specific embodiment, the polypeptide with keto-isovalerate decarboxylase (KIVD) activity is derived from Pantoea sp. aB, Pantoea ananatis, Pantoea sp. At-9b, Pantoea agglomerans, or Pantoea vagans.

In yet another aspect, the application relates to a recombinant microorganism comprising at least one nucleic acid molecule encoding a polypeptide with keto-isovalerate decarboxylase (KIVD) activity, wherein said polypeptide is at least about 65% identical to a polypeptide selected from SEQ ID NOs: 150-155. In one embodiment, the polypeptide with keto-isovalerate decarboxylase (KIVD) activity is derived from the genus Erwinia. In a specific embodiment, the polypeptide with keto-isovalerate decarboxylase (KIVD) activity is derived from Erwinia amylovora, Erwinia tasmaniensis, Erwinia sp. Ejp617, Erwinia billingiae, or Erwinia pyrifoliae.

In yet another aspect, the application relates to a recombinant microorganism comprising at least one nucleic acid molecule encoding a polypeptide with keto-isovalerate decarboxylase (KIVD) activity, wherein said polypeptide is at least about 65% identical to a polypeptide selected from SEQ ID NOs: 156-158. In one embodiment, the polypeptide with keto-isovalerate decarboxylase (KIVD) activity is derived from the genus Pectobacterium. In a specific embodiment, the polypeptide with keto-isovalerate decarboxylase (KIVD) activity is derived from Pectobacterium carotovorum or Pectobacterium atrosepticum.

In yet another aspect, the application relates to a recombinant microorganism comprising at least one nucleic acid molecule encoding a polypeptide with keto-isovalerate decarboxylase (KIVD) activity, wherein said polypeptide is at least about 65% identical to SEQ ID NO: 159. In one embodiment, the polypeptide with keto-isovalerate decarboxylase (KIVD) activity is derived from the genus Rahnella. In a specific embodiment, the polypeptide with keto-isovalerate decarboxylase (KIVD) activity is derived from Rahnella sp. Y9602.

In yet another aspect, the application relates to a recombinant microorganism comprising at least one nucleic acid molecule encoding a polypeptide with keto-isovalerate decarboxylase (KIVD) activity, wherein said polypeptide is at least about 65% identical to a polypeptide selected from SEQ ID NOs: 160-172. In one embodiment, the polypeptide with keto-isovalerate decarboxylase (KIVD) activity is derived from a genus selected from Yersinia, Serratia, or Nasonia. In a specific embodiment, the polypeptide with keto-isovalerate decarboxylase (KIVD) activity is derived from Yersinia aldovae, Yersinia rohdei, Yersinia enterocolitica, Yersinia kristensenii, Yersinia mollaretii, Serratia symbiotica, Serratia sp. AS12, Serratia odorifera, Serratia proteamaculans, or Nasonia vitripennis.

In yet another aspect, the application relates to a recombinant microorganism comprising at least one nucleic acid molecule encoding a polypeptide with keto-isovalerate decarboxylase (KIVD) activity, wherein said polypeptide is at least about 65% identical to SEQ ID NO: 173. In one embodiment, the polypeptide with keto-isovalerate decarboxylase (KIVD) activity is derived from the genus Kineococcus. In a specific embodiment, the polypeptide with keto-isovalerate decarboxylase (KIVD) activity is derived from Kineococcus radiotolerans.

In yet another aspect, the application relates to a recombinant microorganism comprising at least one nucleic acid molecule encoding a polypeptide with keto-isovalerate decarboxylase (KIVD) activity, wherein said polypeptide is at least about 65% identical to a polypeptide selected from SEQ ID NOs: 174-177. In one embodiment, the polypeptide with keto-isovalerate decarboxylase (KIVD) activity is derived from the genus Psychrobacter. In a specific embodiment, the polypeptide with keto-isovalerate decarboxylase (KIVD) activity is derived from Psychrobacter arcticus, Psychrobacter cryohalolentis, Psychrobacter sp. PRwf-1, or Psychrobacter sp. 1501.

In yet another aspect, the application relates to a recombinant microorganism comprising at least one nucleic acid molecule encoding a polypeptide with keto-isovalerate decarboxylase (KIVD) activity, wherein said polypeptide is at least about 65% identical to SEQ ID NO: 178. In one embodiment, the polypeptide with keto-isovalerate decarboxylase (KIVD) activity is derived from the genus Corynebacterium. In a specific embodiment, the polypeptide with keto-isovalerate decarboxylase (KIVD) activity is derived from Corynebacterium striatum.

In yet another aspect, the application relates to a recombinant microorganism comprising at least one nucleic acid molecule encoding a polypeptide with keto-isovalerate decarboxylase (KIVD) activity, wherein said polypeptide is at least about 65% identical to SEQ ID NO: 179. In one embodiment, the polypeptide with keto-isovalerate decarboxylase (KIVD) activity is derived from the genus Corynebacterium. In a specific embodiment, the polypeptide with keto-isovalerate decarboxylase (KIVD) activity is derived from Corynebacterium kroppenstedtii.

In yet another aspect, the application relates to a recombinant microorganism comprising at least one nucleic acid molecule encoding a polypeptide with keto-isovalerate decarboxylase (KIVD) activity, wherein said polypeptide is at least about 65% identical to SEQ ID NO: 180. In one embodiment, the polypeptide with keto-isovalerate decarboxylase (KIVD) activity is derived from the genus Mycobacterium. In a specific embodiment, the polypeptide with keto-isovalerate decarboxylase (KIVD) activity is derived from Mycobacterium testaceum.

In yet another aspect, the application relates to a recombinant microorganism comprising at least one nucleic acid molecule encoding a polypeptide with keto-isovalerate decarboxylase (KIVD) activity, wherein said polypeptide is at least about 65% identical to SEQ ID NO: 181. In one embodiment, the polypeptide with keto-isovalerate decarboxylase (KIVD) activity is derived from the genus Nakamurella. In a specific embodiment, the polypeptide with keto-isovalerate decarboxylase (KIVD) activity is derived from Nakamurella multipartite.

In yet another aspect, the application relates to a recombinant microorganism comprising at least one nucleic acid molecule encoding a polypeptide with keto-isovalerate decarboxylase (KIVD) activity, wherein said polypeptide is at least about 65% identical to a polypeptide selected from SEQ ID NOs: 182-183. In one embodiment, the polypeptide with keto-isovalerate decarboxylase (KIVD) activity is derived from the genus Segniliparus. In a specific embodiment, the polypeptide with keto-isovalerate decarboxylase (KIVD) activity is derived from Segniliparus rotundus or Sengiliparus rugosus.

In yet another aspect, the application relates to a recombinant microorganism comprising at least one nucleic acid molecule encoding a polypeptide with keto-isovalerate decarboxylase (KIVD) activity, wherein said polypeptide is at least about 65% identical to a polypeptide selected from SEQ ID NOs: 184-196. In one embodiment, the polypeptide with keto-isovalerate decarboxylase (KIVD) activity is derived from the genus Mycobacterium. In a specific embodiment, the polypeptide with keto-isovalerate decarboxylase (KIVD) activity is derived from Mycobacterium marinurn, Mycobacterium tuberculosis, Mycobacterium avium, Mycobacterium kansasii, Mycobacterium leprae, Mycobacterium parascrofulaceum, Mycobacterium smegmatis, Mycobacterium ulcerans, or Mycobacterium intracellulare.

In yet another aspect, the application relates to a recombinant microorganism comprising at least one nucleic acid molecule encoding a polypeptide with keto-isovalerate decarboxylase (KIVD) activity, wherein said polypeptide is at least about 65% identical to a polypeptide selected from SEQ ID NOs: 198-208. In one embodiment, the polypeptide with keto-isovalerate decarboxylase (KIVD) activity is derived from the genus Francisella. In a specific embodiment, the polypeptide with keto-isovalerate decarboxylase (KIVD) activity is derived from Francisella novicida, Francisella tularensis, or Francisella philomiragia.

In yet another aspect, the application relates to a recombinant microorganism comprising at least one nucleic acid molecule encoding a polypeptide with keto-isovalerate decarboxylase (KIVD) activity, wherein said polypeptide is at least about 65% identical to SEQ ID NO: 209. In one embodiment, the polypeptide with keto-isovalerate decarboxylase (KIVD) activity is derived from the genus Beijerinckia. In a specific embodiment, the polypeptide with keto-isovalerate decarboxylase (KIVD) activity is derived from Beijerinckia indica.

In yet another aspect, the application relates to a recombinant microorganism comprising at least one nucleic acid molecule encoding a polypeptide with keto-isovalerate decarboxylase (KIVD) activity, wherein said polypeptide is at least about 65% identical to a polypeptide selected from SEQ ID NOs: 210-211. In one embodiment, the polypeptide with keto-isovalerate decarboxylase (KIVD) activity is derived from the genus Desulfovibrio.

In yet another aspect, the application relates to a recombinant microorganism comprising at least one nucleic acid molecule encoding a polypeptide with keto-isovalerate decarboxylase (KIVD) activity, wherein said polypeptide is at least about 65% identical to a polypeptide selected from SEQ ID NOs: 212-213. In one embodiment, the polypeptide with keto-isovalerate decarboxylase (KIVD) activity is derived from the genus Edwardsiella. In a specific embodiment, the polypeptide with keto-isovalerate decarboxylase (KIVD) activity is derived from Edwardsiella tarda or Edwardsiella ictaluri.

In yet another aspect, the application relates to a recombinant microorganism comprising at least one nucleic acid molecule encoding a polypeptide with keto-isovalerate decarboxylase (KIVD) activity, wherein said polypeptide is at least about 65% identical to SEQ ID NO: 214. In one embodiment, the polypeptide with keto-isovalerate decarboxylase (KIVD) activity is derived from the genus Singuliasphaera. In a specific embodiment, the polypeptide with keto-isovalerate decarboxylase (KIVD) activity is derived from Singuliasphaera acidiphila.

In another aspect, the application relates to a decarboxylase enzyme which has been modified or mutated to increase the ability of the enzyme to preferentially utilize keto-isovalerate as its substrate. Examples of such enzymes include decarboxylase enzymes having one or more modifications or mutations at positions corresponding to amino acids selected from: (a) aspartic acid 26 of the L. lactis KIVD (SEQ ID NO: 197); (b) histidine 112 of the L. lactis KIVD (SEQ ID NO: 197); (c) histidine 113 of the L. lactis KIVD (SEQ ID NO: 197); (d) glycine 402 of the L. lactis KIVD (SEQ ID NO: 197); and (e) glutamic acid 462 of the L. lactis KIVD (SEQ ID NO: 197).

In yet another aspect, the application relates to a decarboxylase enzyme which has been modified or mutated to alter one or more substrate-specificity residues. Examples of such enzymes include decarboxylase enzymes having one or more modifications or mutations at positions corresponding to amino acids selected from: (a) serine 286 of the L. lactis KIVD (SEQ ID NO: 197); (b) glutamine 377 of the L. lactis KIVD (SEQ ID NO: 197); (c) phenylalanine 381 of the L. lactis KIVD (SEQ ID NO: 197); (d) valine 461 of the L. lactis KIVD (SEQ ID NO: 197); (e) isoleucine 465 of the L. lactis KIVD (SEQ ID NO: 197); (f) methionine 538 of the L. lactis KIVD (SEQ ID NO: 197); and (g) phenylalanine 542 of the L. lactis KIVD (SEQ ID NO: 197).

In one embodiment, the decarboxylase enzyme contains a modification or mutation at the amino acid corresponding to position 26 of the L. lactis KIVD (SEQ ID NO: 197). In another embodiment, the decarboxylase enzyme contains a modification or mutation at the amino acid corresponding to position 112 of the L. lactis KIVD (SEQ ID NO: 197). In yet another embodiment, the decarboxylase enzyme contains a modification or mutation at the amino acid corresponding to position 113 of the L. lactis KIVD (SEQ ID NO: 197). In yet another embodiment, the decarboxylase enzyme contains a modification or mutation at the amino acid corresponding to position 286 of the L. lactis KIVD (SEQ ID NO: 197). In yet another embodiment, the decarboxylase enzyme contains a modification or mutation at the amino acid corresponding to position 377 of the L. lactis KIVD (SEQ ID NO: 197). In yet another embodiment, the decarboxylase enzyme contains a modification or mutation at the amino acid corresponding to position 381 of the L. lactis KIVD (SEQ ID NO: 197). In yet another embodiment, the decarboxylase enzyme contains a modification or mutation at the amino acid corresponding to position 402 of the L. lactis KIVD (SEQ ID NO: 197). In yet another embodiment, the decarboxylase enzyme contains a modification or mutation at the amino acid corresponding to position 461 of the L. lactis KIVD (SEQ ID NO: 197). In yet another embodiment, the decarboxylase enzyme contains a modification or mutation at the amino acid corresponding to position 462 of the L. lactis KIVD (SEQ ID NO: 197). In yet another embodiment, the decarboxylase enzyme contains a modification or mutation at the amino acid corresponding to position 465 of the L. lactis KIVD (SEQ ID NO: 197). In yet another embodiment, the decarboxylase enzyme contains a modification or mutation at the amino acid corresponding to position 538 of the L. lactis KIVD (SEQ ID NO: 197). In yet another embodiment, the decarboxylase enzyme contains a modification or mutation at the amino acid corresponding to position 542 of the L. lactis KIVD (SEQ ID NO: 197).

In one embodiment, the decarboxylase enzyme contains two or more modifications or mutations at the amino acids corresponding to the positions described above. In another embodiment, the decarboxylase enzyme contains three or more modifications or mutations at the amino acids corresponding to the positions described above. In yet another embodiment, the decarboxylase enzyme contains four or more modifications or mutations at the amino acids corresponding to the positions described above. In yet another embodiment, the decarboxylase enzyme contains five or more modifications or mutations at the amino acids corresponding to the positions described above. In yet another embodiment, the decarboxylase enzyme contains six or more modifications or mutations at the amino acids corresponding to the positions described above. In yet another embodiment, the decarboxylase enzyme contains seven or more modifications or mutations at the amino acids corresponding to the positions described above. In yet another embodiment, the decarboxylase enzyme contains eight or more modifications or mutations at the amino acids corresponding to the positions described above. In yet another embodiment, the decarboxylase enzyme contains nine or more modifications or mutations at the amino acids corresponding to the positions described above. In yet another embodiment, the decarboxylase enzyme contains ten or more modifications or mutations at the amino acids corresponding to the positions described above. In yet another embodiment, the decarboxylase enzyme contains eleven or more modifications or mutations at the amino acids corresponding to the positions described above. In yet another embodiment, the decarboxylase enzyme contains twelve modifications or mutations at the amino acids corresponding to the positions described above.

In yet another aspect, the application relates to a decarboxylase enzyme which has been modified or mutated to alter one or more substrate-specificity residues. Examples of such enzymes include decarboxylase enzymes having one or more modifications or mutations at positions corresponding to amino acids selected from: (a) phenylalanine 305 of the F. novicida decarboxylase (SEQ ID NO: 198); (b) threonine 397 of the F. novicida decarboxylase (SEQ ID NO: 198); (c) serine 401 of the F. novicida decarboxylase (SEQ ID NO: 198); (d) isoleucine 481 of the F. novicida decarboxylase (SEQ ID NO: 198); (e) leucine 485 of the F. novicida decarboxylase (SEQ ID NO: 198); (f) phenylalanine 556 of the F. novicida decarboxylase (SEQ ID NO: 198); and (g) leucine 560 of the F. novicida decarboxylase (SEQ ID NO: 198).

In one embodiment, the decarboxylase enzyme contains a modification or mutation at the amino acid corresponding to position 305 of the F. novicida decarboxylase (SEQ ID NO: 198). In another embodiment, the decarboxylase enzyme contains a modification or mutation at the amino acid corresponding to position 397 of the F. novicida decarboxylase (SEQ ID NO: 198). In yet another embodiment, the decarboxylase enzyme contains a modification or mutation at the amino acid corresponding to position 401 of the F. novicida decarboxylase (SEQ ID NO: 198). In yet another embodiment, the decarboxylase enzyme contains a modification or mutation at the amino acid corresponding to position 481 of the F. novicida decarboxylase (SEQ ID NO: 198). In yet another embodiment, the decarboxylase enzyme contains a modification or mutation at the amino acid corresponding to position 481 of the F. novicida decarboxylase (SEQ ID NO: 198). In yet another embodiment, the decarboxylase enzyme contains a modification or mutation at the amino acid corresponding to position 485 of the F. novicida decarboxylase (SEQ ID NO: 198). In yet another embodiment, the decarboxylase enzyme contains a modification or mutation at the amino acid corresponding to position 556 of the F. novicida decarboxylase (SEQ ID NO: 198). In yet another embodiment, the decarboxylase enzyme contains a modification or mutation at the amino acid corresponding to position 560 of the F. novicida decarboxylase (SEQ ID NO: 198). In one embodiment, the decarboxylase enzyme contains two or more modifications or mutations at the amino acids corresponding to the positions described above. In another embodiment, the decarboxylase enzyme contains three or more modifications or mutations at the amino acids corresponding to the positions described above. In yet another embodiment, the decarboxylase enzyme contains four or more modifications or mutations at the amino acids corresponding to the positions described above. In yet another embodiment, the decarboxylase enzyme contains five or more modifications or mutations at the amino acids corresponding to the positions described above. In yet another embodiment, the decarboxylase enzyme contains six or more modifications or mutations at the amino acids corresponding to the positions described above. In yet another embodiment, the decarboxylase enzyme contains seven modifications or mutations at the amino acids corresponding to the positions described above.

In yet another aspect, the application relates to a pyruvate decarboxylase (PDC) enzyme which has been modified or mutated to alter one or more substrate-specificity residues. Examples of such enzymes include enzymes having one or more modifications or mutations at positions corresponding to amino acids selected from: (a) phenylalanine 292 of the S. cerevisiae PDC1 (SEQ ID NO: 241); (b) threonine 388 of the S. cerevisiae PDC1 (SEQ ID NO: 241); (c) alanine 392 of the S. cerevisiae PDC1 (SEQ ID NO: 241); (d) serine 408 of the S. cerevisiae PDC1 (SEQ ID NO: 241); (e) valine 410 of the S. cerevisiae PDC1 (SEQ ID NO: 241); (f) isoleucine 476 of the S. cerevisiae PDC1 (SEQ ID NO: 241); (g) glutamine 552 of the S. cerevisiae PDC1 (SEQ ID NO: 241); and (h) threonine 556 of the S. cerevisiae PDC1 (SEQ ID NO: 241). In one embodiment, the pyruvate decarboxylase enzyme to be modified is obtained from a yeast microorganism. In a further embodiment, the pyruvate decarboxylase enzyme to be modified is obtained from a yeast microorganism classified into a genera selected from the group consisting of Saccharomyces, Kluyveromyces, Candida, Pichia, Issatchenkia, Debaryomyces, Hansenula, Pachysolen, Yarrowia, Schizosaccharomyces, Tricosporon, Rhodotorula, and Myxozyma. In another further embodiment, the pyruvate decarboxylase enzyme to be modified is obtained from a Saccharomyces yeast. In an exemplary embodiment, the pyruvate decarboxylase to be modified is obtained from Saccharomyces cerevisiae. In another exemplary embodiment, the pyruvate decarboxylase to be modified is PDC1, PDC5, or PDC6 of S. cerevisiae.

In one embodiment, the pyruvate decarboxylase enzyme contains a modification or mutation at the amino acid corresponding to position 292 of the S. cerevisiae PDC1 (SEQ ID NO: 241). In another embodiment, the pyruvate decarboxylase enzyme contains a modification or mutation at the amino acid corresponding to position 388 of the S. cerevisiae PDC1 (SEQ ID NO: 241). In yet another embodiment, the pyruvate decarboxylase enzyme contains a modification or mutation at the amino acid corresponding to position 392 of the S. cerevisiae PDC1 (SEQ ID NO: 241). In yet another embodiment, the pyruvate decarboxylase enzyme contains a modification or mutation at the amino acid corresponding to position 408 of the S. cerevisiae PDC1 (SEQ ID NO: 241). In yet another embodiment, the pyruvate decarboxylase enzyme contains a modification or mutation at the amino acid corresponding to position 410 of the S. cerevisiae PDC1 (SEQ ID NO: 241). In yet another embodiment, the pyruvate decarboxylase enzyme contains a modification or mutation at the amino acid corresponding to position 476 of the S. cerevisiae PDC1 (SEQ ID NO: 241). In yet another embodiment, the pyruvate decarboxylase enzyme contains a modification or mutation at the amino acid corresponding to position 552 of the S. cerevisiae PDC1 (SEQ ID NO: 241). In yet another embodiment, the pyruvate decarboxylase enzyme contains a modification or mutation at the amino acid corresponding to position 556 of the S. cerevisiae PDC1 (SEQ ID NO: 241). In one embodiment, the pyruvate decarboxylase enzyme contains two or more modifications or mutations at the amino acids corresponding to the positions described above. In another embodiment, the pyruvate decarboxylase enzyme contains three or more modifications or mutations at the amino acids corresponding to the positions described above. In yet another embodiment, the pyruvate decarboxylase enzyme contains four or more modifications or mutations at the amino acids corresponding to the positions described above. In yet another embodiment, the pyruvate decarboxylase enzyme contains five or more modifications or mutations at the amino acids corresponding to the positions described above. In yet another embodiment, the pyruvate decarboxylase enzyme contains six or more modifications or mutations at the amino acids corresponding to the positions described above. In yet another embodiment, the pyruvate decarboxylase enzyme contains seven or more modifications or mutations at the amino acids corresponding to the positions described above. In yet another embodiment, the pyruvate decarboxylase enzyme contains eight modifications or mutations at the amino acids corresponding to the positions described above.

In another aspect, the application relates to a recombinant microorganism comprising at least one nucleic acid molecule encoding a decarboxylase enzyme having one or more modifications or mutations at positions corresponding to amino acids selected from: (a) aspartic acid 26 of the L. lactis KIVD (SEQ ID NO: 197); (b) histidine 112 of the L. lactis KIVD (SEQ ID NO: 197); (c) histidine 113 of the L. lactis KIVD (SEQ ID NO: 197); (d) glycine 402 of the L. lactis KIVD (SEQ ID NO: 197); and (e) glutamic acid 462 of the L. lactis KIVD (SEQ ID NO: 197).

In yet another aspect, the application relates to a recombinant microorganism comprising at least one nucleic acid molecule encoding a decarboxylase enzyme having one or more modifications or mutations at positions corresponding to amino acids selected from: (a) serine 286 of the L. lactis KIVD (SEQ ID NO: 197); (b) glutamine 377 of the L. lactis KIVD (SEQ ID NO: 197); (c) phenylalanine 381 of the L. lactis KIVD (SEQ ID NO: 197); (d) valine 461 of the L. lactis KIVD (SEQ ID NO: 197); (e) isoleucine 465 of the L. lactis KIVD (SEQ ID NO: 197); (f) methionine 538 of the L. lactis KIVD (SEQ ID NO: 197); and (g) phenylalanine 542 of the L. lactis KIVD (SEQ ID NO: 197).

In yet another aspect, the application relates to a recombinant microorganism comprising at least one nucleic acid molecule encoding a decarboxylase enzyme having one or more modifications or mutations at positions corresponding to amino acids selected from: (a) phenylalanine 305 of the F. novicida decarboxylase (SEQ ID NO: 198); (b) threonine 397 of the F. novicida decarboxylase (SEQ ID NO: 198); (c) serine 401 of the F. novicida decarboxylase (SEQ ID NO: 198); (d) isoleucine 481 of the F. novicida decarboxylase (SEQ ID NO: 198); (e) leucine 485 of the F. novicida decarboxylase (SEQ ID NO: 198); (f) phenylalanine 556 of the F. novicida decarboxylase (SEQ ID NO: 198); and (g) leucine 560 of the F. novicida decarboxylase (SEQ ID NO: 198).

In yet another aspect, the application relates to a recombinant microorganism comprising at least one nucleic acid molecule encoding a decarboxylase enzyme having one or more modifications or mutations at positions corresponding to amino acids selected from: (a) phenylalanine 292 of the S. cerevisiae PDC1 (SEQ ID NO: 241); (b) threonine 388 of the S. cerevisiae PDC1 (SEQ ID NO: 241); (c) alanine 392 of the S. cerevisiae PDC1 (SEQ ID NO: 241); (d) serine 408 of the S. cerevisiae PDC1 (SEQ ID NO: 241); (e) valine 410 of the S. cerevisiae PDC1 (SEQ ID NO: 241); (f) isoleucine 476 of the S. cerevisiae PDC1 (SEQ ID NO: 241); (g) glutamine 552 of the S. cerevisiae PDC1 (SEQ ID NO: 241); and (h) threonine 556 of the S. cerevisiae PDC1 (SEQ ID NO: 241).

In various embodiments described herein, the recombinant microorganism comprises an isobutanol producing metabolic pathway. In one embodiment, the isobutanol producing metabolic pathway comprises at least one exogenous gene encoding a polypeptide that catalyzes a step in the conversion of pyruvate to isobutanol. In another embodiment, the isobutanol producing metabolic pathway comprises at least two exogenous genes encoding polypeptides that catalyze steps in the conversion of pyruvate to isobutanol. In yet another embodiment, the isobutanol producing metabolic pathway comprises at least three exogenous genes encoding polypeptides that catalyze steps in the conversion of pyruvate to isobutanol. In yet another embodiment, the isobutanol producing metabolic pathway comprises at least four exogenous genes encoding polypeptides that catalyze steps in the conversion of pyruvate to isobutanol. In yet another embodiment, the isobutanol producing metabolic pathway comprises at least five exogenous genes encoding polypeptides that catalyze steps in the conversion of pyruvate to isobutanol. In yet another embodiment, all of the isobutanol producing metabolic pathway steps in the conversion of pyruvate to isobutanol are converted by exogenously encoded enzymes. In an exemplary embodiment, at least one of the exogenously encoded enzymes is a polypeptide with keto-isovalerate decarboxylase (KIVD) activity, wherein said polypeptide is at least about 65% identical to a polypeptide selected from SEQ ID NOs 1-214. In another exemplary embodiment, at least one of the exogenously encoded enzymes is a decarboxylase enzyme having one or more modifications or mutations at positions corresponding to amino acids selected from: (a) aspartic acid 26 of the L. lactis KIVD (SEQ ID NO: 197); (b) histidine 112 of the L. lactis KIVD (SEQ ID NO: 197); (c) histidine 113 of the L. lactis KIVD (SEQ ID NO: 197); (d) glycine 402 of the L. lactis KIVD (SEQ ID NO: 197); and (e) glutamic acid 462 of the L. lactis KIVD (SEQ ID NO: 197). In yet another exemplary embodiment, at least one of the exogenously encoded enzymes is a decarboxylase enzyme having one or more modifications or mutations at positions corresponding to amino acids selected from: (a) serine 286 of the L. lactis KIVD (SEQ ID NO: 197); (b) glutamine 377 of the L. lactis KIVD (SEQ ID NO: 197); (c) phenylalanine 381 of the L. lactis KIVD (SEQ ID NO: 197); (d) valine 461 of the L. lactis KIVD (SEQ ID NO: 197); (e) isoleucine 465 of the L. lactis KIVD (SEQ ID NO: 197); (f) methionine 538 of the L. lactis KIVD (SEQ ID NO: 197); and (g) phenylalanine 542 of the L. lactis KIVD (SEQ ID NO: 197). In yet another exemplary embodiment, at least one of the exogenously encoded enzymes is a decarboxylase enzyme having one or more modifications or mutations at positions corresponding to amino acids selected from: (a) phenylalanine 305 of the F. novicida decarboxylase (SEQ ID NO: 198); (b) threonine 397 of the F. novicida decarboxylase (SEQ ID NO: 198); (c) serine 401 of the F. novicida decarboxylase (SEQ ID NO: 198); (d) isoleucine 481 of the F. novicida decarboxylase (SEQ ID NO: 198); (e) leucine 485 of the F. novicida decarboxylase (SEQ ID NO: 198); (f) phenylalanine 556 of the F. novicida decarboxylase (SEQ ID NO: 198); and (g) leucine 560 of the F. novicida decarboxylase (SEQ ID NO: 198). In yet another exemplary embodiment, at least one of the exogenously encoded enzymes is a decarboxylase enzyme having one or more modifications or mutations at positions corresponding to amino acids selected from: (a) phenylalanine 292 of the S. cerevisiae PDC1 (SEQ ID NO: 241); (b) threonine 388 of the S. cerevisiae PDC1 (SEQ ID NO: 241); (c) alanine 392 of the S. cerevisiae PDC1 (SEQ ID NO: 241); (d) serine 408 of the S. cerevisiae PDC1 (SEQ ID NO: 241); (e) valine 410 of the S. cerevisiae PDC1 (SEQ ID NO: 241); (f) isoleucine 476 of the S. cerevisiae PDC1 (SEQ ID NO: 241); (g) glutamine 552 of the S. cerevisiae PDC1 (SEQ ID NO: 241); and (h) threonine 556 of the S. cerevisiae PDC1 (SEQ ID NO: 241).

In one embodiment, one or more of the isobutanol pathway genes encodes an enzyme that is localized to the cytosol. In one embodiment, the recombinant microorganisms comprise an isobutanol producing metabolic pathway with at least one isobutanol pathway enzyme localized in the cytosol. In another embodiment, the recombinant microorganisms comprise an isobutanol producing metabolic pathway with at least two isobutanol pathway enzymes localized in the cytosol. In yet another embodiment, the recombinant microorganisms comprise an isobutanol producing metabolic pathway with at least three isobutanol pathway enzymes localized in the cytosol. In yet another embodiment, the recombinant microorganisms comprise an isobutanol producing metabolic pathway with at least four isobutanol pathway enzymes localized in the cytosol. In an exemplary embodiment, the recombinant microorganisms comprise an isobutanol producing metabolic pathway with five isobutanol pathway enzymes localized in the cytosol. In yet another exemplary embodiment, the recombinant microorganisms comprise an isobutanol producing metabolic pathway with all isobutanol pathway enzymes localized in the cytosol.

In various embodiments described herein, the isobutanol pathway genes may encode enzyme(s) selected from the group consisting of acetolactate synthase (ALS), ketol-acid reductoisomerase (KARI), dihydroxyacid dehydratase (DHAD), 2-keto-acid decarboxylase, e.g., keto-isovalerate decarboxylase (KIVD), and alcohol dehydrogenase (ADH). In one embodiment, the KARI is an NADH-dependent KARI (NKR). In another embodiment, the ADH is an NADH-dependent ADH. In yet another embodiment, the KARI is an NADH-dependent KARI (NKR) and the ADH is an NADH-dependent ADH. In an exemplary embodiment, the 2-keto-acid decarboxylase is a polypeptide with keto-isovalerate decarboxylase (KIVD) activity, wherein said polypeptide is at least about 65% identical to a polypeptide selected from SEQ ID NOs 1-214. In another exemplary embodiment, the 2-keto-acid decarboxylase a decarboxylase enzyme having one or more modifications or mutations at positions corresponding to amino acids selected from: (a) aspartic acid 26 of the L. lactis KIVD (SEQ ID NO: 197); (b) histidine 112 of the L. lactis KIVD (SEQ ID NO: 197); (c) histidine 113 of the L. lactis KIVD (SEQ ID NO: 197); (d) glycine 402 of the L. lactis KIVD (SEQ ID NO: 197); and (e) glutamic acid 462 of the L. lactis KIVD (SEQ ID NO: 197). In yet another exemplary embodiment, the 2-keto-acid decarboxylase is a decarboxylase enzyme having one or more modifications or mutations at positions corresponding to amino acids selected from: (a) serine 286 of the L. lactis KIVD (SEQ ID NO: 197); (b) glutamine 377 of the L. lactis KIVD (SEQ ID NO: 197); (c) phenylalanine 381 of the L. lactis KIVD (SEQ ID NO: 197); (d) valine 461 of the L. lactis KIVD (SEQ ID NO: 197); (e) isoleucine 465 of the L. lactis KIVD (SEQ ID NO: 197); (f) methionine 538 of the L. lactis KIVD (SEQ ID NO: 197); and (g) phenylalanine 542 of the L. lactis KIVD (SEQ ID NO: 197). In yet another exemplary embodiment, the 2-keto-acid decarboxylase is a decarboxylase enzyme having one or more modifications or mutations at positions corresponding to amino acids selected from: (a) phenylalanine 305 of the F. novicida decarboxylase (SEQ ID NO: 198); (b) threonine 397 of the F. novicida decarboxylase (SEQ ID NO: 198); (c) serine 401 of the F. novicida decarboxylase (SEQ ID NO: 198); (d) isoleucine 481 of the F. novicida decarboxylase (SEQ ID NO: 198); (e) leucine 485 of the F. novicida decarboxylase (SEQ ID NO: 198); (f) phenylalanine 556 of the F. novicida decarboxylase (SEQ ID NO: 198); and (g) leucine 560 of the F. novicida decarboxylase (SEQ ID NO: 198). In yet another exemplary embodiment, the 2-keto-acid decarboxylase is a decarboxylase enzyme having one or more modifications or mutations at positions corresponding to amino acids selected from: (a) phenylalanine 292 of the S. cerevisiae PDC1 (SEQ ID NO: 241); (b) threonine 388 of the S. cerevisiae PDC1 (SEQ ID NO: 241); (c) alanine 392 of the S. cerevisiae PDC1 (SEQ ID NO: 241); (d) serine 408 of the S. cerevisiae PDC1 (SEQ ID NO: 241); (e) valine 410 of the S. cerevisiae PDC1 (SEQ ID NO: 241); (f) isoleucine 476 of the S. cerevisiae PDC1 (SEQ ID NO: 241); (g) glutamine 552 of the S. cerevisiae PDC1 (SEQ ID NO: 241); and (h) threonine 556 of the S. cerevisiae PDC1 (SEQ ID NO: 241).

In various embodiments described herein, the recombinant microorganisms of the invention that comprise an isobutanol producing metabolic pathway may be further engineered to reduce or eliminate the expression or activity of one or more enzymes selected from a pyruvate decarboxylase (PDC), a glycerol-3-phosphate dehydrogenase (GPD), a 3-keto acid reductase (3-KAR), or an aldehyde dehydrogenase (ALDH).

In one embodiment, the recombinant microorganisms may be recombinant prokaryotic microorganisms. In another embodiment, the recombinant microorganisms may be recombinant eukaryotic microorganisms. In a further embodiment, the recombinant eukaryotic microorganisms may be recombinant yeast microorganisms.

In some embodiments, the recombinant yeast microorganisms may be members of the Saccharomyces clade, Saccharomyces sensu stricto microorganisms, Crabtree-negative yeast microorganisms, Crabtree-positive yeast microorganisms, post-WGD (whole genome duplication) yeast microorganisms, pre-WGD (whole genome duplication) yeast microorganisms, and non-fermenting yeast microorganisms.

In some embodiments, the recombinant microorganisms may be yeast recombinant microorganisms of the Saccharomyces clade.

In some embodiments, the recombinant microorganisms may be Saccharomyces sensu stricto microorganisms. In one embodiment, the Saccharomyces sensu stricto is selected from the group consisting of S. cerevisiae, S. kudriavzevii, S. mikatae, S. bayanus, S. uvarum, S. carocanis and hybrids thereof.

In some embodiments, the recombinant microorganisms may be Crabtree-negative recombinant yeast microorganisms. In one embodiment, the Crabtree-negative yeast microorganism is classified into a genera selected from the group consisting of Saccharomyces, Kluyveromyces, Pichia, Issatchenkia, Hansenula, or Candida. In additional embodiments, the Crabtree-negative yeast microorganism is selected from Saccharomyces kluyveri, Kluyveromyces lactis, Kluyveromyces marxianus, Pichia anomala, Pichia stipitis, Hansenula anomala, Candida utilis and Kluyveromyces waltii.

In some embodiments, the recombinant microorganisms may be Crabtree-positive recombinant yeast microorganisms. In one embodiment, the Crabtree-positive yeast microorganism is classified into a genera selected from the group consisting of Saccharomyces, Kluyveromyces, Zygosaccharomyces, Debaryomyces, Candida, Pichia and Schizosaccharomyces. In additional embodiments, the Crabtree-positive yeast microorganism is selected from the group consisting of Saccharomyces cerevisiae, Saccharomyces uvarum, Saccharomyces bayanus, Saccharomyces paradoxus, Saccharomyces castelli, Kluyveromyces thermotolerans, Candida glabrata, Z. bailli, Z. rouxii, Debaryomyces hansenii, Pichia pastorius, Schizosaccharomyces pombe, and Saccharomyces uvarum.

In some embodiments, the recombinant microorganisms may be post-WGD (whole genome duplication) yeast recombinant microorganisms. In one embodiment, the post-WGD yeast recombinant microorganism is classified into a genera selected from the group consisting of Saccharomyces or Candida. In additional embodiments, the post-WGD yeast is selected from the group consisting of Saccharomyces cerevisiae, Saccharomyces uvarum, Saccharomyces bayanus, Saccharomyces paradoxus, Saccharomyces castelli, and Candida glabrata.

In some embodiments, the recombinant microorganisms may be pre-WGD (whole genome duplication) yeast recombinant microorganisms. In one embodiment, the pre-WGD yeast recombinant microorganism is classified into a genera selected from the group consisting of Saccharomyces, Kluyveromyces, Candida, Pichia, Issatchenkia, Debaryomyces, Hansenula, Pachysolen, Yarrowia and Schizosaccharomyces. In additional embodiments, the pre-WGD yeast is selected from the group consisting of Saccharomyces kluyveri, Kluyveromyces thermotolerans, Kluyveromyces marxianus, Kluyveromyces waltii, Kluyveromyces lactis, Candida tropicalis, Pichia pastoris, Pichia anomala, Pichia stipitis, Issatchenkia orientalis, Issatchenkia occidentalis, Debaryomyces hansenii, Hansenula anomala, Pachysolen tannophilis, Yarrowia lipolytica, and Schizosaccharomyces pombe.

In some embodiments, the recombinant microorganisms may be microorganisms that are non-fermenting yeast microorganisms, including, but not limited to those, classified into a genera selected from the group consisting of Tricosporon, Rhodotorula, Myxozyma, or Candida. In a specific embodiment, the non-fermenting yeast is C. xestobii.

In another aspect, the present invention provides methods of producing isobutanol using a recombinant microorganism as described herein. In one embodiment, the method includes cultivating the recombinant microorganism in a culture medium containing a feedstock providing the carbon source until a recoverable quantity of isobutanol is produced and optionally, recovering the isobutanol. In one embodiment, the microorganism produces isobutanol from a carbon source at a yield of at least about 5 percent theoretical. In another embodiment, the microorganism produces isobutanol at a yield of at least about 10 percent, at least about 15 percent, about least about 20 percent, at least about 25 percent, at least about 30 percent, at least about 35 percent, at least about 40 percent, at least about 45 percent, at least about 50 percent, at least about 55 percent, at least about 60 percent, at least about 65 percent, at least about 70 percent, at least about 75 percent, at least about 80 percent, at least about 85 percent, at least about 90 percent, at least about 95 percent, or at least about 97.5 percent theoretical.

In one embodiment, the recombinant microorganism converts the carbon source to isobutanol under aerobic conditions. In another embodiment, the recombinant microorganism converts the carbon source to isobutanol under microaerobic conditions. In yet another embodiment, the recombinant microorganism converts the carbon source to isobutanol under anaerobic conditions.

### BRIEF DESCRIPTION OF DRAWINGS

Illustrative embodiments of the invention are illustrated in the drawings, in which:

FIG. 1 illustrates an exemplary embodiment of an isobutanol pathway.

FIG. 2 illustrates an exemplary embodiment of an NADH-dependent isobutanol pathway.

FIG. 3 illustrates a phylogenetic tree of characterized proteins from Table 2. Boxes distinctly outline IPDC proteins, PDC proteins, and KIVD proteins. “In-group” defines an evolutionary clade and “out-group” defines an evolutionary grade used in subsequent analysis.

FIG. 4 illustrates the phylogenetic tree of the KIVD clade. Each tree node/leaf represents a distinct “hit group.” The SEQ designations in this figure do not correspond to the specific SEQ ID NO: designations provided herein.

FIG. 5 illustrates the active site of KdcA from L. lactis. This active site includes catalytic residues (green, i.e., D26, E49, H112, H113, and E462), the thiamin diphosphate cofactor (dark blue, i.e., TPP), and residues shaping substrate specificity (orange, i.e., S286, Q377, F381, V461, I465, M538, F542). Also included is pyruvate (cyan, i.e., immediately above the I465 residue) as found in the S. cerevisiae PDC model 2vk1. The residues closest to the variable portion of the substrate (i.e., the pyruvate methyl portion of the aliphatic portion of keto-isovalerate) are V461, Q377, I465, and F542. Despite the greater distance of the other residues, S286, F381, and M538, these also appear to impact specificity. For example, aromatic residues at these positions appear to contribute to the relatively strict preference for pyruvate of Zm_PDC.

FIG. 6 illustrates an overlay of the S. cerevisiae PDC with KdcA. Pyruvate is bound very near to the thiamin diphosphate. Catalytic side chains are shown in white. Residues at specificity locations are illustrated in green (Sc_PDC, i.e., F292, T388, and I476) or orange (KdcA, i.e., S292, Q388, and V476). Several mutations are very close to the substrate and play a role in allowing bulky beta-branched substrates: I476V, T388Q, and F292S. The other mutations are farther from the substrate. The farther mutations play a role in determining activity toward larger substrates (e.g., indolepyruvate). The farther sites also differ between different PDCs. Unlike Sc_PDC, Zm_PDC has large aromatic residues at these locations and has a reduced substrate spectrum with respect to Sc_PDC.

FIG. 7 illustrates the crystal structure of the Sc_PDC variant D28A in complex with the substrate pyruvate (blue). The thiamine diphosphate (yellow) and catalytic residues (green) are poised for catalysis. The spacefilling model demonstrates a tight fit around pyruvate.

FIG. 8 illustrates a sorted listing of polypeptides (SEQ ID NOS.: 271-778) likely to exhibit specific keto-isovalerate decarboxylase (KivD) activity.

FIG. 9 illustrates an alignment of the specificity amino acids from the L. lactis KivD (SEQ ID NOS.: 271-292). The specificity amino acids refer to the identity of the residue corresponding to S286, Q377, F381, V461, I465, M538, and F542 from the L. lactis KivD.

FIG. 10 illustrates the specific activity on KIV for a cross-section of decarboxylases as determined by in vitro testing.

FIG. 11 illustrates the specific activity on pyruvate for a cross-section of decarboxylases as determined by in vitro testing.

FIG. 12 illustrates the ratio of specific activity for KIV/pyruvate for a cross-section of decarboxylases as determined by in vitro testing.

FIG. 13 illustrates how partial model for the Francisella cf. novicida 3523 decarboxylase, created by modeling mutations (white sticks) onto the structure of LI_KdcA (2vbf). To approximate the KIV position, a KIV molecule was modeled using SHARPEN/OpenBabel to create the coordinates and PyMOL to adjust the torsions. The substrate was placed in accord with the observed ligand positions in 2vk1 and 2vbg.

FIG. 14 illustrates the python script used to calculate sequence entropy within decarboxylases described herein.

FIGS. 15-17 illustrate python scripts used to generate models for wild-type S. cerevisiae PDC1 given crystal structures for point mutations thereof.

FIG. 18 illustrates a python script used to model point mutations within the S. cerevisiae PDC1. The script illustrates the A392F mutation analysis, which is representative of the analysis conducted for other disclosed point mutations. The models allowed for mutated sidechains to select new conformations from an expanded Dunbrack rotamer library.

FIG. 19 illustrates a python script for protein design calculation of the S. cerevisiae PDC1. This protein design calculation identified the sequence and rotamer sidechain positions which minimize the energy according to the all-atom Rosetta energy model.

FIG. 20 illustrates a script specifying the protein design palette for the S. cerevisiae PDC1.

### DETAILED DESCRIPTION

As used herein and in the appended claims, the singular forms “a,” “an,” and “the” include plural referents unless the context clearly dictates otherwise. Thus, for example, reference to “a polynucleotide” includes a plurality of such polynucleotides and reference to “the microorganism” includes reference to one or more microorganisms, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

Any publications discussed above and throughout the text are provided solely for their disclosure prior to the filing date of the present application. Nothing herein is to be construed as an admission that the inventors are not entitled to antedate such disclosure by virtue of prior disclosure.

The term “microorganism” includes prokaryotic and eukaryotic microbial species from the Domains Archaea, Bacteria and Eucarya, the latter including yeast and filamentous fungi, protozoa, algae, or higher Protista. The terms “microbial cells” and “microbes” are used interchangeably with the term microorganism.

The term “prokaryotes” is art recognized and refers to cells which contain no nucleus or other cell organelles. The prokaryotes are generally classified in one of two domains, the Bacteria and the Archaea. The definitive difference between organisms of the Archaea and Bacteria domains is based on fundamental differences in the nucleotide base sequence in the 16S ribosomal RNA.

The term “Archaea” refers to a categorization of organisms of the division Mendosicutes, typically found in unusual environments and distinguished from the rest of the prokaryotes by several criteria, including the number of ribosomal proteins and the lack of muramic acid in cell walls. On the basis of ssrRNA analysis, the Archaea consist of two phylogenetically-distinct groups: Crenarchaeota and Euryarchaeota. On the basis of their physiology, the Archaea can be organized into three types: methanogens (prokaryotes that produce methane); extreme halophiles (prokaryotes that live at very high concentrations of salt (NaCl); and extreme (hyper) thermophiles (prokaryotes that live at very high temperatures). Besides the unifying archaeal features that distinguish them from Bacteria (i.e., no murein in cell wall, ester-linked membrane lipids, etc.), these prokaryotes exhibit unique structural or biochemical attributes which adapt them to their particular habitats. The Crenarchaeota consist mainly of hyperthermophilic sulfur-dependent prokaryotes and the Euryarchaeota contain the methanogens and extreme halophiles.

“Bacteria”, or “eubacteria”, refers to a domain of prokaryotic organisms. Bacteria include at least eleven distinct groups as follows: (1) Gram-positive (gram+) bacteria, of which there are two major subdivisions: (1) high G+C group (Actinomycetes, Mycobacteria, Micrococcus, others) (2) low G+C group (Bacillus, Clostridia, Lactobacillus, Staphylococci, Streptococci, Mycoplasmas); (2) Proteobacteria, e.g., Purple photosynthetic+non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; (11) Thermotoga and Thermosipho thermophiles.

“Gram-negative bacteria” include cocci, nonenteric rods, and enteric rods. The genera of Gram-negative bacteria include, for example, Neisseria, Spirillum, Pasteurella, Brucella, Yersinia, Francisella, Haemophilus, Bordetella, Escherichia, Salmonella, Shigella, Klebsiella, Proteus, Vibrio, Pseudomonas, Bacteroides, Acetobacter, Aerobacter, Agrobacterium, Azotobacter, Spirilla, Serratia, Vibrio, Rhizobium, Chlamydia, Rickettsia, Treponema, and Fusobacterium.

“Gram positive bacteria” include cocci, nonsporulating rods, and sporulating rods. The genera of gram positive bacteria include, for example, Actinomyces, Bacillus, Clostridium, Corynebacterium, Erysipelothrix, Lactobacillus, Listeria, Mycobacterium, Myxococcus, Nocardia, Staphylococcus, Streptococcus, and Streptomyces.

The term “genus” is defined as a taxonomic group of related species according to the Taxonomic Outline of Bacteria and Archaea (Garrity, G. M., Lilburn, T. G., Cole, J. R., Harrison, S. H., Euzeby, J., and Tindall, B. J. (2007) The Taxonomic Outline of Bacteria and Archaea. TOBA Release 7.7, March 2007. Michigan State University Board of Trustees. [http://www.taxonomicoutline.org/]).

The term “species” is defined as a collection of closely related organisms with greater than 97% 16S ribosomal RNA sequence homology and greater than 70% genomic hybridization and sufficiently different from all other organisms so as to be recognized as a distinct unit.

The terms “recombinant microorganism,” “modified microorganism,” and “recombinant host cell” are used interchangeably herein and refer to microorganisms that have been genetically modified to express or to overexpress endogenous polynucleotides, to express heterologous polynucleotides, such as those included in a vector, in an integration construct, or which have an alteration in expression of an endogenous gene. By “alteration” it is meant that the expression of the gene, or level of a RNA molecule or equivalent RNA molecules encoding one or more polypeptides or polypeptide subunits, or activity of one or more polypeptides or polypeptide subunits is up regulated or down regulated, such that expression, level, or activity is greater than or less than that observed in the absence of the alteration. For example, the term “alter” can mean “inhibit,” but the use of the word “alter” is not limited to this definition. It is understood that the terms “recombinant microorganism” and “recombinant host cell” refer not only to the particular recombinant microorganism but to the progeny or potential progeny of such a microorganism. Because certain modifications may occur in succeeding generations due to either mutation or environmental influences, such progeny may not, in fact, be identical to the parent cell, but are still included within the scope of the term as used herein.

The term “expression” with respect to a gene sequence refers to transcription of the gene and, as appropriate, translation of the resulting mRNA transcript to a protein. Thus, as will be clear from the context, expression of a protein results from transcription and translation of the open reading frame sequence. The level of expression of a desired product in a host cell may be determined on the basis of either the amount of corresponding mRNA that is present in the cell, or the amount of the desired product encoded by the selected sequence. For example, mRNA transcribed from a selected sequence can be quantitated by qRT-PCR or by Northern hybridization (see Sambrook et al., Molecular Cloning: A Laboratory Manual, Cold Spring Harbor Laboratory Press (1989)). Protein encoded by a selected sequence can be quantitated by various methods, e.g., by ELISA, by assaying for the biological activity of the protein, or by employing assays that are independent of such activity, such as western blotting or radioimmunoassay, using antibodies that recognize and bind the protein. See Sambrook et al., 1989, supra.

The term “overexpression” refers to an elevated level (e.g., aberrant level) of mRNAs encoding for a protein(s), and/or to elevated levels of protein(s) in cells as compared to similar corresponding unmodified cells expressing basal levels of mRNAs or having basal levels of proteins. In particular embodiments, mRNA(s) or protein(s) may be overexpressed by at least 2-fold, 3-fold, 4-fold, 5-fold, 6-fold, 8-fold, 10-fold, 12-fold, 15-fold or more in microorganisms engineered to exhibit increased gene mRNA, protein, and/or activity.

As used herein and as would be understood by one of ordinary skill in the art, “reduced activity and/or expression” of a protein such as an enzyme can mean either a reduced specific catalytic activity of the protein (e.g. reduced activity) and/or decreased concentrations of the protein in the cell (e.g. reduced expression). As would be understood by one or ordinary skill in the art, the reduced activity of a protein in a cell may result from decreased concentrations of the protein in the cell.

The term “wild-type microorganism” describes a cell that occurs in nature, i.e. a cell that has not been genetically modified. A wild-type microorganism can be genetically modified to express or overexpress a first target enzyme. This microorganism can act as a parental microorganism in the generation of a microorganism modified to express or overexpress a second target enzyme. In turn, the microorganism modified to express or overexpress a first and a second target enzyme can be modified to express or overexpress a third target enzyme.

Accordingly, a “parental microorganism” functions as a reference cell for successive genetic modification events. Each modification event can be accomplished by introducing a nucleic acid molecule in to the reference cell. The introduction facilitates the expression or overexpression of a target enzyme. It is understood that the term “facilitates” encompasses the activation of endogenous polynucleotides encoding a target enzyme through genetic modification of e.g., a promoter sequence in a parental microorganism. It is further understood that the term “facilitates” encompasses the introduction of heterologous polynucleotides encoding a target enzyme in to a parental microorganism.

The term “engineer” refers to any manipulation of a microorganism that results in a detectable change in the microorganism, wherein the manipulation includes but is not limited to inserting a polynucleotide and/or polypeptide heterologous to the microorganism and mutating a polynucleotide and/or polypeptide native to the microorganism.

The term “mutation” as used herein indicates any modification of a nucleic acid and/or polypeptide which results in an altered nucleic acid or polypeptide. Mutations include, for example, point mutations, deletions, or insertions of single or multiple residues in a polynucleotide, which includes alterations arising within a protein-encoding region of a gene as well as alterations in regions outside of a protein-encoding sequence, such as, but not limited to, regulatory or promoter sequences. A genetic alteration may be a mutation of any type. For instance, the mutation may constitute a point mutation, a frame-shift mutation, a nonsense mutation, an insertion, or a deletion of part or all of a gene. In addition, in some embodiments of the modified microorganism, a portion of the microorganism genome has been replaced with a heterologous polynucleotide. In some embodiments, the mutations are naturally-occurring. In other embodiments, the mutations are identified and/or enriched through artificial selection pressure. In still other embodiments, the mutations in the microorganism genome are the result of genetic engineering.

The term “biosynthetic pathway”, also referred to as “metabolic pathway”, refers to a set of anabolic or catabolic biochemical reactions for converting one chemical species into another. Gene products belong to the same “metabolic pathway” if they, in parallel or in series, act on the same substrate, produce the same product, or act on or produce a metabolic intermediate (i.e., metabolite) between the same substrate and metabolite end product.

As used herein, the term “isobutanol producing metabolic pathway” refers to an enzyme pathway which produces isobutanol from pyruvate.

The term “NADH-dependent” as used herein with reference to an enzyme, e.g., KARI and/or ADH, refers to an enzyme that catalyzes the reduction of a substrate coupled to the oxidation of NADH with a catalytic efficiency that is greater than the reduction of the same substrate coupled to the oxidation of NADPH at equal substrate and cofactor concentrations.

The term “exogenous” as used herein with reference to various molecules, e.g., polynucleotides, polypeptides, enzymes, etc., refers to molecules that are not normally or naturally found in and/or produced by a given yeast, bacterium, organism, microorganism, or cell in nature.

On the other hand, the term “endogenous” or “native” as used herein with reference to various molecules, e.g., polynucleotides, polypeptides, enzymes, etc., refers to molecules that are normally or naturally found in and/or produced by a given yeast, bacterium, organism, microorganism, or cell in nature.

The term “heterologous” as used herein in the context of a modified host cell refers to various molecules, e.g., polynucleotides, polypeptides, enzymes, etc., wherein at least one of the following is true: (a) the molecule(s) is/are foreign (“exogenous”) to (i.e., not naturally found in) the host cell; (b) the molecule(s) is/are naturally found in (e.g., is “endogenous to”) a given host microorganism or host cell but is either produced in an unnatural location or in an unnatural amount in the cell; and/or (c) the molecule(s) differ(s) in nucleotide or amino acid sequence from the endogenous nucleotide or amino acid sequence(s) such that the molecule differing in nucleotide or amino acid sequence from the endogenous nucleotide or amino acid as found endogenously is produced in an unnatural (e.g., greater than naturally found) amount in the cell.

The term “feedstock” is defined as a raw material or mixture of raw materials supplied to a microorganism or fermentation process from which other products can be made. For example, a carbon source, such as biomass or the carbon compounds derived from biomass are a feedstock for a microorganism that produces a biofuel in a fermentation process. However, a feedstock may contain nutrients other than a carbon source.

The term “substrate” or “suitable substrate” refers to any substance or compound that is converted or meant to be converted into another compound by the action of an enzyme. The term includes not only a single compound, but also combinations of compounds, such as solutions, mixtures and other materials which contain at least one substrate, or derivatives thereof. Further, the term “substrate” encompasses not only compounds that provide a carbon source suitable for use as a starting material, such as any biomass derived sugar, but also intermediate and end product metabolites used in a pathway associated with a recombinant microorganism as described herein.

The term “fermentation” or “fermentation process” is defined as a process in which a microorganism is cultivated in a culture medium containing raw materials, such as feedstock and nutrients, wherein the microorganism converts raw materials, such as a feedstock, into products.

The term “volumetric productivity” or “production rate” is defined as the amount of product formed per volume of medium per unit of time. Volumetric productivity is reported in gram per liter per hour (g/L/h).

The term “specific productivity” or “specific production rate” is defined as the amount of product formed per volume of medium per unit of time per amount of cells. Specific productivity is reported in gram or milligram per liter per hour per OD (g/L/h/OD).

The term “yield” is defined as the amount of product obtained per unit weight of raw material and may be expressed as g product per g substrate (g/g). Yield may be expressed as a percentage of the theoretical yield. “Theoretical yield” is defined as the maximum amount of product that can be generated per a given amount of substrate as dictated by the stoichiometry of the metabolic pathway used to make the product. For example, the theoretical yield for one typical conversion of glucose to isobutanol is 0.41 g/g. As such, a yield of isobutanol from glucose of 0.39 g/g would be expressed as 95% of theoretical or 95% theoretical yield.

The term “titer” is defined as the strength of a solution or the concentration of a substance in solution. For example, the titer of a biofuel in a fermentation broth is described as g of biofuel in solution per liter of fermentation broth (g/L).

“Aerobic conditions” are defined as conditions under which the oxygen concentration in the fermentation medium is sufficiently high for an aerobic or facultative anaerobic microorganism to use as a terminal electron acceptor.

In contrast, “anaerobic conditions” are defined as conditions under which the oxygen concentration in the fermentation medium is too low for the microorganism to use as a terminal electron acceptor. Anaerobic conditions may be achieved by sparging a fermentation medium with an inert gas such as nitrogen until oxygen is no longer available to the microorganism as a terminal electron acceptor. Alternatively, anaerobic conditions may be achieved by the microorganism consuming the available oxygen of the fermentation until oxygen is unavailable to the microorganism as a terminal electron acceptor. Methods for the production of isobutanol under anaerobic conditions are described in commonly owned and co-pending publication, US 2010/0143997, the disclosures of which are herein incorporated by reference in its entirety for all purposes.

“Aerobic metabolism” refers to a biochemical process in which oxygen is used as a terminal electron acceptor to make energy, typically in the form of ATP, from carbohydrates. Aerobic metabolism occurs e.g. via glycolysis and the TCA cycle, wherein a single glucose molecule is metabolized completely into carbon dioxide in the presence of oxygen.

In contrast, “anaerobic metabolism” refers to a biochemical process in which oxygen is not the final acceptor of electrons contained in NADH. Anaerobic metabolism can be divided into anaerobic respiration, in which compounds other than oxygen serve as the terminal electron acceptor, and substrate level phosphorylation, in which the electrons from NADH are utilized to generate a reduced product via a “fermentative pathway.”

In “fermentative pathways”, NAD(P)H donates its electrons to a molecule produced by the same metabolic pathway that produced the electrons carried in NAD(P)H. For example, in one of the fermentative pathways of certain yeast strains, NAD(P)H generated through glycolysis transfers its electrons to pyruvate, yielding ethanol. Fermentative pathways are usually active under anaerobic conditions but may also occur under aerobic conditions, under conditions where NADH is not fully oxidized via the respiratory chain. For example, above certain glucose concentrations, Crabtree positive yeasts produce large amounts of ethanol under aerobic conditions.

The term “byproduct” or “by-product” means an undesired product related to the production of an amino acid, amino acid precursor, chemical, chemical precursor, biofuel, or biofuel precursor.

The term “substantially free” when used in reference to the presence or absence of a protein activity (3-KAR enzymatic activity, ALDH enzymatic activity, PDC enzymatic activity, GPD enzymatic activity, etc.) means the level of the protein is substantially less than that of the same protein in the wild-type host, wherein less than about 50% of the wild-type level is preferred and less than about 30% is more preferred. The activity may be less than about 20%, less than about 10%, less than about 5%, or less than about 1% of wild-type activity. Microorganisms which are “substantially free” of a particular protein activity (3-KAR enzymatic activity, ALDH enzymatic activity, PDC enzymatic activity, GPD enzymatic activity, etc.) may be created through recombinant means or identified in nature.

The term “non-fermenting yeast” is a yeast species that fails to demonstrate an anaerobic metabolism in which the electrons from NADH are utilized to generate a reduced product via a fermentative pathway such as the production of ethanol and CO₂ from glucose. Non-fermentative yeast can be identified by the “Durham Tube Test” (J. A. Barnett, R. W. Payne, and D. Yarrow. 2000. Yeasts Characteristics and Identification. 3^{rd} edition. p. 28-29. Cambridge University Press, Cambridge, UK) or by monitoring the production of fermentation productions such as ethanol and CO₂.

The term “polynucleotide” is used herein interchangeably with the term “nucleic acid” and refers to an organic polymer composed of two or more monomers including nucleotides, nucleosides or analogs thereof, including but not limited to single stranded or double stranded, sense or antisense deoxyribonucleic acid (DNA) of any length and, where appropriate, single stranded or double stranded, sense or antisense ribonucleic acid (RNA) of any length, including siRNA. The term “nucleotide” refers to any of several compounds that consist of a ribose or deoxyribose sugar joined to a purine or a pyrimidine base and to a phosphate group, and that are the basic structural units of nucleic acids. The term “nucleoside” refers to a compound (as guanosine or adenosine) that consists of a purine or pyrimidine base combined with deoxyribose or ribose and is found especially in nucleic acids. The term “nucleotide analog” or “nucleoside analog” refers, respectively, to a nucleotide or nucleoside in which one or more individual atoms have been replaced with a different atom or with a different functional group. Accordingly, the term polynucleotide includes nucleic acids of any length, DNA, RNA, analogs and fragments thereof. A polynucleotide of three or more nucleotides is also called nucleotidic oligomer or oligonucleotide.

It is understood that the polynucleotides described herein include “genes” and that the nucleic acid molecules described herein include “vectors” or “plasmids.” Accordingly, the term “gene”, also called a “structural gene” refers to a polynucleotide that codes for a particular sequence of amino acids, which comprise all or part of one or more proteins or enzymes, and may include regulatory (non-transcribed) DNA sequences, such as promoter sequences, which determine for example the conditions under which the gene is expressed. The transcribed region of the gene may include untranslated regions, including introns, 5′-untranslated region (UTR), and 3′-UTR, as well as the coding sequence.

The term “operon” refers to two or more genes which are transcribed as a single transcriptional unit from a common promoter. In some embodiments, the genes comprising the operon are contiguous genes. It is understood that transcription of an entire operon can be modified (i.e., increased, decreased, or eliminated) by modifying the common promoter. Alternatively, any gene or combination of genes in an operon can be modified to alter the function or activity of the encoded polypeptide. The modification can result in an increase in the activity of the encoded polypeptide. Further, the modification can impart new activities on the encoded polypeptide. Exemplary new activities include the use of alternative substrates and/or the ability to function in alternative environmental conditions.

A “vector” is any means by which a nucleic acid can be propagated and/or transferred between organisms, cells, or cellular components. Vectors include viruses, bacteriophage, pro-viruses, plasmids, phagemids, transposons, and artificial chromosomes such as YACs (yeast artificial chromosomes), BACs (bacterial artificial chromosomes), and PLACs (plant artificial chromosomes), and the like, that are “episomes,” that is, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that are not episomal in nature, or it can be an organism which comprises one or more of the above polynucleotide constructs such as an agrobacterium or a bacterium.

“Transformation” refers to the process by which a vector is introduced into a host cell. Transformation (or transduction, or transfection), can be achieved by any one of a number of means including chemical transformation (e.g. lithium acetate transformation), electroporation, microinjection, biolistics (or particle bombardment-mediated delivery), or agrobacterium mediated transformation.

The term “enzyme” as used herein refers to any substance that catalyzes or promotes one or more chemical or biochemical reactions, which usually includes enzymes totally or partially composed of a polypeptide, but can include enzymes composed of a different molecule including polynucleotides.

The term “protein,” “peptide,” or “polypeptide” as used herein indicates an organic polymer composed of two or more amino acidic monomers and/or analogs thereof. As used herein, the term “amino acid” or “amino acidic monomer” refers to any natural and/or synthetic amino acids including glycine and both D or L optical isomers. The term “amino acid analog” refers to an amino acid in which one or more individual atoms have been replaced, either with a different atom, or with a different functional group. Accordingly, the term polypeptide includes amino acidic polymer of any length including full length proteins, and peptides as well as analogs and fragments thereof. A polypeptide of three or more amino acids is also called a protein oligomer or oligopeptide

The term “homolog,” used with respect to an original polynucleotide or polypeptide of a first family or species, refers to distinct polynucleotides or polypeptides of a second family or species which are determined by functional, structural or genomic analyses to be a polynucleotide or polypeptide of the second family or species which corresponds to the original polynucleotide or polypeptide of the first family or species. Most often, homologs will have functional, structural or genomic similarities. Techniques are known by which homologs of a polynucleotide or polypeptide can readily be cloned using genetic probes and PCR. Identity of cloned sequences as homolog can be confirmed using functional assays and/or by genomic mapping of the genes.

A polypeptide has “homology” or is “homologous” to a second polypeptide if the amino acid sequence encoded by a gene has a similar amino acid sequence to that of the second gene. Alternatively, a polypeptide has homology to a second polypeptide if the two polypeptides have “similar” amino acid sequences. (Thus, the terms “homologous polypeptides” or “homologous proteins” are defined to mean that the two polypeptides have similar amino acid sequences).

The term “analog” or “analogous” refers to polynucleotide or polypeptide sequences that are related to one another in function only and are not from common descent or do not share a common ancestral sequence. Analogs may differ in sequence but may share a similar structure, due to convergent evolution. For example, two enzymes are analogs or analogous if the enzymes catalyze the same reaction of conversion of a substrate to a product, are unrelated in sequence, and irrespective of whether the two enzymes are related in structure.

### Isobutanol Producing Recombinant Microorganisms

A variety of microorganisms convert sugars to produce pyruvate, which is then utilized in a number of pathways of cellular metabolism. In recent years, microorganisms, including yeast, have been engineered to produce a number of desirable products via pyruvate-driven biosynthetic pathways, including isobutanol, an important commodity chemical and biofuel candidate (See, e.g., commonly owned and co-pending patent publications, US 2009/0226991, US 2010/0143997, US 2011/0020889, US 2011/0076733, and WO 2010/075504).

As described herein, the present invention relates to recombinant microorganisms for producing isobutanol, wherein said recombinant microorganisms comprise an isobutanol producing metabolic pathway. In one embodiment, the isobutanol producing metabolic pathway to convert pyruvate to isobutanol can be comprised of the following reactions:

1. 2 pyruvate→acetolactate+CO₂

2. acetolactate+NAD(P)H→2,3-dihydroxyisovalerate+NAD(P)⁺

3. 2,3-dihydroxyisovalerate→alpha-ketoisovalerate

4. alpha-ketoisovalerate→isobutyraldehyde+CO₂

5. isobutyraldehyde+NAD(P)H→isobutanol+NADP

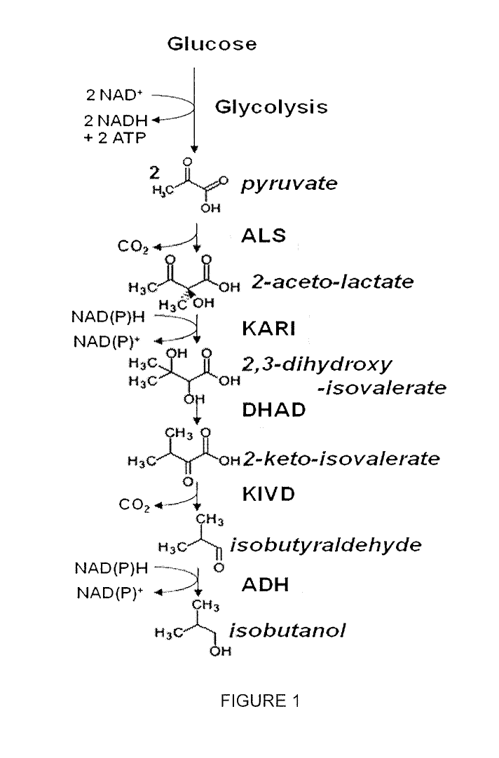

In one embodiment, these reactions are carried out by the enzymes 1) Acetolactate synthase (ALS), 2) Ketol-acid reductoisomerase (KARI), 3) Dihydroxy-acid dehydratase (DHAD), 4) 2-keto-acid decarboxylase, e.g., Keto-isovalerate decarboxylase (KIVD), and 5) an Alcohol dehydrogenase (ADH) (FIG. 1). In some embodiments, the recombinant microorganism may be engineered to overexpress one or more of these enzymes. In an exemplary embodiment, the recombinant microorganism is engineered to overexpress all of these enzymes.

Alternative pathways for the production of isobutanol in yeast have been described in W0/2007/050671 and in Dickinson et al., 1998, J Biol Chem 273:25751-6. These and other isobutanol producing metabolic pathways are within the scope of the present application. In one embodiment, the isobutanol producing metabolic pathway comprises five substrate to product reactions. In another embodiment, the isobutanol producing metabolic pathway comprises six substrate to product reactions. In yet another embodiment, the isobutanol producing metabolic pathway comprises seven substrate to product reactions.

In various embodiments described herein, the recombinant microorganism comprises an isobutanol producing metabolic pathway. In one embodiment, the isobutanol producing metabolic pathway comprises at least one exogenous gene encoding a polypeptide that catalyzes a step in the conversion of pyruvate to isobutanol. In another embodiment, the isobutanol producing metabolic pathway comprises at least two exogenous genes encoding polypeptides that catalyze steps in the conversion of pyruvate to isobutanol. In yet another embodiment, the isobutanol producing metabolic pathway comprises at least three exogenous genes encoding polypeptides that catalyze steps in the conversion of pyruvate to isobutanol. In yet another embodiment, the isobutanol producing metabolic pathway comprises at least four exogenous genes encoding polypeptides that catalyze steps in the conversion of pyruvate to isobutanol. In yet another embodiment, the isobutanol producing metabolic pathway comprises at least five exogenous genes encoding polypeptides that catalyze steps in the conversion of pyruvate to isobutanol. In yet another embodiment, all of the isobutanol producing metabolic pathway steps in the conversion of pyruvate to isobutanol are converted by exogenously encoded enzymes.

In one embodiment, one or more of the isobutanol pathway genes encodes an enzyme that is localized to the cytosol. In one embodiment, the recombinant microorganisms comprise an isobutanol producing metabolic pathway with at least one isobutanol pathway enzyme localized in the cytosol. In another embodiment, the recombinant microorganisms comprise an isobutanol producing metabolic pathway with at least two isobutanol pathway enzymes localized in the cytosol. In yet another embodiment, the recombinant microorganisms comprise an isobutanol producing metabolic pathway with at least three isobutanol pathway enzymes localized in the cytosol. In yet another embodiment, the recombinant microorganisms comprise an isobutanol producing metabolic pathway with at least four isobutanol pathway enzymes localized in the cytosol. In an exemplary embodiment, the recombinant microorganisms comprise an isobutanol producing metabolic pathway with five isobutanol pathway enzymes localized in the cytosol. In yet another exemplary embodiment, the recombinant microorganisms comprise an isobutanol producing metabolic pathway with all isobutanol pathway enzymes localized in the cytosol. Isobutanol producing metabolic pathways in which one or more genes are localized to the cytosol are described in commonly owned and co-pending publication, US 2011/0076733, which is herein incorporated by reference in its entirety for all purposes.

As is understood in the art, a variety of organisms can serve as sources for the isobutanol pathway enzymes, including, but not limited to, Saccharomyces spp., including S. cerevisiae and S. uvarum, Kluyveromyces spp., including K. thermotolerans, K. lactis, and K. marxianus, Pichia spp., Hansenula spp., including H. polymorphs, Candida spp., Trichosporon spp., Yamadazyma spp., including Y. spp. stipitis, Torulaspora pretoriensis, Issatchenkia orientalis, Schizosaccharomyces spp., including S. pombe, Cryptococcus spp., Aspergillus spp., Neurospora spp., or Ustilago spp. Sources of genes from anaerobic fungi include, but not limited to, Piromyces spp., Orpinomyces spp., or Neocallimastix spp. Sources of prokaryotic enzymes that are useful include, but not limited to, Escherichia spp., Zymomonas spp., Staphylococcus spp., Bacillus spp., Clostridium spp., Corynebacterium spp., Pseudomonas spp., Lactococcus spp., Enterobacter spp., Streptococcus spp., Salmonella spp., Slackia spp., Cryptobacterium spp., and Eggerthella spp.

In some embodiments, one or more of these enzymes can be encoded by native genes. Alternatively, one or more of these enzymes can be encoded by heterologous genes.

For example, acetolactate synthases capable of converting pyruvate to acetolactate may be derived from a variety of sources (e.g., bacterial, yeast, Archaea, etc.), including B. subtilis (GenBank Accession No. Q04789.3), L. lactis (GenBank Accession No. NP_{—}267340.1), S. mutans (GenBank Accession No. NP_{—}721805.1), K. pneumoniae (GenBank Accession No. ZP_{—}06014957.1), C. glutamicum (GenBank Accession No. P42463.1), E. cloacae (GenBank Accession No. YP_{—}003613611.1), M. maripaludis (GenBank Accession No. ABX01060.1), M. grisea (GenBank Accession No. AAB81248.1), T. stipitatus (GenBank Accession No. XP_{—}002485976.1), or S. cerevisiae ILV2 (GenBank Accession No. NP_{—}013826.1). Additional acetolactate synthases capable of converting pyruvate to acetolactate are described in commonly owned and co-pending US Publication No. 2011/0076733, which is herein incorporated by reference in its entirety. A review article characterizing the biosynthesis of acetolactate from pyruvate via the activity of acetolactate synthases is provided by Chipman et al., 1998, Biochimica et Biophysica Acta 1385: 401-19, which is herein incorporated by reference in its entirety. Chipman et al. provide an alignment and consensus for the sequences of a representative number of acetolactate synthases. Motifs shared in common between the majority of acetolactate synthases include:

(SEQ ID NO: 215)
SGPG(A/C/V)(T/S)N,
(SEQ ID NO: 216)
GX(P/A)GX(V/A/T),
(SEQ ID NO: 217)
GX(Q/G)(T/A)(L/M)G(Y/F/W)(A/G)X(P/G)
(W/A)AX(G/T)(A/V),
and
(SEQ ID NO: 218)
GD(G/A)(G/S/C)F

motifs at amino acid positions corresponding to the 163-169, 240-245, 521-535, and 549-553 residues, respectively, of the S. cerevisiae ILV2. Thus, a protein harboring one or more of these amino acid motifs can generally be expected to exhibit acetolactate synthase activity.

Ketol-acid reductoisomerases capable of converting acetolactate to 2,3-dihydroxyisovalerate may be derived from a variety of sources (e.g., bacterial, yeast, Archaea, etc.), including E. coli (GenBank Accession No. EGB30597.1), L. lactis (GenBank Accession No. YP_{—}003353710.1), S. exigua (GenBank Accession No. ZP_{—}06160130.1), C. curtam (GenBank Accession No. YP_{—}003151266.1), Shewanella sp. (GenBank Accession No. YP_{—}732498.1), V. fischeri (GenBank Accession No. YP_{—}205911.1), M. maripaludis (GenBank Accession No. YP_{—}001097443.1), B. subtilis (GenBank Accession No. CAB14789), S. pombe (GenBank Accession No. NP_{—}001018845), B. thetaiotamicron (GenBank Accession No. NP 810987), or S. cerevisiae ILV5 (GenBank Accession No. NP 013459.1). Additional ketol-acid reductoisomerases capable of converting acetolactate to 2,3-dihydroxyisovalerate are described in commonly owned and co-pending US Publication No. 2011/0076733, which is herein incorporated by reference in its entirety. An alignment and consensus for the sequences of a representative number of ketol-acid reductoisomerases is provided in commonly owned and co-pending US Publication No. 2010/0143997, which is herein incorporated by reference in its entirety. Motifs shared in common between the majority of ketol-acid reductoisomerases include:

(SEQ ID NO: 219)
G(Y/C/W)GXQ(G/A),
(SEQ ID NO: 220)
(F/Y/L)(S/A)HG(F/L),
(SEQ ID NO: 221)
V(V/I/F)(M/L/A)(A/C)PK,
(SEQ ID NO: 222)
D(L/I)XGE(Q/R)XXLXG,
and
(SEQ ID NO: 223)
S(D/N/T)TA(E/Q/R)XG

motifs at amino acid positions corresponding to the 89-94, 175-179, 194-200, 262-272, and 459-465 residues, respectively, of the E. coli ketol-acid reductoisomerase encoded by ilvC. Thus, a protein harboring one or more of these amino acid motifs can generally be expected to exhibit ketol-acid reductoisomerase activity.

To date, all known, naturally existing ketol-acid reductoisomerases are known to use NADPH as a cofactor. In certain embodiments, a ketol-acid reductoisomerase which has been engineered to used NADH as a cofactor may be utilized to mediate the conversion of acetolactate to 2,3-dihydroxyisovalerate. Engineered NADH-dependent KARI enzymes (“NKRs”) and methods of generating such NKRs are disclosed in commonly owned and co-pending US Publication No. 2010/0143997.

In accordance with the invention, any number of mutations can be made to a KARI enzyme, and in a preferred aspect, multiple mutations can be made to a KARI enzyme to result in an increased ability to utilize NADH for the conversion of acetolactate to 2,3-dihydroxyisovalerate. Such mutations include point mutations, frame shift mutations, deletions, and insertions, with one or more (e.g., one, two, three, four, five or more, etc.) point mutations preferred.

Mutations may be introduced into naturally existing KARI enzymes to create NKRs using any methodology known to those skilled in the art. Mutations may be introduced randomly by, for example, conducting a PCR reaction in the presence of manganese as a divalent metal ion cofactor. Alternatively, oligonucleotide directed mutagenesis may be used to create the NKRs which allows for all possible classes of base pair changes at any determined site along the encoding DNA molecule. In general, this technique involves annealing an oligonucleotide complementary (except for one or more mismatches) to a single stranded nucleotide sequence coding for the KARI enzyme of interest. The mismatched oligonucleotide is then extended by DNA polymerase, generating a double-stranded DNA molecule which contains the desired change in sequence in one strand. The changes in sequence can, for example, result in the deletion, substitution, or insertion of an amino acid. The double-stranded polynucleotide can then be inserted into an appropriate expression vector, and a mutant or modified polypeptide can thus be produced. The above-described oligonucleotide directed mutagenesis can, for example, be carried out via PCR.

Dihydroxy acid dehydratases capable of converting 2,3-dihydroxyisovalerate to α-ketoisovalerate may be derived from a variety of sources (e.g., bacterial, yeast, Archaea, etc.), including E. coli (GenBank Accession No. YP_{—}026248.1), L. lactis (GenBank Accession No. NP_{—}267379.1), S. mutans (GenBank Accession No. NP_{—}722414.1), M. stadtmanae (GenBank Accession No. YP_{—}448586.1), M. tractuosa (GenBank Accession No. YP_{—}004053736.1), Eubacterium SCB49 (GenBank Accession No. ZP_{—}01890126.1), G. forsetti (GenBank Accession No. YP_{—}862145.1), Y. lipolytica (GenBank Accession No. XP_{—}502180.2), N. crassa (GenBank Accession No. XP_{—}963045.1), or S. cerevisiae ILV3 (GenBank Accession No. NP_{—}012550.1). Additional dihydroxy acid dehydratases capable of 2,3-dihydroxyisovalerate to α-ketoisovalerate are described in commonly owned and co-pending US Publication No. 2011/0076733. Motifs shared in common between the majority of dihydroxy acid dehydratases include:

(SEQ ID NO: 224)
SLXSRXXIA,
(SEQ ID NO: 225)
CDKXXPG,
(SEQ ID NO: 226)
GXCXGXXTAN,
(SEQ ID NO: 227)
GGSTN,
(SEQ ID NO: 228)
GPXGXPGMRXE,
(SEQ ID NO: 229)
ALXTDGRXSG,
and
(SEQ ID NO: 230)
GHXXPEA

motifs at amino acid positions corresponding to the 93-101, 122-128, 193-202, 276-280, 482-491, 509-518, and 526-532 residues, respectively, of the E. coli dihydroxy acid dehydratase encoded by ilvD. Thus, a protein harboring one or more of these amino acid motifs can generally be expected to exhibit dihydroxy acid dehydratase activity.

Alcohol dehydrogenases capable of converting isobutyraldehyde to isobutanol may be derived from a variety of sources (e.g., bacterial, yeast, Archaea, etc.), including L. lactis (GenBank Accession No. YP_{—}003354381), B. cereus (GenBank Accession No. YP_{—}001374103.1), N. meningitidis (GenBank Accession No. CBA03965.1), S. sanguinis (GenBank Accession No. YP_{—}001035842.1), L. brevis (GenBank Accession No. YP_{—}794451.1), B. thuringiensis (GenBank Accession No. ZP_{—}04101989.1), P. acidilactici (GenBank Accession No. ZP_{—}06197454.1), B. subtilis (GenBank Accession No. EHA31115.1), N. crassa (GenBank Accession No. CAB91241.1) or S. cerevisiae ADH6 (GenBank Accession No. NP 014051.1). Additional alcohol dehydrogenases capable of converting isobutyraldehyde to isobutanol are described in commonly owned and co-pending US Publication Nos. 2011/0076733 and 2011/0201072. Motifs shared in common between the majority of alcohol dehydrogenases include:

(SEQ ID NO: 231)
C(H/G)(T/S)D(L/I)H,
(SEQ ID NO: 232)
GHEXXGXV,
(SEQ ID NO: 233)
(L/V)(Q/K/E)(V/I/K)G(D/Q)(R/H)(V/A),
(SEQ ID NO: 234)
CXXCXXC,
(SEQ ID NO: 235)
(C/A)(A/G/D)(G/A)XT(T/V),
and
(SEQ ID NO: 236)
G(L/A/C)G(G/P)(L/I/V)G

motifs at amino acid positions corresponding to the 39-44, 59-66, 76-82, 91-97, 147-152, and 171-176 residues, respectively, of the L. lactis alcohol dehydrogenase encoded by adhA. Thus, a protein harboring one or more of these amino acid motifs can generally be expected to exhibit alcohol dehydrogenase activity.

In another embodiment, the yeast microorganism may be engineered to have increased ability to convert pyruvate to isobutanol. In one embodiment, the yeast microorganism may be engineered to have increased ability to convert pyruvate to isobutyraldehyde. In another embodiment, the yeast microorganism may be engineered to have increased ability to convert pyruvate to keto-isovalerate. In another embodiment, the yeast microorganism may be engineered to have increased ability to convert pyruvate to 2,3-dihydroxyisovalerate. In another embodiment, the yeast microorganism may be engineered to have increased ability to convert pyruvate to acetolactate.

Furthermore, any of the genes encoding the foregoing enzymes (or any others mentioned herein (or any of the regulatory elements that control or modulate expression thereof)) may be optimized by genetic/protein engineering techniques, such as directed evolution or rational mutagenesis, which are known to those of ordinary skill in the art. Such action allows those of ordinary skill in the art to optimize the enzymes for expression and activity in yeast.

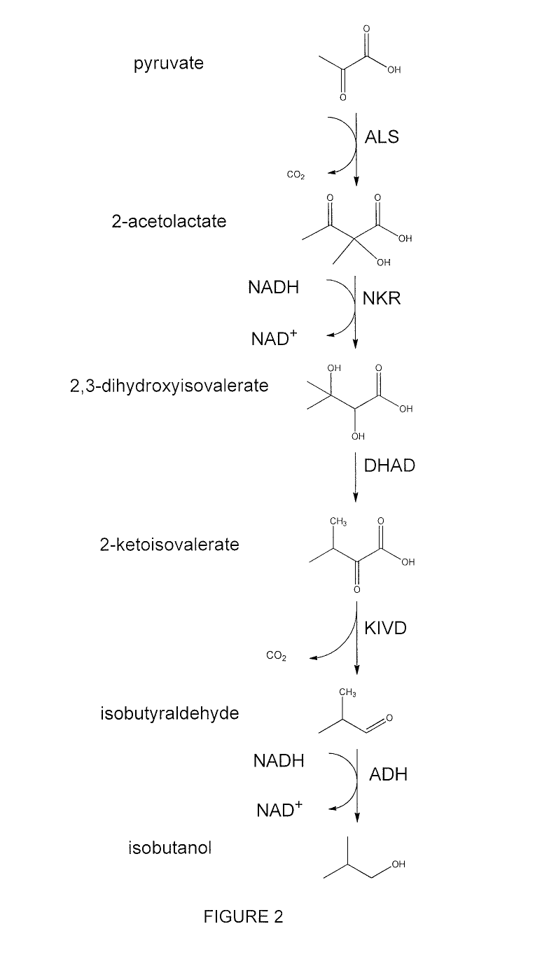

In an exemplary embodiment, pathway steps 2 and 5 of the isobutanol pathway may be carried out by KARI and ADH enzymes that utilize NADH (rather than NADPH) as a cofactor. The present inventors have found that utilization of NADH-dependent KARI (NKR) and ADH enzymes to catalyze pathway steps 2 and 5, respectively, surprisingly enables production of isobutanol at theoretical yield and/or under anaerobic conditions. An example of an NADH-dependent isobutanol pathway is illustrated in FIG. 2. Thus, in one embodiment, the recombinant microorganisms of the present invention may use an NKR to catalyze the conversion of acetolactate to produce 2,3-dihydroxyisovalerate. In another embodiment, the recombinant microorganisms of the present invention may use an NADH-dependent ADH to catalyze the conversion of isobutyraldehyde to produce isobutanol. In yet another embodiment, the recombinant microorganisms of the present invention may use both an NKR to catalyze the conversion of acetolactate to produce 2,3-dihydroxyisovalerate, and an NADH-dependent ADH to catalyze the conversion of isobutyraldehyde to produce isobutanol.

Isobutanol-Producing Metabolic Pathways with Improved KIVD Properties

The fourth step of the isobutanol producing metabolic pathway is catalyzed by a 2-keto acid decarboxylase, e.g., a keto-isovalerate decarboxylase (KIVD), which converts alpha-ketoisovalerate to isobutyraldehyde. 2-keto acid decarboxylases belong to a class of enzymes known as thiamin diphosphate-dependent decarboxylases. The active sites of thiamin diphosphate-dependent decarboxylases are characterized by the presence of two histidine residues, described herein as an “HH”-motif. This HH motif is found at amino acids 112-113 and 114-115 in the L. lactis KivD (SEQ ID NO: 197) and the S. cerevisiae PDC1 (SEQ ID NO: 241), respectively. Thiamin diphosphate-dependent decarboxylases harboring this characteristic HH-motif include pyruvate decarboxylases (PDCs), indolepyruvate decarboxylases (IPDCs), phenylpyruvate decarboxylases (PPDCs), and branched chain 2-keto acid decarboxylases, e.g., keto-isovalerate decarboxylases (KIVDs). Accordingly, the HH-motif is a structural feature that can quickly be used to identify a thiamin-diphosphate-dependent decarboxylase.

The present application relates to the identification of several thiamin diphosphate-dependent decarboxylase enzymes that exhibit high activity for the conversion of alpha-ketoisovalerate to isobutyraldehyde within an isobutanol production pathway. Moreover, the enzymes identified herein have low activity using pyruvate, thereby reducing the conversion of pyruvate—the starting material for many biosynthetic pathways—to the unwanted by-product ethanol in recombinant isobutanol producing microorganisms. Accordingly, this application describes methods of increasing isobutanol production through the use of recombinant microorganisms comprising enzymes with improved properties for the production of isobutanol.

As described herein, the present inventors have identified a KIVD substrate specificity motif “SQFVIMF” (SEQ ID NO: 237) which is generally predictive of: (a) high KIVD activity; (b) reduced PDC activity; and (c) a high KIV/pyruvate activity ratio. This SQFVIMF motif corresponds to the S286, Q377, F381, V461, I465, M538, and F542 residues of the L. lactis KIVD of SEQ ID NO: 197. Because the motif is generally predictive of enzymes exhibiting a high KIV/pyruvate activity ratio, decarboxylases with similarity to this motif are expected to find utility for the conversion of alpha-ketoisovalerate to isobutyraldehyde within an isobutanol production pathway.

Accordingly, one aspect of the application is directed to an isolated nucleic acid molecule encoding a polypeptide with keto-isovalerate decarboxylase (KIVD) activity, wherein said polypeptide comprises at least four of the SQFVIMF specificity residues corresponding to the S286, Q377, F381, V461, I465, M538, and F542 residues of the L. lactis KIVD of SEQ ID NO: 197. Polypeptides with KIVD activity comprising at least four of the SQFVIMF specificity residues are disclosed in the instant application, e.g., at SEQ ID NOs: 1-196. In one embodiment, said polypeptide contains four of the SQFVIMF specificity residues corresponding to the S286, Q377, F381, V461, I465, M538, and F542 residues of the L. lactis KIVD of SEQ ID NO: 197. In another embodiment, said polypeptide contains five of the SQFVIMF specificity residues corresponding to the S286, Q377, F381, V461, I465, M538, and F542 residues of the L. lactis KIVD of SEQ ID NO: 197. In yet another embodiment, said polypeptide contains six of the SQFVIMF specificity residues corresponding to the S286, Q377, F381, V461, I465, M538, and F542 residues of the L. lactis KIVD of SEQ ID NO: 197. In yet another embodiment, said polypeptide contains all seven of the SQFVIMF specificity residues corresponding to the S286, Q377, F381, V461, I465, M538, and F542 residues of the L. lactis KIVD of SEQ ID NO: 197.

As described herein, the present inventors have identified an additional KIVD substrate specificity motif “FTSILFL” (SEQ ID NO: 240) which is generally predictive of: (a) high KIVD activity; (b) reduced PDC activity; and (c) a high KIV/pyruvate activity ratio. This FTSILFL motif corresponds to the F305, T397, S401, I481, L485, F556, and L560 of the F. novicida decarboxylase of SEQ ID NO: 198. Because the motif is generally predictive of enzymes exhibiting a high KIV/pyruvate activity ratio, decarboxylases with similarity to this motif are expected to find utility for the conversion of alpha-ketoisovalerate to isobutyraldehyde within an isobutanol production pathway. Accordingly, another aspect of the application is directed to an isolated nucleic acid molecule encoding a polypeptide with keto-isovalerate decarboxylase (KIVD) activity, wherein said polypeptide comprises at least four of the FTSILFL specificity residues corresponding to the F305, T397, S401, I481, L485, F556, and L560 residues of the F. novicida decarboxylase of SEQ ID NO: 198. Polypeptides with KIVD activity comprising at least four of the FTSILFL specificity residues are disclosed in the instant application, e.g., at SEQ ID NOs: 198-214. In one embodiment, said polypeptide contains four of the FTSILFL specificity residues corresponding to the F305, T397, S401, I481, L485, F556, and L560 residues of the F. novicida decarboxylase of SEQ ID NO: 198. In another embodiment, said polypeptide contains five of the FTSILFL specificity residues corresponding to the F305, T397, S401, I481, L485, F556, and L560 residues of the F. novicida decarboxylase of SEQ ID NO: 198. In yet another embodiment, said polypeptide contains six of the FTSILFL specificity residues corresponding to the F305, T397, S401, I481, L485, F556, and L560 residues of the F. novicida decarboxylase of SEQ ID NO: 198. In yet another embodiment, said polypeptide contains all seven of the FTSILFL specificity residues corresponding to the F305, T397, S401, I481, L485, F556, and L560 residues of the F. novicida decarboxylase of SEQ ID NO: 198.

Another aspect of the application is directed to an isolated nucleic acid molecule encoding a polypeptide with keto-isovalerate decarboxylase (KIVD) activity, wherein said polypeptide is at least about 65% identical to a polypeptide selected from SEQ ID NOs 1-214. Further within the scope of present application are polypeptides with keto-isovalerate decarboxylase (KIVD) activity which are at least about 70%, 75%, 80%, 85%, 90%, 91%, 92%, 93%, 94%, 95%, 96%, 97%, 98%, 99%, or 99.5% identical to a polypeptide selected from SEQ ID NOs 1-214.

In one embodiment, the polypeptide with keto-isovalerate decarboxylase (KIVD) activity is derived from the genus Lactococcus. In a specific embodiment, the polypeptide with keto-isovalerate decarboxylase (KIVD) activity is derived from Lactococcus lactis. In another specific embodiment, the polypeptide with keto-isovalerate decarboxylase (KIVD) activity is selected from SEQ ID NOs: 1-4.

In another embodiment, the polypeptide with keto-isovalerate decarboxylase (KIVD) activity is derived from the genus Melissococcus. In a specific embodiment, the polypeptide with keto-isovalerate decarboxylase (KIVD) activity is derived from Melissococcus plutonius. In another specific embodiment, the polypeptide with keto-isovalerate decarboxylase (KIVD) activity comprises SEQ ID NO: 5.

In yet another embodiment, the polypeptide with keto-isovalerate decarboxylase (KIVD) activity is derived from the genus Listeria. In a specific embodiment, the polypeptide with keto-isovalerate decarboxylase (KIVD) activity is derived from Listeria grayi. In another specific embodiment, the polypeptide with keto-isovalerate decarboxylase (KIVD) activity comprises SEQ ID NO: 6.

In yet another embodiment, the polypeptide with keto-isovalerate decarboxylase (KIVD) activity is derived from a genus selected from Staphylococcus or Macrococcus. In a specific embodiment, the polypeptide with keto-isovalerate decarboxylase (KIVD) activity is derived from Staphylococcus aureus, Staphylococcus epidermidis, Staphylococcus capitis, Staphylococcus haemolyticus, Staphylococcus warneri, Staphylococcus caprae, Staphylococcus saprophyticus, Staphylococcus hominis, Staphylococcus carnosus, Staphylococcus lugdunensis, or Macrococcus caseolyticus. In another specific embodiment, the polypeptide with keto-isovalerate decarboxylase (KIVD) activity is selected from SEQ ID NOs: 7-44.

In yet another embodiment, the polypeptide with keto-isovalerate decarboxylase (KIVD) activity is derived from the genus Staphylococcus. In a specific embodiment, the polypeptide with keto-isovalerate decarboxylase (KIVD) activity is derived from Staphylococcus pseudintermedius. In another specific embodiment, the polypeptide with keto-isovalerate decarboxylase (KIVD) activity is selected from SEQ ID NOs: 45-46.

In yet another embodiment, the polypeptide with keto-isovalerate decarboxylase (KIVD) activity is derived from a genus selected from Bacillus or Clostridium. In a specific embodiment, the polypeptide with keto-isovalerate decarboxylase (KIVD) activity is derived from Bacillus cereus or Clostridium acetobutylicum. In another specific embodiment, the polypeptide with keto-isovalerate decarboxylase (KIVD) activity is selected from SEQ ID NOs: 47-48.

In yet another embodiment, the polypeptide with keto-isovalerate decarboxylase (KIVD) activity is derived from the genus selected Bacillus. In a specific embodiment, the polypeptide with keto-isovalerate decarboxylase (KIVD) activity is derived from Bacillus anthracis, Bacillus cereus, or Bacillus thuringiensis. In another specific embodiment, the polypeptide with keto-isovalerate decarboxylase (KIVD) activity is selected from SEQ ID NOs: 49-90.

In yet another embodiment, the polypeptide with keto-isovalerate decarboxylase (KIVD) activity is derived from a genus selected from the genus Helicobacter. In a specific embodiment, the polypeptide with keto-isovalerate decarboxylase (KIVD) activity is derived from Helicobacter felis or Helicobacter mustelae. In another specific embodiment, the polypeptide with keto-isovalerate decarboxylase (KIVD) activity is selected from SEQ ID NOs: 91-92.

In yet another embodiment, the polypeptide with keto-isovalerate decarboxylase (KIVD) activity is derived from the genus Sarcina. In a specific embodiment, the polypeptide with keto-isovalerate decarboxylase (KIVD) activity is derived from Sarcina ventriculi. In another specific embodiment, the polypeptide with keto-isovalerate decarboxylase (KIVD) activity comprises SEQ ID NO: 93.

In yet another embodiment, the polypeptide with keto-isovalerate decarboxylase (KIVD) activity is derived from the genus Nostoc. In a specific embodiment, the polypeptide with keto-isovalerate decarboxylase (KIVD) activity is derived from Nostoc punctiforme. In another specific embodiment, the polypeptide with keto-isovalerate decarboxylase (KIVD) activity comprises SEQ ID NO: 94.

In yet another embodiment, the polypeptide with keto-isovalerate decarboxylase (KIVD) activity is derived from the genus Salinispora. In a specific embodiment, the polypeptide with keto-isovalerate decarboxylase (KIVD) activity is derived from Salinispora arenicola. In another specific embodiment, the polypeptide with keto-isovalerate decarboxylase (KIVD) activity comprises SEQ ID NO: 95.

In yet another embodiment, the polypeptide with keto-isovalerate decarboxylase (KIVD) activity is derived from the genus Leishmania. In a specific embodiment, the polypeptide with keto-isovalerate decarboxylase (KIVD) activity is derived from Leishmania mexicana, Leishmania major, Leishmania braziliensis, Leishmania donovani, or Leishmania infantum. In another specific embodiment, the polypeptide with keto-isovalerate decarboxylase (KIVD) activity is selected from SEQ ID NOs: 96-100.

In yet another embodiment, the polypeptide with keto-isovalerate decarboxylase (KIVD) activity is derived from an Enterobacteriaceae. In a specific embodiment, the polypeptide with keto-isovalerate decarboxylase (KIVD) activity is derived from Enterobacteriaceae bacterium 9_{—}2_{—}54FAA. In another specific embodiment, the polypeptide with keto-isovalerate decarboxylase (KIVD) activity comprises SEQ ID NO: 101.

In yet another embodiment, the polypeptide with keto-isovalerate decarboxylase (KIVD) activity is derived from a genus selected from Salmonella, Klebsiella, Enterobacter, Cronobacter, or Citrobacter. In a specific embodiment, the polypeptide with keto-isovalerate decarboxylase (KIVD) activity is derived from Salmonella enterica, Klebsiella pneumoniae, Klebsiella veriicola, Klebsiella sp. 1_{—}1_{—}55, Klebsiella sp. MS 92-3, Enterobacter aerogenes, Enterobacter cancerogenus, Enterobacter sp. 638, Enterobacter cloacae, Enterobacter hormaechei, Cronobacter turicensis, or Cronobacter sakazakii. In another specific embodiment, the polypeptide with keto-isovalerate decarboxylase (KIVD) activity is selected from SEQ ID NOs: 102-143.

In yet another embodiment, the polypeptide with keto-isovalerate decarboxylase (KIVD) activity is derived from the genus Pantoea. In a specific embodiment, the polypeptide with keto-isovalerate decarboxylase (KIVD) activity is derived from Pantoea sp. aB, Pantoea ananatis, Pantoea sp. At-9b, Pantoea agglomerans, or Pantoea vagans. In another specific embodiment, the polypeptide with keto-isovalerate decarboxylase (KIVD) activity is selected from SEQ ID NOs: 144-149.

In yet another embodiment, the polypeptide with keto-isovalerate decarboxylase (KIVD) activity is derived from the genus Erwinia. In a specific embodiment, the polypeptide with keto-isovalerate decarboxylase (KIVD) activity is derived from Erwinia amylovora, Erwinia tasmaniensis, Erwinia sp. Ejp617, Erwinia billingiae, or Erwinia pyrifoliae. In another specific embodiment, the polypeptide with keto-isovalerate decarboxylase (KIVD) activity is selected from SEQ ID NOs: 150-155.

In yet another embodiment, the polypeptide with keto-isovalerate decarboxylase (KIVD) activity is derived from the genus Pectobacterium. In a specific embodiment, the polypeptide with keto-isovalerate decarboxylase (KIVD) activity is derived from Pectobacterium carotovorum or Pectobacterium atrosepticum. In another specific embodiment, the polypeptide with keto-isovalerate decarboxylase (KIVD) activity is selected from SEQ ID NOs: 156-158.

In yet another embodiment, the polypeptide with keto-isovalerate decarboxylase (KIVD) activity is derived from the genus Rahnella. In a specific embodiment, the polypeptide with keto-isovalerate decarboxylase (KIVD) activity is derived from Rahnella sp. Y9602. In another specific embodiment, the polypeptide with keto-isovalerate decarboxylase (KIVD) activity comprises SEQ ID NO: 159.

In yet another embodiment, the polypeptide with keto-isovalerate decarboxylase (KIVD) activity is derived from a genus selected from Yersinia, Serratia, or Nasonia. In a specific embodiment, the polypeptide with keto-isovalerate decarboxylase (KIVD) activity is derived from Yersinia aldovae, Yersinia rohdei, Yersinia enterocolitica, Yersinia kristensenii, Yersinia mollaretii, Serratia symbiotica, Serratia sp. AS12, Serratia odorifera, Serratia proteamaculans, or Nasonia vitripennis. In another specific embodiment, the polypeptide with keto-isovalerate decarboxylase (KIVD) activity is selected from SEQ ID NOs: 160-172.

In yet another embodiment, the polypeptide with keto-isovalerate decarboxylase (KIVD) activity is derived from the genus Kineococcus. In a specific embodiment, the polypeptide with keto-isovalerate decarboxylase (KIVD) activity is derived from Kineococcus radiotolerans. In another specific embodiment, the polypeptide with keto-isovalerate decarboxylase (KIVD) activity comprises SEQ ID NO: 173.

In yet another embodiment, the polypeptide with keto-isovalerate decarboxylase (KIVD) activity is derived from the genus Psychrobacter. In a specific embodiment, the polypeptide with keto-isovalerate decarboxylase (KIVD) activity is derived from Psychrobacter arcticus, Psychrobacter cryohalolentis, Psychrobacter sp. PRwf-1, or Psychrobacter sp. 1501. In another specific embodiment, the polypeptide with keto-isovalerate decarboxylase (KIVD) activity is selected from SEQ ID NOs: 174-177.

In yet another embodiment, the polypeptide with keto-isovalerate decarboxylase (KIVD) activity is derived from the genus Corynebacterium. In a specific embodiment, the polypeptide with keto-isovalerate decarboxylase (KIVD) activity is derived from Corynebacterium striatum. In another specific embodiment, the polypeptide with keto-isovalerate decarboxylase (KIVD) activity comprises SEQ ID NO: 178.

In yet another embodiment, the polypeptide with keto-isovalerate decarboxylase (KIVD) activity is derived from the genus Corynebacterium. In a specific embodiment, the polypeptide with keto-isovalerate decarboxylase (KIVD) activity is derived from Corynebacterium kroppenstedtii. In another specific embodiment, the polypeptide with keto-isovalerate decarboxylase (KIVD) activity comprises SEQ ID NO: 179.

In yet another embodiment, the polypeptide with keto-isovalerate decarboxylase (KIVD) activity is derived from the genus Mycobacterium. In a specific embodiment, the polypeptide with keto-isovalerate decarboxylase (KIVD) activity is derived from Mycobacterium testaceum. In another specific embodiment, the polypeptide with keto-isovalerate decarboxylase (KIVD) activity comprises SEQ ID NO: 180.

In yet another embodiment, the polypeptide with keto-isovalerate decarboxylase (KIVD) activity is derived from the genus Nakamurella. In a specific embodiment, the polypeptide with keto-isovalerate decarboxylase (KIVD) activity is derived from Nakamurella multipartita. In another specific embodiment, the polypeptide with keto-isovalerate decarboxylase (KIVD) activity comprises SEQ ID NO: 181.

In yet another embodiment, the polypeptide with keto-isovalerate decarboxylase (KIVD) activity is derived from the genus Segniliparus. In a specific embodiment, the polypeptide with keto-isovalerate decarboxylase (KIVD) activity is derived from Segniliparus rotundus or Sengiliparus rugosus In another specific embodiment, the polypeptide with keto-isovalerate decarboxylase (KIVD) activity is selected from SEQ ID NOs: 182-183.

In yet another embodiment, the polypeptide with keto-isovalerate decarboxylase (KIVD) activity is derived from the genus Mycobacterium. In a specific embodiment, the polypeptide with keto-isovalerate decarboxylase (KIVD) activity is derived from Mycobacterium marinum, Mycobacterium tuberculosis, Mycobacterium avium, Mycobacterium kansasii, Mycobacterium leprae, Mycobacterium parascrofulaceum, Mycobacterium smegmatis, Mycobacterium ulcerans, or Mycobacterium intracellulare. In another specific embodiment, the polypeptide with keto-isovalerate decarboxylase (KIVD) activity is selected from SEQ ID NOs: 184-196.

In yet another embodiment, the application relates to a recombinant microorganism comprising at least one nucleic acid molecule encoding a polypeptide with keto-isovalerate decarboxylase (KIVD) activity, wherein said polypeptide is at least about 65% identical to a polypeptide selected from SEQ ID NOs: 198-208. In a specific embodiment, the polypeptide with keto-isovalerate decarboxylase (KIVD) activity is derived from the genus Francisella. In another specific embodiment, the polypeptide with keto-isovalerate decarboxylase (KIVD) activity is derived from Francisella novicida, Francisella tularensis, or Francisella philomiragia.

In yet another embodiment, the application relates to a recombinant microorganism comprising at least one nucleic acid molecule encoding a polypeptide with keto-isovalerate decarboxylase (KIVD) activity, wherein said polypeptide is at least about 65% identical to SEQ ID NO: 209. In a specific embodiment, the polypeptide with keto-isovalerate decarboxylase (KIVD) activity is derived from the genus Beijerinckia. In another specific embodiment, the polypeptide with keto-isovalerate decarboxylase (KIVD) activity is derived from Beijerinckia indica.

In yet another embodiment, the application relates to a recombinant microorganism comprising at least one nucleic acid molecule encoding a polypeptide with keto-isovalerate decarboxylase (KIVD) activity, wherein said polypeptide is at least about 65% identical to a polypeptide selected from SEQ ID NOs: 210-211. In a specific embodiment, the polypeptide with keto-isovalerate decarboxylase (KIVD) activity is derived from the genus Desulfovibrio.

In yet another embodiment, the application relates to a recombinant microorganism comprising at least one nucleic acid molecule encoding a polypeptide with keto-isovalerate decarboxylase (KIVD) activity, wherein said polypeptide is at least about 65% identical to a polypeptide selected from SEQ ID NOs: 212-213. In a specific embodiment, the polypeptide with keto-isovalerate decarboxylase (KIVD) activity is derived from the genus Edwardsiella. In another specific embodiment, the polypeptide with keto-isovalerate decarboxylase (KIVD) activity is derived from Edwardsiella tarda or Edwardsiella ictaluri.

In yet another embodiment, the application relates to a recombinant microorganism comprising at least one nucleic acid molecule encoding a polypeptide with keto-isovalerate decarboxylase (KIVD) activity, wherein said polypeptide is at least about 65% identical to SEQ ID NO: 214. In a specific embodiment, the polypeptide with keto-isovalerate decarboxylase (KIVD) activity is derived from the genus Singuliasphaera. In another specific embodiment, the polypeptide with keto-isovalerate decarboxylase (KIVD) activity is derived from Singuliasphaera acidiphila.

The invention also includes fragments of the disclosed polypeptides with keto-isovalerate decarboxylase (KIVD) activity which comprise at least 50, 100, 150, 200, 250, 300, 350, 400, 450, 500, 550, or 600 amino acid residues and retain one or more activities associated with keto-isovalerate decarboxylase (KIVD) activity. Such fragments may be obtained by deletion mutation, by recombinant techniques that are routine and well-known in the art, or by enzymatic digestion of the polypeptides of interest using any of a number of well-known proteolytic enzymes. The invention further includes nucleic acid molecules which encode the above described polypeptides and polypeptide fragments exhibiting keto-isovalerate decarboxylase (KIVD) activity.

Another aspect of the application relates to a recombinant microorganism comprising at least one nucleic acid molecule encoding a polypeptide with keto-isovalerate decarboxylase (KIVD) activity, wherein said polypeptide is at least about 65% identical to a polypeptide selected from SEQ ID NOs 1-214. Further within the scope of present application are recombinant microorganisms comprising at least one nucleic acid molecule encoding a polypeptide with keto-isovalerate decarboxylase (KIVD) activity, wherein said polypeptide is at least about 70%, 75%, 80%, 85%, 90%, 91%, 92%, 93%, 94%, 95%, 96%, 97%, 98%, 99%, or 99.5% identical to a polypeptide selected from SEQ ID NOs 1-214.

Isobutanol-Producing Metabolic Pathways with Modified Decarboxylase Enzymes Catalyzing the Conversion of Alpha-Ketoisovalerate to Isobutyraldehyde

As described herein, the present inventors have identified a group of polypeptides with keto-isovalerate decarboxylase (KIVD) activity. One desirable feature of a polypeptide with keto-isovalerate decarboxylase (KIVD) activity is the ability to exhibit high activity for the conversion of alpha-ketoisovalerate to isobutyraldehyde within an isobutanol production pathway. Another desirable property of a polypeptide with keto-isovalerate decarboxylase (KIVD) activity is low activity using pyruvate, thereby reducing the conversion of pyruvate to the unwanted by-product ethanol in recombinant isobutanol producing microorganisms. The present inventors have identified several beneficial mutations which can be made to an existing decarboxylase enzyme to improve the decarboxylase enzyme's ability to catalyze the conversion of alpha-ketoisovalerate to isobutyraldehyde with high specificity.

In one aspect, the application relates to a decarboxylase enzyme which has been modified or mutated to increase the ability of the enzyme to preferentially utilize keto-isovalerate as its substrate. Examples of such decarboxylase enzymes include enzymes having one or more modifications or mutations at positions corresponding to amino acids selected from: (a) aspartic acid 26 of the L. lactis KIVD (SEQ ID NO: 197); (b) histidine 112 of the L. lactis KIVD (SEQ ID NO: 197); (c) histidine 113 of the L. lactis KIVD (SEQ ID NO: 197); (d) glycine 402 of the L. lactis KIVD (SEQ ID NO: 197); and (e) glutamic acid 462 of the L. lactis KIVD (SEQ ID NO: 197). In an exemplary embodiment, the modified decarboxylase enzyme is derived from a corresponding unmodified decarboxylase that is at least about 65%, 70%, 75%, 80%, 85%, 90%, 91%, 92%, 93%, 94%, 95%, 96%, 97%, 98%, 99%, or 99.5% identical to a polypeptide selected from SEQ ID NOs 1-196.

In one specific embodiment, the application is directed to a modified decarboxylase enzyme, wherein the residue corresponding to position 26 of the L. lactis KIVD (SEQ ID NO: 197) is replaced with a residue selected from aspartic acid and glutamic acid. In another specific embodiment, the application is directed to a modified decarboxylase enzyme, wherein the residue corresponding to position 112 of the L. lactis KIVD (SEQ ID NO: 197) is replaced with a residue selected from histidine, arginine, or lysine. In yet another specific embodiment, the application is directed to a modified decarboxylase enzyme, wherein the residue corresponding to position 113 of the L. lactis KIVD (SEQ ID NO: 197) is replaced with a residue selected from histidine, arginine, or lysine. In yet another specific embodiment, the application is directed to a modified decarboxylase enzyme, wherein the residue corresponding to position 402 of the L. lactis KIVD (SEQ ID NO: 197) is replaced with a residue selected from glycine, cysteine, or proline. In yet another specific embodiment, the application is directed to a modified decarboxylase enzyme, wherein the residue corresponding to position 462 of the L. lactis KIVD (SEQ ID NO: 197) is replaced with a residue selected from glutamic acid or aspartic acid.

In another aspect, the application relates to a decarboxylase enzyme which has been modified or mutated to alter one or more substrate-specificity residues. Examples of such decarboxylase enzymes include enzymes having one or more modifications or mutations at positions corresponding to amino acids selected from: (a) serine 286 of the L. lactis KIVD (SEQ ID NO: 197); (b) glutamine 377 of the L. lactis KIVD (SEQ ID NO: 197); (c) phenylalanine 381 of the L. lactis KIVD (SEQ ID NO: 197); (d) valine 461 of the L. lactis KIVD (SEQ ID NO: 197); (e) isoleucine 465 of the L. lactis KIVD (SEQ ID NO: 197); (f) methionine 538 of the L. lactis KIVD (SEQ ID NO: 197); and (g) phenylalanine 542 of the L. lactis KIVD (SEQ ID NO: 197). In an exemplary embodiment, the modified decarboxylase enzyme is derived from a corresponding unmodified decarboxylase that is at least about 65%, 70%, 75%, 80%, 85%, 90%, 91%, 92%, 93%, 94%, 95%, 96%, 97%, 98%, 99%, or 99.5% identical to a polypeptide selected from SEQ ID NOs 1-196.

In one specific embodiment, the application is directed to a modified decarboxylase enzyme, wherein the residue corresponding to position 286 of the L. lactis KIVD (SEQ ID NO: 197) is replaced with a residue selected from serine, threonine, asparagine, glycine, alanine, proline, glutamine, and aspartic acid. In an exemplary embodiment, the residue corresponding to position 286 of the L. lactis KIVD (SEQ ID NO: 197) is replaced with a serine residue. In another specific embodiment, the application is directed to a modified decarboxylase enzyme, wherein the residue corresponding to position 377 of the L. lactis KIVD (SEQ ID NO: 197) is replaced with a residue selected from glutamine, threonine, serine, and asparagine. In an exemplary embodiment, the residue corresponding to position 377 of the L. lactis KIVD (SEQ ID NO: 197) is replaced with a glutamine residue. In yet another specific embodiment, the application is directed to a modified decarboxylase enzyme, wherein the residue corresponding to position 381 of the L. lactis KIVD (SEQ ID NO: 197) is replaced with a residue selected from phenylalanine, alanine, isoleucine, leucine, methionine, tryptophan, tyrosine, and valine. In an exemplary embodiment, the residue corresponding to position 381 of the L. lactis KIVD (SEQ ID NO: 197) is replaced with a phenylalanine residue. In yet another specific embodiment, the application is directed to a modified decarboxylase enzyme, wherein the residue corresponding to position 461 of the L. lactis KIVD (SEQ ID NO: 197) is replaced with a residue selected from valine, phenylalanine, alanine, isoleucine, leucine, methionine, tryptophan, and tyrosine. In an exemplary embodiment, the residue corresponding to position 461 of the L. lactis KIVD (SEQ ID NO: 197) is replaced with a valine residue. In yet another specific embodiment, the application is directed to a modified decarboxylase enzyme, wherein the residue corresponding to position 465 of the L. lactis KIVD (SEQ ID NO: 197) is replaced with a residue selected from isoleucine, valine, phenylalanine, alanine, leucine, methionine, tryptophan, and tyrosine. In an exemplary embodiment, the residue corresponding to position 465 of the L. lactis KIVD (SEQ ID NO: 197) is replaced with an isoleucine residue. In yet another specific embodiment, the application is directed to a modified decarboxylase enzyme, wherein the residue corresponding to position 538 of the L. lactis KIVD (SEQ ID NO: 197) is replaced with a residue selected from methionine, isoleucine, leucine, valine, alanine, cysteine, glycine, phenylalanine, proline, tryptophan, and tyrosine. In an exemplary embodiment, the residue corresponding to position 465 of the L. lactis KIVD (SEQ ID NO: 197) is replaced with a methionine residue. In yet another specific embodiment, the application is directed to a modified decarboxylase enzyme, wherein the residue corresponding to position 542 of the L. lactis KIVD (SEQ ID NO: 197) is replaced with a residue selected from phenylalanine, isoleucine, leucine, methionine, valine, alanine, cysteine, glycine, proline, tryptophan, and tyrosine. In an exemplary embodiment, the residue corresponding to position 542 of the L. lactis KIVD (SEQ ID NO: 197) is replaced with a phenylalanine residue.

In another aspect, the application relates to a decarboxylase enzymes having one or more modifications or mutations at positions corresponding to amino acids selected from: (a) phenylalanine 305 of the F. novicida decarboxylase (SEQ ID NO: 198); (b) threonine 397 of the F. novicida decarboxylase (SEQ ID NO: 198); (c) serine 401 of the F. novicida decarboxylase (SEQ ID NO: 198); (d) isoleucine 481 of the F. novicida decarboxylase (SEQ ID NO: 198); (e) leucine 485 of the F. novicida decarboxylase (SEQ ID NO: 198); (f) phenylalanine 556 of the F. novicida decarboxylase (SEQ ID NO: 198); and (g) leucine 560 of the F. novicida decarboxylase (SEQ ID NO: 198). In an exemplary embodiment, the modified decarboxylase enzyme is derived from a corresponding unmodified decarboxylase that is at least about 65%, 70%, 75%, 80%, 85%, 90%, 91%, 92%, 93%, 94%, 95%, 96%, 97%, 98%, 99%, or 99.5% identical to a polypeptide selected from SEQ ID NOs 1-214.

In one specific embodiment, the application is directed to a modified decarboxylase enzyme, wherein the residue corresponding to position 305 of the F. novicida decarboxylase (SEQ ID NO: 198) is replaced with a residue selected from phenylalanine, tryptophan, histidine, and tyrosine. In an exemplary embodiment, the residue corresponding to position 305 of the F. novicida decarboxylase (SEQ ID NO: 198) is replaced with a phenylalanine residue. In another specific embodiment, the application is directed to a modified decarboxylase enzyme, wherein the residue corresponding to position 397 of the F. novicida decarboxylase (SEQ ID NO: 198) is replaced with a residue selected from threonine, serine, asparagine, and glutamine. In an exemplary embodiment, the residue corresponding to position 397 of the F. novicida decarboxylase (SEQ ID NO: 198) is replaced with a threonine residue. In yet another specific embodiment, the application is directed to a modified decarboxylase enzyme, wherein the residue corresponding to position 401 of the F. novicida decarboxylase (SEQ ID NO: 198) is replaced with a residue selected from serine, threonine, asparagine, and glutamine. In an exemplary embodiment, the residue corresponding to position 401 of the F. novicida decarboxylase (SEQ ID NO: 198) is replaced with a serine residue. In yet another specific embodiment, the application is directed to a modified decarboxylase enzyme, wherein the residue corresponding to position 481 of the F. novicida decarboxylase (SEQ ID NO: 198) is replaced with a residue selected from isoleucine, methionine, leucine, valine, alanine, phenylalanine, tryptophan, and tyrosine. In an exemplary embodiment, the residue corresponding to position 481 of the F. novicida decarboxylase (SEQ ID NO: 198) is replaced with an isoleucine residue. In yet another specific embodiment, the application is directed to a modified decarboxylase enzyme, wherein the residue corresponding to position 485 of the F. novicida decarboxylase (SEQ ID NO: 198) is replaced with a residue selected from leucine, isoleucine, valine, phenylalanine, alanine, methionine, tryptophan, and tyrosine. In an exemplary embodiment, the residue corresponding to position 485 of the F. novicida decarboxylase (SEQ ID NO: 198) is replaced with a leucine residue. In yet another specific embodiment, the application is directed to a modified decarboxylase enzyme, wherein the residue corresponding to position 556 of the F. novicida decarboxylase (SEQ ID NO: 198) is replaced with a residue selected from phenylalanine, methionine, isoleucine, leucine, valine, alanine, cysteine, glycine, proline, tryptophan, and tyrosine. In an exemplary embodiment, the residue corresponding to position 556 of the F. novicida decarboxylase (SEQ ID NO: 198) is replaced with a phenylalanine residue. In yet another specific embodiment, the application is directed to a modified decarboxylase enzyme, wherein the residue corresponding to position 560 of the F. novicida decarboxylase (SEQ ID NO: 198) is replaced with a residue selected from leucine, isoleucine, leucine, methionine, valine, alanine, cysteine, glycine, and proline. In an exemplary embodiment, the residue corresponding to position 560 of the F. novicida decarboxylase (SEQ ID NO: 198) is replaced with a leucine residue.

In another aspect, the application relates to a pyruvate decarboxylase (PDC) enzyme which has been modified or mutated to alter one or more substrate-specificity residues. In an exemplary embodiment, the substrate specificity of said PDC has been altered to prefer α-ketoisovalerate instead of its natively preferred substrate, pyruvate. Accordingly, the present application provides PDC variants with substrate specificity towards α-ketoisovalerate for use in the conversion of α-ketoisovalerate to isobutyraldehyde within the isobutanol biosynthetic pathway.

In certain embodiments, the application relates to pyruvate decarboxylase variants having one or more modifications or mutations at positions corresponding to amino acids selected from: (a) phenylalanine 292 of the S. cerevisiae PDC1 (SEQ ID NO: 241); (b) threonine 388 of the S. cerevisiae PDC1 (SEQ ID NO: 241); (c) alanine 392 of the S. cerevisiae PDC1 (SEQ ID NO: 241); (d) serine 408 of the S. cerevisiae PDC1 (SEQ ID NO: 241); (e) valine 410 of the S. cerevisiae PDC1 (SEQ ID NO: 241); (f) isoleucine 476 of the S. cerevisiae PDC1 (SEQ ID NO: 241); (g) glutamine 552 of the S. cerevisiae PDC1 (SEQ ID NO: 241); and (h) threonine 556 of the S. cerevisiae PDC1 (SEQ ID NO: 241). In an exemplary embodiment, the modified decarboxylase enzyme is derived from a corresponding unmodified decarboxylase that is at least about 65%, 70%, 75%, 80%, 85%, 90%, 91%, 92%, 93%, 94%, 95%, 96%, 97%, 98%, 99%, or 99.5% identical to a wild-type pyruvate decarboxylase. In one embodiment, the wild-type, unmodified pyruvate decarboxylase is obtained from a yeast microorganism. In a further embodiment, the wild-type, unmodified pyruvate decarboxylase is obtained from a yeast microorganism classified into a genera selected from the group consisting of Saccharomyces, Kluyveromyces, Candida, Pichia, Issatchenkia, Debaryomyces, Hansenula, Pachysolen, Yarrowia, Schizosaccharomyces, Tricosporon, Rhodotorula, and Myxozyma. In another further embodiment, the wild-type, unmodified pyruvate decarboxylase is obtained from a Saccharomyces yeast. In an exemplary embodiment, the wild-type, unmodified pyruvate decarboxylase is obtained from Saccharomyces cerevisiae. In another exemplary embodiment, the wild-type, unmodified pyruvate decarboxylase is PDC1 (SEQ ID NO: 241), PDC5 (SEQ ID NO: 242), or PDC6 (SEQ ID NO: 243) of S. cerevisiae. In yet another exemplary embodiment, the wild-type, unmodified pyruvate decarboxylase is selected from SEQ ID NOs: 244-251.

In one specific embodiment, the application is directed to a modified decarboxylase enzyme, wherein the residue corresponding to position 292 of the S. cerevisiae PDC1 (SEQ ID NO: 241) is replaced with a residue selected from serine, threonine, asparagine, glutamine, and tyrosine. In an exemplary embodiment, the residue corresponding to position 292 of the S. cerevisiae PDC1 (SEQ ID NO: 241) is replaced with a serine residue. In another exemplary embodiment, the residue corresponding to position 292 of the S. cerevisiae PDC1 (SEQ ID NO: 241) is replaced with a threonine residue. In another specific embodiment, the application is directed to a modified decarboxylase enzyme, wherein the residue corresponding to position 388 of the S. cerevisiae PDC1 (SEQ ID NO: 241) is replaced with a residue selected from glutamine, threonine, serine, and asparagine. In an exemplary embodiment, the residue corresponding to position 388 of the S. cerevisiae PDC1 (SEQ ID NO: 241) is replaced with a glutamine residue. In yet another specific embodiment, the application is directed to a modified decarboxylase enzyme, wherein the residue corresponding to position 392 of the S. cerevisiae PDC1 (SEQ ID NO: 241) is replaced with a residue selected from serine, phenylalanine, alanine, cysteine, threonine, asparagine, and glutamine. In an exemplary embodiment, the residue corresponding to position 392 of the S. cerevisiae PDC1 (SEQ ID NO: 241) is replaced with a serine residue. In another exemplary embodiment, the residue corresponding to position 392 of the S. cerevisiae PDC1 (SEQ ID NO: 241) is replaced with a residue selected from phenylalanine, cysteine, and alanine. In yet another specific embodiment, the application is directed to a modified decarboxylase enzyme, wherein the residue corresponding to position 408 of the S. cerevisiae PDC1 (SEQ ID NO: 241) is replaced with a residue selected from glycine and serine. In an exemplary embodiment, the residue corresponding to position 408 of the S. cerevisiae PDC1 (SEQ ID NO: 241) is replaced with a glycine residue. In yet another specific embodiment, the application is directed to a modified decarboxylase enzyme, wherein the residue corresponding to position 410 of the S. cerevisiae PDC1 (SEQ ID NO: 241) is replaced with a residue selected from proline and valine. In an exemplary embodiment, the residue corresponding to position 410 of the S. cerevisiae PDC1 (SEQ ID NO: 241) is replaced with a proline residue. In yet another specific embodiment, the application is directed to a modified decarboxylase enzyme, wherein the residue corresponding to position 476 of the S. cerevisiae PDC1 (SEQ ID NO: 241) is replaced with a residue selected from valine, methionine, leucine, alanine, phenylalanine, tryptophan, and tyrosine. In an exemplary embodiment, the residue corresponding to position 476 of the S. cerevisiae PDC1 (SEQ ID NO: 241) is replaced with a valine residue. In yet another specific embodiment, the application is directed to a modified decarboxylase enzyme, wherein the residue corresponding to position 552 of the S. cerevisiae PDC1 (SEQ ID NO: 241) is replaced with a residue selected from methionine, leucine, isoleucine, valine, glutamine, phenylalanine, alanine, tryptophan, and tyrosine. In an exemplary embodiment, the residue corresponding to position 552 of the S. cerevisiae PDC1 (SEQ ID NO: 241) is replaced with a methionine residue. In another exemplary embodiment, the residue corresponding to position 552 of the S. cerevisiae PDC1 (SEQ ID NO: 241) is replaced with a residue selected from leucine, isoleucine, and valine. In yet another specific embodiment, the application is directed to a modified decarboxylase enzyme, wherein the residue corresponding to position 556 of the S. cerevisiae PDC1 (SEQ ID NO: 241) is replaced with a residue selected from isoleucine, phenylalanine, methionine, leucine, valine, threonine, alanine, cysteine, glycine, proline, tryptophan, and tyrosine. In an exemplary embodiment, the residue corresponding to position 556 of the S. cerevisiae PDC1 (SEQ ID NO: 241) is replaced with an isoleucine residue. In another exemplary embodiment, the residue corresponding to position 556 of the S. cerevisiae PDC1 (SEQ ID NO: 241) is replaced with a residue selected from leucine, phenylalanine, and valine.

The positions corresponding to the D26, H112, H113, S286, Q377, F381, V461, E462, I465, M538, and F542 residues of the L. lactis KIVD (SEQ ID NO: 197) may be readily identified for by one of skill in the art for any decarboxylase enzyme, including, but not limited to, those identified herein (e.g., the decarboxylases of SEQ ID NOs: 1-214). Likewise, the positions corresponding to the F305, T397, S401, I481, L485, F556, and L560 residues of the F. novicida decarboxylase (SEQ ID NO: 198) may be readily identified for by one of skill in the art for any decarboxylase enzyme, including, but not limited to, those identified herein (e.g., the decarboxylases of SEQ ID NOs: 1-214). Similarly, the positions corresponding to the F292, T388, A392, S408, V410, 1476, Q552, and T556 residues of the S. cerevisiae PDC1 (SEQ ID NO: 241) may be readily identified for by one of skill in the art for any known pyruvate decarboxylase enzyme. It will be readily apparent to those of skill in the art that the numbering of amino acids in decarboxylases other than SEQ ID NOs: 197, 198, and 241 may be different than that set forth for SEQ ID NOs: 197, 198, and 241, respectively. Corresponding amino acids in other decarboxylases are easily identified by visual inspection of the amino acid sequences or by using commercially available homology software programs. Thus, given the defined regions for changes and the assays described in the present application, one with skill in the art can make one or a number of modifications which would result in an increased ability to specifically catalyze the conversion of alpha-ketoisovalerate to isobutyraldehyde, in any decarboxylase enzyme of interest.

The application also includes fragments of the modified decarboxylase enzymes which comprise at least 50, 100, 150, 200, 250, 300, 350, 400, 450, 500, 550, or 600 amino acid residues and retain one or more activities associated with decarboxylase enzymes. Such fragments may be obtained by deletion mutation, by recombinant techniques that are routine and well-known in the art, or by enzymatic digestion of the decarboxylase enzyme(s) of interest using any of a number of well-known proteolytic enzymes. The invention further includes nucleic acid molecules which encode the above described mutant decarboxylase enzymes and decarboxylase enzyme fragments.

The application also includes modified decarboxylases comprising an amino acid sequence that can be optimally aligned with the corresponding unmodified, wild-type decarboxylase to generate a similarity score which is at least about 50%, more preferably at least about 60%, more preferably at least about 70%, more preferably at least about 80%, more preferably at least about 90%, or most preferably at least about 95% of the score for the reference sequence using the BLOSUM62 matrix, with a gap existence penalty of 11 and a gap extension penalty of 1.

Similarity scores provide a predictive means of attributing conserved function in a variant protein. Importantly, these scores are maximally predictive of conserved function, allowing for coverage of functional sequence variants while more accurately excluding non-functional variants. The exclusion of non-functional variants is best realized using a sequence identifier that is maximally predictive of conserved function, which is satisfied by the similarity score approach. See, e.g., Holman, 21 Santa Clara Computer & High Tech L.J. 55 (2004).

Two sequences are “optimally aligned” when they are aligned for similarity scoring using a defined amino acid substitution matrix (e.g., BLOSUM62), gap existence penalty and gap extension penalty so as to arrive at the highest score possible for that pair of sequences. Amino acid substitution matrices and their use in quantifying the similarity between two sequences are well-known in the art. The BLOSUM62 matrix is often used as a default scoring substitution matrix in sequence alignment protocols such as Gapped BLAST 2.0. The gap existence penalty is imposed for the introduction of a single amino acid gap in one of the aligned sequences, and the gap extension penalty is imposed for each additional empty amino acid position inserted into an already opened gap. The alignment is defined by the amino acids positions of each sequence at which the alignment begins and ends, and optionally by the insertion of a gap or multiple gaps in one or both sequences, so as to arrive at the highest possible score. While optimal alignment and scoring can be accomplished manually, the process is facilitated by the use of a computer-implemented alignment algorithm, e.g., gapped BLAST 2.0, described in Altschul et al, (1997) Nucleic Acids Res. 25:3389-3402, and made available to the public at the National Center for Biotechnology Information Website. Optimal alignments, including multiple alignments, can be prepared using, e.g., PSI-BLAST with no compositional adjustments.

With respect to amino acid sequence that is optimally aligned with a reference sequence (e.g., a wild-type, unmodified decarboxylase sequence), an amino acid residue “corresponds to” the position in the reference sequence with which the residue is paired in the alignment. The position is denoted by a number that sequentially identifies each amino acid in the reference sequence based on its position relative to the N-terminus. For example, in SEQ ID NO: 241, position 1 is M, position 2 is S, position 3 is E, etc. When a test sequence, (e.g., a corresponding modified variant of SEQ ID NO: 241) is optimally aligned to the reference sequence, a residue in the test sequence that aligns with the E at position 3 is said to “correspond to position 3” of SEQ ID NO: 241. Owing to deletions, insertion, truncations, fusions, etc., that must be taken into account when determining an optimal alignment, in general the amino acid residue number in a test sequence as determined by simply counting from the N-terminal will not necessarily be the same as the number of its corresponding position in the reference sequence. For example, in a case where there is a deletion in an aligned test sequence, there will be no amino acid that corresponds to a position in the reference sequence at the site of deletion. Where there is an insertion in an aligned reference sequence, that insertion will not correspond to any amino acid position in the reference sequence. In the case of truncations or fusions there can be stretches of amino acids in either the reference or aligned sequence that do not correspond to any amino acid in the corresponding sequence.

With respect to SEQ ID NO: 241, the highest similarity score achievable is 2903, which represents 100% of the similarity score for the reference sequence using the BLOSUM62 matrix, a gap existence penalty of 11, and a gap extension penalty of 1. Accordingly, similarity scores of 1452, 1742, 2032, 2322, 2613, and 2758 for variants of SEQ ID NO: 241 would represent 50%, 60%, 70%, 80%, 90%, and 95% of the similarity score for the reference sequence, i.e., SEQ ID NO; 241. Similarity scores generally allow for a greater number of relatively conservative substitutions than for example, a sequence identity determination, particularly when the substituted amino acids share similar chemical and structural characteristics. Accordingly, similarity score is a highly predictive tool for discriminating between functional and non-functional sequence variants.

In addition, as is understood by the skilled artisan, not all positions within an enzyme are created equal. Certain “permissive sites” are more likely to accommodate mutations without affecting activity or stability. In a sequence family such as the thiamin diphosphate-dependent decarboxylases, there are hundreds of relatively permissive sites. One method to identify permissive sites is by quantifying the extent to which each site has variable amino acids among a collection of homologs. A standard calculation to quantify this variability is to compute the sequence entropy for each site.

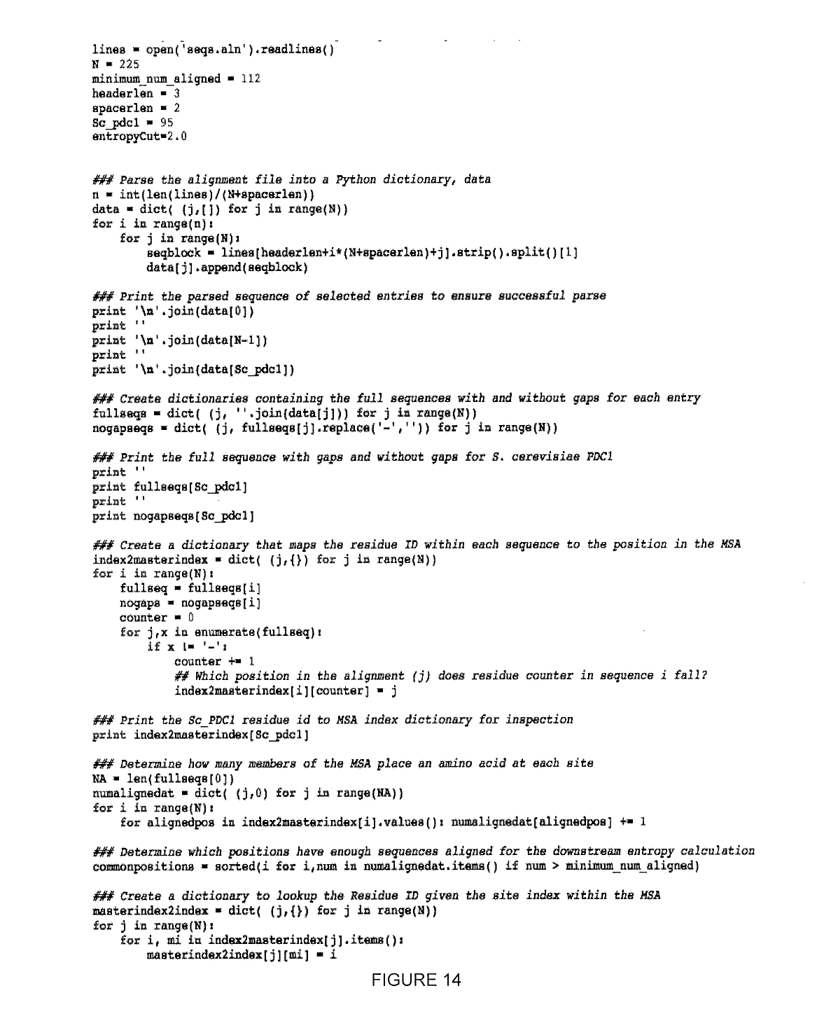

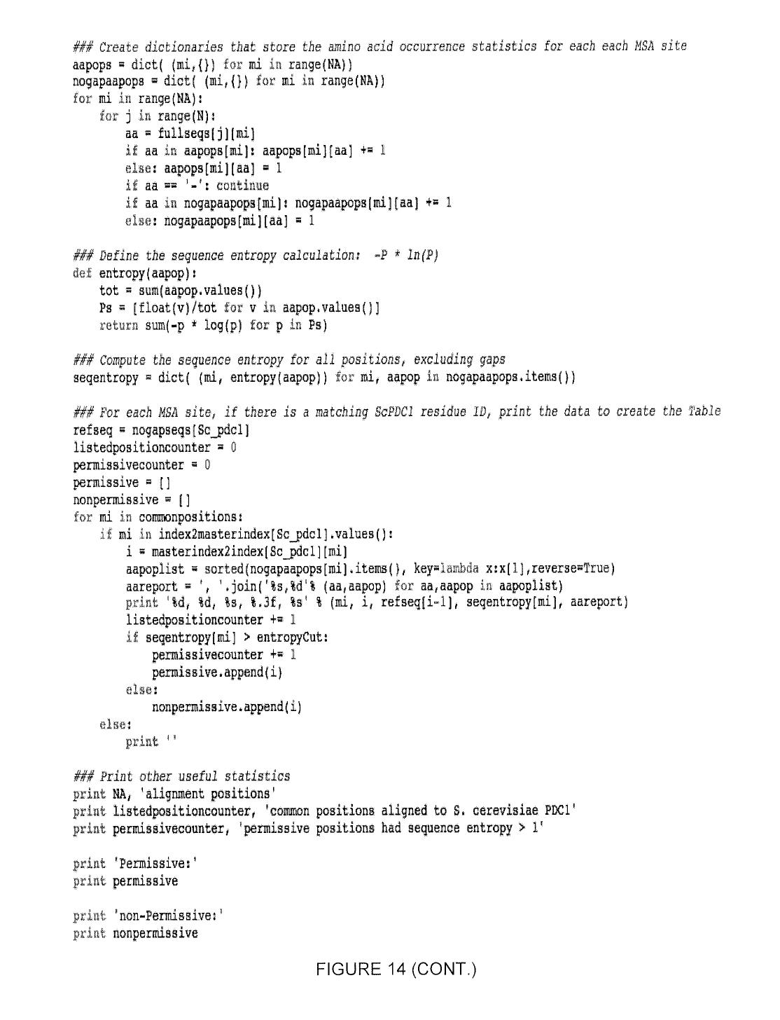

To accomplish this, 225 sequences corresponding to SEQ ID NOs: 1-214 and 241-251 were aligned using CLUSTAL 2.0.12, a standard, well-known software for multiple sequence alignment. These sequences vary in length. Accordingly, the multiple sequence alignment has a number of gaps. Typically, sequence identity is calculated by counting the number of matching amino acids after aligning two sequences, ignoring gaps in the alignment. To proceed, the analysis was limited to positions in the multiple sequence alignment where at least half of the sequences (>112) have an amino acid rather than a gap. Furthermore, for numbering simplicity, only sites for which S. cerevisiae PDC1 (SEQ ID NO: 241) has an amino acid rather than a gap were considered. This results in 553 aligned positions. For each of these aligned positions, the sequence entropy (FIG. 14) was calculated. First, the probability P of observing each amino acid variant found at this site was calculated. Then the sum of −P*ln(P) over all amino acid variants was computed. If the site is completely conserved (for example, the histidine amino acids found in the HH-motif common to all 225 sequences), the sequence entropy is 0. In contrast, if all 20 amino acids were found with equal probability, the sequence entropy would be 3.0.

Several positions within the multiple sequence alignment are quite diverse, with high sequence entropy. Of the 553 positions, 338 have sequence entropy exceeding a threshold of 1.0, 224 also exceed 1.5, 150 also exceed 1.8, and 98 also exceed 2.0. For example, the site for Thr104 from ScPDC1 has sequence entropy of 2.004. At this site, 12 amino acid variants are found, with the most common variants being Thr (74/225), Ser (53/225), Pro (32/225), Cys (28/225), Ala (19/225), and Gly 15/225).

As used herein, a permissive site exceeds a specified sequence entropy threshold using the code illustrated in FIG. 14. Using a threshold level of >1.0 for permissive sites, the following positions corresponding to S. cerevisiae PDC1 residues are relatively permissive sites within the multiple sequence alignment: 1, 2, 3, 4, 5, 7, 8, 11, 15, 16, 17, 19, 20, 21, 22, 32, 36, 38, 39, 40, 41, 42, 43, 44, 49, 64, 65, 67, 71, 82, 92, 96, 97, 101, 103, 104, 105, 106, 107, 108, 109, 111, 112, 113, 121, 123, 124, 126, 127, 129, 130, 131, 134, 136, 137, 138, 141, 142, 146, 147, 154, 155, 156, 157, 158, 159, 160, 166, 169, 172, 173, 174, 175, 176, 177, 178, 179, 180, 181, 182, 183, 184, 185, 186, 189, 190, 191, 192, 193, 194, 195, 196, 197, 198, 199, 200, 201, 202, 203, 204, 205, 206, 207, 209, 210, 211, 212, 213, 214, 215, 216, 220, 221, 222, 223, 225, 226, 227, 228, 229, 230, 232, 233, 234, 235, 236, 237, 238, 239, 240, 242, 244, 246, 247, 251, 252, 253, 255, 256, 258, 260, 262, 264, 266, 267, 269, 270, 271, 272, 273, 274, 275, 278, 281, 282, 284, 285, 287, 288, 289, 292, 293, 299, 300, 301, 302, 303, 304, 305, 306, 308, 309, 310, 311, 312, 313, 314, 315, 316, 317, 318, 319, 320, 321, 322, 323, 324, 325, 327, 328, 329, 331, 332, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 366, 368, 369, 370, 372, 373, 374, 375, 376, 377, 379, 380, 381, 383, 384, 385, 391, 392, 395, 396, 397, 398, 399, 402, 403, 404, 405, 406, 407, 408, 422, 423, 425, 427, 429, 434, 435, 438, 441, 447, 451, 454, 456, 457, 458, 460, 461, 462, 463, 465, 467, 469, 472, 479, 483, 484, 485, 486, 491, 492, 494, 496, 497, 500, 501, 503, 504, 505, 507, 508, 509, 510, 511, 513, 514, 515, 516, 517, 519, 520, 521, 522, 523, 525, 526, 527, 528, 529, 530, 531, 532, 533, 534, 535, 539, 540, 541, 542, 543, 545, 547, 548, 550, 551, 552, 553, 554, 555, 556, 557, 558, 559, 561, 562.

In contrast, sites below a specified sequence entropy threshold can be used to identify relatively non-permissive sites. Accordingly, as used herein, a non-permissive sitefalls below a specified threshold using the code illustrated in FIG. 14. Using a threshold level of <1.0 for non-permissive sites, the following positions corresponding to S. cerevisiae PDC1 residues are relatively non-permissive sites within the multiple sequence alignment: 6, 9, 10, 12, 13, 14, 18, 23, 24, 25, 26, 27, 28, 29, 30, 31, 33, 34, 35, 37, 45, 46, 47, 48, 50, 51, 52, 53, 54, 55, 56, 57, 58, 59, 60, 61, 62, 63, 66, 68, 69, 70, 72, 73, 74, 75, 76, 77, 78, 79, 80, 81, 83, 84, 85, 86, 87, 88, 89, 90, 91, 93, 94, 95, 98, 99, 100, 102, 110, 114, 115, 116, 117, 118, 119, 120, 122, 125, 128, 132, 133, 135, 139, 140, 143, 145, 148, 149, 150, 151, 152, 153, 161, 162, 163, 164, 165, 167, 168, 170, 171, 208, 217, 218, 219, 224, 231, 241, 243, 245, 248, 249, 250, 254, 257, 259, 261, 263, 265, 268, 276, 277, 279, 280, 283, 286, 290, 291, 294, 295, 296, 297, 298, 307, 326, 330, 333, 365, 367, 371, 378, 382, 386, 387, 388, 389, 390, 393, 394, 400, 401, 409, 410, 411, 412, 413, 414, 415, 416, 417, 418, 419, 420, 421, 424, 426, 428, 436, 437, 439, 440, 442, 443, 444, 445, 446, 448, 449, 450, 452, 453, 455, 459, 464, 466, 468, 470, 471, 473, 474, 475, 476, 477, 478, 480, 481, 482, 487, 488, 489, 490, 493, 495, 498, 499, 502, 512, 518, 536, 537, 538, 544, 546, 549, 560.

In certain embodiments, the threshold level may be set at 1.8. Using a threshold level of >1.8 for permissive sites, the following positions corresponding to S. cerevisiae PDC1 residues are relatively permissive sites within the multiple sequence alignment: 1, 2, 3, 15, 20, 42, 44, 103, 104, 105, 108, 109, 123, 126, 138, 146, 147, 154, 158, 166, 173, 174, 177, 178, 180, 181, 182, 183, 184, 185, 186, 189, 190, 191, 192, 194, 195, 198, 199, 201, 202, 203, 205, 206, 207, 209, 210, 213, 223, 228, 229, 230, 232, 233, 237, 239, 255, 258, 260, 264, 266, 269, 270, 271, 274, 275, 281, 300, 302, 303, 312, 313, 317, 319, 320, 322, 325, 327, 328, 331, 332, 334, 335, 336, 337, 338, 339, 340, 341, 342, 343, 344, 345, 347, 348, 349, 350, 351, 352, 353, 354, 355, 356, 359, 360, 361, 362, 363, 364, 368, 369, 372, 373, 376, 397, 399, 402, 405, 429, 435, 483, 484, 492, 497, 500, 504, 507, 508, 510, 513, 515, 516, 519, 523, 526, 527, 528, 529, 530, 532, 534, 543, 545, 547, 550, 551, 553, 557, 558, 562. Likewise, using a threshold level of <1.8 for non-permissive sites, the following positions corresponding to S. cerevisiae PDC1 residues are relatively non-permissive sites within the multiple sequence alignment: 4, 5, 6, 7, 8, 9, 10, 11, 12, 13, 14, 16, 17, 18, 19, 21, 22, 23, 24, 25, 26, 27, 28, 29, 30, 31, 32, 33, 34, 35, 36, 37, 38, 39, 40, 41, 43, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 106, 107, 110, 111, 112, 113, 114, 115, 116, 117, 118, 119, 120, 121, 122, 124, 125, 127, 128, 129, 130, 131, 132, 133, 134, 135, 136, 137, 139, 140, 141, 142, 143, 145, 148, 149, 150, 151, 152, 153, 155, 156, 157, 159, 160, 161, 162, 163, 164, 165, 167, 168, 169, 170, 171, 172, 175, 176, 179, 193, 196, 197, 200, 204, 208, 211, 212, 214, 215, 216, 217, 218, 219, 220, 221, 222, 224, 225, 226, 227, 231, 234, 235, 236, 238, 240, 241, 242, 243, 244, 245, 246, 247, 248, 249, 250, 251, 252, 253, 254, 256, 257, 259, 261, 262, 263, 265, 267, 268, 272, 273, 276, 277, 278, 279, 280, 282, 283, 284, 285, 286, 287, 288, 289, 290, 291, 292, 293, 294, 295, 296, 297, 298, 299, 301, 304, 305, 306, 307, 308, 309, 310, 311, 314, 315, 316, 318, 321, 323, 324, 326, 329, 330, 333, 346, 357, 358, 365, 366, 367, 370, 371, 374, 375, 377, 378, 379, 380, 381, 382, 383, 384, 385, 386, 387, 388, 389, 390, 391, 392, 393, 394, 395, 396, 398, 400, 401, 403, 404, 406, 407, 408, 409, 410, 411, 412, 413, 414, 415, 416, 417, 418, 419, 420, 421, 422, 423, 424, 425, 426, 427, 428, 434, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 485, 486, 487, 488, 489, 490, 491, 493, 494, 495, 496, 498, 499, 501, 502, 503, 505, 509, 511, 512, 514, 517, 518, 520, 521, 522, 525, 531, 533, 535, 536, 537, 538, 539, 540, 541, 542, 544, 546, 548, 549, 552, 554, 555, 556, 559, 560, 561.

In certain embodiments, the threshold level may be set at 2.0. Using a threshold level of >2.0 for permissive sites, the following positions corresponding to S. cerevisiae PDC1 residues are relatively permissive sites within the multiple sequence alignment: 1, 2, 3, 15, 20, 42, 44, 104, 105, 108, 123, 126, 138, 147, 154, 158, 166, 173, 174, 177, 178, 180, 181, 184, 185, 186, 189, 190, 191, 192, 194, 195, 198, 202, 205, 209, 210, 223, 228, 229, 230, 232, 239, 255, 266, 271, 303, 313, 319, 320, 322, 325, 327, 331, 334, 335, 336, 338, 339, 340, 342, 343, 344, 345, 347, 348, 349, 350, 351, 352, 354, 355, 362, 364, 369, 372, 376, 402, 405, 484, 492, 500, 504, 508, 510, 515, 516, 523, 526, 527, 528, 529, 530, 543, 547, 550, 551, 562 Likewise, using a threshold level of <2.0 for non-permissive sites, the following positions corresponding to S. cerevisiae PDC1 residues are relatively non-permissive sites within the multiple sequence alignment: 4, 5, 6, 7, 8, 9, 10, 11, 12, 13, 14, 16, 17, 18, 19, 21, 22, 23, 24, 25, 26, 27, 28, 29, 30, 31, 32, 33, 34, 35, 36, 37, 38, 39, 40, 41, 43, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 106, 107, 109, 110, 111, 112, 113, 114, 115, 116, 117, 118, 119, 120, 121, 122, 124, 125, 127, 128, 129, 130, 131, 132, 133, 134, 135, 136, 137, 139, 140, 141, 142, 143, 145, 146, 148, 149, 150, 151, 152, 153, 155, 156, 157, 159, 160, 161, 162, 163, 164, 165, 167, 168, 169, 170, 171, 172, 175, 176, 179, 182, 183, 193, 196, 197, 199, 200, 201, 203, 204, 206, 207, 208, 211, 212, 213, 214, 215, 216, 217, 218, 219, 220, 221, 222, 224, 225, 226, 227, 231, 233, 234, 235, 236, 237, 238, 240, 241, 242, 243, 244, 245, 246, 247, 248, 249, 250, 251, 252, 253, 254, 256, 257, 258, 259, 260, 261, 262, 263, 264, 265, 267, 268, 269, 270, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 304, 305, 306, 307, 308, 309, 310, 311, 312, 314, 315, 316, 317, 318, 321, 323, 324, 326, 328, 329, 330, 332, 333, 337, 341, 346, 353, 356, 357, 358, 359, 360, 361, 363, 365, 366, 367, 368, 370, 371, 373, 374, 375, 377, 378, 379, 380, 381, 382, 383, 384, 385, 386, 387, 388, 389, 390, 391, 392, 393, 394, 395, 396, 397, 398, 399, 400, 401, 403, 404, 406, 407, 408, 409, 410, 411, 412, 413, 414, 415, 416, 417, 418, 419, 420, 421, 422, 423, 424, 425, 426, 427, 428, 429, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 485, 486, 487, 488, 489, 490, 491, 493, 494, 495, 496, 497, 498, 499, 501, 502, 503, 505, 507, 509, 511, 512, 513, 514, 517, 518, 519, 520, 521, 522, 525, 531, 532, 533, 534, 535, 536, 537, 538, 539, 540, 541, 542, 544, 545, 546, 548, 549, 552, 553, 554, 555, 556, 557, 558, 559, 560, 561.

Accordingly, in some embodiments, the present application provides a nucleic acid molecule encoding a modified decarboxylase, wherein said modified decarboxylase is derived from a corresponding wild-type, unmodified decarboxylase, wherein the sequence of non-permissive sites within said modified decarboxylase is at least about 60%, at least about 70%, at least about 80%, or more preferably at least about 90% identical to the sequence of non-permissive sites within the corresponding wild-type, unmodified decarboxylase. In one embodiment, the threshold level for distinguishing between permissive and non-permissive sites using the code illustrated in FIG. 14 is 1.0. In certain other embodiments, the threshold level for distinguishing between permissive and non-permissive sites using the code illustrated in FIG. 14 is selected from 1.2, 1.4, 1.6, 1.8, and 2.0. In an exemplary embodiment, the modified decarboxylase enzyme is derived from a corresponding wild-type, unmodified decarboxylase selected from SEQ ID NOs: 1-214 and 241-251. In some embodiments, the corresponding wild-type, unmodified decarboxylase is obtained from a yeast microorganism. In a further embodiment, the corresponding wild-type, unmodified decarboxylase is obtained from a yeast microorganism classified into a genera selected from the group consisting of Saccharomyces, Kluyveromyces, Candida, Pichia, Issatchenkia, Debaryomyces, Hansenula, Pachysolen, Yarrowia, Schizosaccharomyces, Tricosporon, Rhodotorula, and Myxozyma. In another further embodiment, the corresponding wild-type, unmodified decarboxylase is obtained from a Saccharomyces yeast. In an exemplary embodiment, the corresponding wild-type, unmodified decarboxylase is obtained from Saccharomyces cerevisiae. In another exemplary embodiment, the corresponding wild-type, unmodified decarboxylase is PDC1 (SEQ ID NO: 241), PDC5 (SEQ ID NO: 242), or PDC6 (SEQ ID NO: 243) of S. cerevisiae.

Another aspect of the application relates to a recombinant microorganism comprising at least one nucleic acid molecule encoding a modified decarboxylase, wherein said decarboxylase has one or more modifications or mutations at positions corresponding to amino acids selected from: (a) aspartic acid 26 of the L. lactis KIVD (SEQ ID NO: 197); (b) histidine 112 of the L. lactis KIVD (SEQ ID NO: 197); (c) histidine 113 of the L. lactis KIVD (SEQ ID NO: 197); (d) glycine 402 of the L. lactis KIVD (SEQ ID NO: 197); and (e) glutamic acid 462 of the L. lactis KIVD (SEQ ID NO: 197). In an exemplary embodiment, the modified decarboxylase enzyme is derived from a corresponding unmodified decarboxylase that is at least about 65%, 70%, 75%, 80%, 85%, 90%, 91%, 92%, 93%, 94%, 95%, 96%, 97%, 98%, 99%, or 99.5% identical to a polypeptide selected from SEQ ID NOs 1-214.

Yet another aspect of the application relates to a recombinant microorganism comprising at least one nucleic acid molecule encoding a modified decarboxylase, wherein said decarboxylase has one or more modifications or mutations at positions corresponding to amino acids selected from: (a) serine 286 of the L. lactis KIVD (SEQ ID NO: 197); (b) glutamine 377 of the L. lactis KIVD (SEQ ID NO: 197); (c) phenylalanine 381 of the L. lactis KIVD (SEQ ID NO: 197); (d) valine 461 of the L. lactis KIVD (SEQ ID NO: 197); (e) isoleucine 465 of the L. lactis KIVD (SEQ ID NO: 197); (f) methionine 538 of the L. lactis KIVD (SEQ ID NO: 197); and (g) phenylalanine 542 of the L. lactis KIVD (SEQ ID NO: 197). In an exemplary embodiment, the modified decarboxylase enzyme is derived from a corresponding unmodified decarboxylase that is at least about 65%, 70%, 75%, 80%, 85%, 90%, 91%, 92%, 93%, 94%, 95%, 96%, 97%, 98%, 99%, or 99.5% identical to a polypeptide selected from SEQ ID NOs 1-214.

Yet another aspect of the application relates to a recombinant microorganism comprising at least one nucleic acid molecule encoding a modified decarboxylase, wherein said decarboxylase has one or more modifications or mutations at positions corresponding to amino acids selected from: (a) phenylalanine 305 of the F. novicida decarboxylase (SEQ ID NO: 198); (b) threonine 397 of the F. novicida decarboxylase (SEQ ID NO: 198); (c) serine 401 of the F. novicida decarboxylase (SEQ ID NO: 198); (d) isoleucine 481 of the F. novicida decarboxylase (SEQ ID NO: 198); (e) leucine 485 of the F. novicida decarboxylase (SEQ ID NO: 198); (f) phenylalanine 556 of the F. novicida decarboxylase (SEQ ID NO: 198); and (g) leucine 560 of the F. novicida decarboxylase (SEQ ID NO: 198). In an exemplary embodiment, the modified decarboxylase enzyme is derived from a corresponding unmodified decarboxylase that is at least about 65%, 70%, 75%, 80%, 85%, 90%, 91%, 92%, 93%, 94%, 95%, 96%, 97%, 98%, 99%, or 99.5% identical to a polypeptide selected from SEQ ID NOs 1-214.

Yet another aspect of the application relates to a recombinant microorganism comprising at least one nucleic acid molecule encoding a modified decarboxylase, wherein said decarboxylase has one or more modifications or mutations at positions corresponding to amino acids selected from: (a) phenylalanine 292 of the S. cerevisiae PDC1 (SEQ ID NO: 241); (b) threonine 388 of the S. cerevisiae PDC1 (SEQ ID NO: 241); (c) alanine 392 of the S. cerevisiae PDC1 (SEQ ID NO: 241); (d) serine 408 of the S. cerevisiae PDC1 (SEQ ID NO: 241); (e) valine 410 of the S. cerevisiae PDC1 (SEQ ID NO: 241); (f) isoleucine 476 of the S. cerevisiae PDC1 (SEQ ID NO: 241); (g) glutamine 552 of the S. cerevisiae PDC1 (SEQ ID NO: 241); and (h) threonine 556 of the S. cerevisiae PDC1 (SEQ ID NO: 241). In an exemplary embodiment, the modified decarboxylase enzyme is derived from a corresponding unmodified decarboxylase that is at least about 65%, 70%, 75%, 80%, 85%, 90%, 91%, 92%, 93%, 94%, 95%, 96%, 97%, 98%, 99%, or 99.5% identical to a wild-type pyruvate decarboxylase. In one embodiment, the wild-type, unmodified pyruvate decarboxylase is obtained from a yeast microorganism. In a further embodiment, the wild-type, unmodified pyruvate decarboxylase is obtained from a yeast microorganism classified into a genera selected from the group consisting of Saccharomyces, Kluyveromyces, Candida, Pichia, Issatchenkia, Debaryomyces, Hansenula, Pachysolen, Yarrowia, Schizosaccharomyces, Tricosporon, Rhodotorula, and Myxozyma. In another further embodiment, the wild-type, unmodified pyruvate decarboxylase is obtained from a Saccharomyces yeast. In an exemplary embodiment, the wild-type, unmodified pyruvate decarboxylase is obtained from Saccharomyces cerevisiae. In another exemplary embodiment, the wild-type, unmodified pyruvate decarboxylase is PDC1 (SEQ ID NO: 241), PDC5 (SEQ ID NO: 242), or PDC6 (SEQ ID NO: 243) of S. cerevisiae. In yet another exemplary embodiment, the wild-type, unmodified pyruvate decarboxylase is selected from SEQ ID NOs: 244-251. In additional embodiments, the recombinant microorganism comprises a deletion or disruption of one or more endogenous pyruvate decarboxylase gene(s). This reduces the cell's ability to produce ethanol, which is particularly desirable in cases in which a higher alcohol such as isobutanol is the desired product. If the host cell contains multiple PDC genes, it is especially preferred to delete or disrupt all of the PDC genes, although it is possible to delete fewer than all such PDC genes. PDC deletion can be accomplished using methods analogous to those described in commonly-owned U.S. Pat. No. 8,017,375.

In accordance with the invention, any number of mutations can be made to the decarboxylase enzymes, and in a preferred aspect, multiple mutations can be made to result in an increased ability to catalyze the conversion of alpha-ketoisovalerate to isobutyraldehyde with high specificity. Such mutations include point mutations, frame shift mutations, deletions, and insertions, with one or more (e.g., one, two, three, four, five, six, seven, eight, nine, ten or more, etc.) point mutations preferred.

### Recombinant Microorganisms Comprising One or More High Performance KIVDs

In addition to isobutanol producing metabolic pathways, a number of biosynthetic pathways use enzymes exhibiting keto-isovalerate decarboxylase (KIVD) activity to catalyze a reaction step, including pathways for the production of isobutanol, 1-propanol, 1-butanol, 2-methyl-1-butanol, 3-methyl-1-butanol, and 2-phenylethanol. A representative list of the engineered biosynthetic pathways utilizing enzymes exhibiting keto-isovalerate decarboxylase (KIVD) activity are described in Table 1.

TABLE 1
Biosynthetic Pathways Utilizing KIVD Activity.

| Biosynthetic Pathway | Reference^{a} |
|---|---|
| Isobutanol | US 2009/0226991 (Feldman et al.), US 2011/0020889 (Feldman et |
| | al.), and US 2010/0143997 (Buelter et al.), Atsumi et al., 2008, |
| | Nature 451: 86-89 |
| 1-Propanol | Atsumi et al., 2008, Nature 451: 86-89 |
| 1-Butanol | Atsumi et al., 2008, Nature 451: 86-89 |
| 2-Methyl-1-butanol | Atsumi et al., 2008, Nature 451: 86-89 |
| 3-Methyl-1-butanol | Atsumi et al., 2008, Nature 451: 86-89 |
| 2-Phenylethanol | Atsumi et al., 2008, Nature 451: 86-89 |

^{a}The contents of each of the references in this table are herein incorporated by reference in their entireties for all purposes.

Each of these biosynthetic pathways comprises a reaction step catalyzed by a 2-keto acid decarboxylase. Specifically, intermediates of the isobutanol, 1-propanol, 1-butanol, 2-methyl-1-butanol, 3-methyl-1-butanol, and 2-phenylethanol pathways are converted to further products by the action of an enzyme exhibiting keto-isovalerate decarboxylase (KIVD) activity—the intermediates are 2-ketoisovalerate, 2-ketobutyrate, 2-ketovalerate, 2-keto-3-methylvalerate, 2-keto-4-methylpentanoate, and phenylpyruvate, respectively. Therefore, the product yield from these biosynthetic pathways will in part depend upon the activity of the enzyme exhibiting keto-isovalerate decarboxylase (KIVD) activity.

As will be understood by one skilled in the art equipped with the present disclosure, the enzymes exhibiting keto-isovalerate decarboxylase (KIVD) activity described herein would have utility in any of the above-described pathways. Thus, in an additional aspect, the present application relates to a recombinant microorganism comprising a biosynthetic pathway requiring an enzyme with keto-isovalerate decarboxylase (KIVD) activity, wherein said recombinant microorganism comprises at least one nucleic acid molecule encoding a polypeptide with keto-isovalerate decarboxylase (KIVD) activity, wherein said polypeptide is at least about 65%, 70%, 75%, 80%, 85%, 90%, 91%, 92%, 93%, 94%, 95%, 96%, 97%, 98%, 99%, or 99.5% identical to a polypeptide selected from SEQ ID NOs 1-214. In a further aspect, the present application relates to a recombinant microorganism comprising a biosynthetic pathway requiring an enzyme with keto-isovalerate decarboxylase (KIVD) activity, wherein said recombinant microorganism comprises at least one nucleic acid molecule encoding a modified decarboxylase, wherein said decarboxylase has one or more modifications or mutations at positions corresponding to amino acids selected from: (a) aspartic acid 26 of the L. lactis KIVD (SEQ ID NO: 197); (b) histidine 112 of the L. lactis KIVD (SEQ ID NO: 197); (c) histidine 113 of the L. lactis KIVD (SEQ ID NO: 197); (d) glycine 402 of the L. lactis KIVD (SEQ ID NO: 197); and (e) glutamic acid 462 of the L. lactis KIVD (SEQ ID NO: 197). In an exemplary embodiment, the modified decarboxylase enzyme is derived from a corresponding unmodified decarboxylase that is at least about 65%, 70%, 75%, 80%, 85%, 90%, 91%, 92%, 93%, 94%, 95%, 96%, 97%, 98%, 99%, or 99.5% identical to a polypeptide selected from SEQ ID NOs 1-214.

In another further aspect, the present application relates to a recombinant microorganism comprising a biosynthetic pathway requiring an enzyme with keto-isovalerate decarboxylase (KIVD) activity, wherein said recombinant microorganism comprises at least one nucleic acid molecule encoding a modified decarboxylase, wherein said decarboxylase has one or more modifications or mutations at positions corresponding to amino acids selected from: (a) serine 286 of the L. lactis KIVD (SEQ ID NO: 197); (b) glutamine 377 of the L. lactis KIVD (SEQ ID NO: 197); (c) phenylalanine 381 of the L. lactis KIVD (SEQ ID NO: 197); (d) valine 461 of the L. lactis KIVD (SEQ ID NO: 197); (e) isoleucine 465 of the L. lactis KIVD (SEQ ID NO: 197); (f) methionine 538 of the L. lactis KIVD (SEQ ID NO: 197); and (g) phenylalanine 542 of the L. lactis KIVD (SEQ ID NO: 197). In an exemplary embodiment, the modified decarboxylase enzyme is derived from a corresponding unmodified decarboxylase that is at least about 65%, 70%, 75%, 80%, 85%, 90%, 91%, 92%, 93%, 94%, 95%, 96%, 97%, 98%, 99%, or 99.5% identical to a polypeptide selected from SEQ ID NOs 1-214.

In yet another further aspect, the present application relates to a recombinant microorganism comprising a biosynthetic pathway requiring an enzyme with keto-isovalerate decarboxylase (KIVD) activity, wherein said recombinant microorganism comprises at least one nucleic acid molecule encoding a modified decarboxylase, wherein said decarboxylase has one or more modifications or mutations at positions corresponding to amino acids selected from: (a) phenylalanine 305 of the F. novicida decarboxylase (SEQ ID NO: 198); (b) threonine 397 of the F. novicida decarboxylase (SEQ ID NO: 198); (c) serine 401 of the F. novicida decarboxylase (SEQ ID NO: 198); (d) isoleucine 481 of the F. novicida decarboxylase (SEQ ID NO: 198); (e) leucine 485 of the F. novicida decarboxylase (SEQ ID NO: 198); (f) phenylalanine 556 of the F. novicida decarboxylase (SEQ ID NO: 198); and (g) leucine 560 of the F. novicida decarboxylase (SEQ ID NO: 198). In an exemplary embodiment, the modified decarboxylase enzyme is derived from a corresponding unmodified decarboxylase that is at least about 65%, 70%, 75%, 80%, 85%, 90%, 91%, 92%, 93%, 94%, 95%, 96%, 97%, 98%, 99%, or 99.5% identical to a polypeptide selected from SEQ ID NOs 1-214.

In yet another further aspect, the present application relates to a recombinant microorganism comprising a biosynthetic pathway requiring an enzyme with keto-isovalerate decarboxylase (KIVD) activity, wherein said recombinant microorganism comprises at least one nucleic acid molecule encoding a modified decarboxylase, wherein said decarboxylase has one or more modifications or mutations at positions corresponding to amino acids selected from: (a) phenylalanine 292 of the S. cerevisiae PDC1 (SEQ ID NO: 241); (b) threonine 388 of the S. cerevisiae PDC1 (SEQ ID NO: 241); (c) alanine 392 of the S. cerevisiae PDC1 (SEQ ID NO: 241); (d) serine 408 of the S. cerevisiae PDC1 (SEQ ID NO: 241); (e) valine 410 of the S. cerevisiae PDC1 (SEQ ID NO: 241); (f) isoleucine 476 of the S. cerevisiae PDC1 (SEQ ID NO: 241); (g) glutamine 552 of the S. cerevisiae PDC1 (SEQ ID NO: 241); and (h) threonine 556 of the S. cerevisiae PDC1 (SEQ ID NO: 241). In an exemplary embodiment, the modified decarboxylase enzyme is derived from a corresponding unmodified decarboxylase that is at least about 65%, 70%, 75%, 80%, 85%, 90%, 91%, 92%, 93%, 94%, 95%, 96%, 97%, 98%, 99%, or 99.5% identical to a wild-type pyruvate decarboxylase. In one embodiment, the wild-type, unmodified pyruvate decarboxylase is obtained from a yeast microorganism. In a further embodiment, the wild-type, unmodified pyruvate decarboxylase is obtained from a yeast microorganism classified into a genera selected from the group consisting of Saccharomyces, Kluyveromyces, Candida, Pichia, Issatchenkia, Debaryomyces, Hansenula, Pachysolen, Yarrowia, Schizosaccharomyces, Tricosporon, Rhodotorula, and Myxozyma. In another further embodiment, the wild-type, unmodified pyruvate decarboxylase is obtained from a Saccharomyces yeast. In an exemplary embodiment, the wild-type, unmodified pyruvate decarboxylase is obtained from Saccharomyces cerevisiae. In another exemplary embodiment, the wild-type, unmodified pyruvate decarboxylase is PDC1 (SEQ ID NO: 241), PDC5 (SEQ ID NO: 242), or PDC6 (SEQ ID NO: 243) of S. cerevisiae. In yet another exemplary embodiment, the wild-type, unmodified pyruvate decarboxylase is selected from SEQ ID NOs: 244-251. In additional embodiments, the recombinant microorganism comprises a deletion or disruption of one or more endogenous pyruvate decarboxylase gene(s).

As used herein, a biosynthetic pathway requiring an enzyme with keto-isovalerate decarboxylase (KIVD) activity refers to any metabolic pathway which utilizes an enzyme with keto-isovalerate decarboxylase (KIVD) activity to convert a substrate to product conversion, e.g., starting with substrates such as 2-ketoisovalerate, 2-ketobutyrate, 2-ketovalerate, 2-keto-3-methylvalerate, 2-keto-4-methylpentanoate, and phenylpyruvate. Examples of biosynthetic pathway requiring an enzyme with keto-isovalerate decarboxylase (KIVD) activity include, but are not limited to, isobutanol, 1-propanol, 1-butanol, 2-methyl-1-butanl, 3-methyl-1-butanol, and 2-phenylethanol metabolic pathways. In an exemplary embodiment, the biosynthetic pathway requiring an enzyme with keto-isovalerate decarboxylase (KIVD) activity is an isobutanol-producing metabolic pathway. The metabolic pathway may naturally occur in a microorganism or arise from the introduction of one or more heterologous polynucleotides through genetic engineering. In an exemplary embodiment, the recombinant microorganisms expressing the biosynthetic pathway requiring an enzyme with keto-isovalerate decarboxylase (KIVD) activity are yeast cells.

### The Microorganism in General

As described herein, the recombinant microorganisms of the present invention can express a plurality of heterologous and/or native enzymes involved in pathways for the production of a beneficial metabolite such as isobutanol.

As described herein, “engineered” or “modified” microorganisms are produced via the introduction of genetic material into a host or parental microorganism of choice and/or by modification of the expression of native genes, thereby modifying or altering the cellular physiology and biochemistry of the microorganism. Through the introduction of genetic material and/or the modification of the expression of native genes the parental microorganism acquires new properties, e.g., the ability to produce a new, or greater quantities of, an intracellular and/or extracellular metabolite. As described herein, the introduction of genetic material into and/or the modification of the expression of native genes in a parental microorganism results in a new or modified ability to produce beneficial metabolites such as isobutanol from a suitable carbon source. The genetic material introduced into and/or the genes modified for expression in the parental microorganism contains gene(s), or parts of genes, coding for one or more of the enzymes involved in a biosynthetic pathway for the production of isobutanol and may also include additional elements for the expression and/or regulation of expression of these genes, e.g., promoter sequences.

In addition to the introduction of a genetic material into a host or parental microorganism, an engineered or modified microorganism can also include the alteration, disruption, deletion or knocking-out of a gene or polynucleotide to alter the cellular physiology and biochemistry of the microorganism. Through the alteration, disruption, deletion or knocking-out of a gene or polynucleotide, the microorganism acquires new or improved properties (e.g., the ability to produce a new metabolite or greater quantities of an intracellular metabolite, to improve the flux of a metabolite down a desired pathway, and/or to reduce the production of by-products).

Recombinant microorganisms provided herein may also produce metabolites in quantities not available in the parental microorganism. A “metabolite” refers to any substance produced by metabolism or a substance necessary for or taking part in a particular metabolic process. A metabolite can be an organic compound that is a starting material (e.g., glucose or pyruvate), an intermediate (e.g., 2-ketoisovalerate), or an end product (e.g., isobutanol) of metabolism. Metabolites can be used to construct more complex molecules, or they can be broken down into simpler ones. Intermediate metabolites may be synthesized from other metabolites, perhaps used to make more complex substances, or broken down into simpler compounds, often with the release of chemical energy.

The disclosure identifies specific genes useful in the methods, compositions and organisms of the disclosure; however it will be recognized that absolute identity to such genes is not necessary. For example, changes in a particular gene or polynucleotide comprising a sequence encoding a polypeptide or enzyme can be performed and screened for activity. Typically such changes comprise conservative mutations and silent mutations. Such modified or mutated polynucleotides and polypeptides can be screened for expression of a functional enzyme using methods known in the art.

Due to the inherent degeneracy of the genetic code, other polynucleotides which encode substantially the same or functionally equivalent polypeptides can also be used to clone and express the polynucleotides encoding such enzymes.

As will be understood by those of skill in the art, it can be advantageous to modify a coding sequence to enhance its expression in a particular host. The genetic code is redundant with 64 possible codons, but most organisms typically use a subset of these codons. The codons that are utilized most often in a species are called optimal codons, and those not utilized very often are classified as rare or low-usage codons. Codons can be substituted to reflect the preferred codon usage of the host, in a process sometimes called “codon optimization” or “controlling for species codon bias.”

Optimized coding sequences containing codons preferred by a particular prokaryotic or eukaryotic host (Murray et al., 1989, Nucl Acids Res. 17: 477-508) can be prepared, for example, to increase the rate of translation or to produce recombinant RNA transcripts having desirable properties, such as a longer half-life, as compared with transcripts produced from a non-optimized sequence. Translation stop codons can also be modified to reflect host preference. For example, typical stop codons for S. cerevisiae and mammals are UAA and UGA, respectively. The typical stop codon for monocotyledonous plants is UGA, whereas insects and E. coli commonly use UAA as the stop codon (Dalphin et al., 1996, Nucl Acids Res. 24: 216-8).

Those of skill in the art will recognize that, due to the degenerate nature of the genetic code, a variety of DNA compounds differing in their nucleotide sequences can be used to encode a given enzyme of the disclosure. The native DNA sequence encoding the biosynthetic enzymes described above are referenced herein merely to illustrate an embodiment of the disclosure, and the disclosure includes DNA compounds of any sequence that encode the amino acid sequences of the polypeptides and proteins of the enzymes utilized in the methods of the disclosure. In similar fashion, a polypeptide can typically tolerate one or more amino acid substitutions, deletions, and insertions in its amino acid sequence without loss or significant loss of a desired activity. The disclosure includes such polypeptides with different amino acid sequences than the specific proteins described herein so long as the modified or variant polypeptides have the enzymatic anabolic or catabolic activity of the reference polypeptide. Furthermore, the amino acid sequences encoded by the DNA sequences shown herein merely illustrate embodiments of the disclosure.

In addition, homologs of enzymes useful for generating metabolites are encompassed by the microorganisms and methods provided herein.

As used herein, two proteins (or a region of the proteins) are substantially homologous when the amino acid sequences have at least about 30%, 40%, 50% 60%, 65%, 70%, 75%, 80%, 85%, 90%, 91%, 92%, 93%, 94%, 95%, 96%, 97%, 98%, or 99% identity. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In one embodiment, the length of a reference sequence aligned for comparison purposes is at least 30%, typically at least 40%, more typically at least 50%, even more typically at least 60%, and even more typically at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art (See, e.g., Pearson W. R., 1994, Methods in Mol Biol 25: 365-89).

The following six groups each contain amino acids that are conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Alanine (A), Valine (V), and 6) Phenylalanine (F), Tyrosine (Y), Tryptophan (W).

Sequence homology for polypeptides, which is also referred to as percent sequence identity, is typically measured using sequence analysis software. See commonly owned and co-pending application US 2009/0226991. A typical algorithm used comparing a molecule sequence to a database containing a large number of sequences from different organisms is the computer program BLAST. When searching a database containing sequences from a large number of different organisms, it is typical to compare amino acid sequences. Database searching using amino acid sequences can be measured by algorithms described in commonly owned U.S. Pat. No. 8,017,375.

It is understood that a range of microorganisms can be modified to include an isobutanol producing metabolic pathway suitable for the production of isobutanol. In various embodiments, the microorganisms may be selected from yeast microorganisms. Yeast microorganisms for the production of isobutanol may be selected based on certain characteristics:

One characteristic may include the property that the microorganism is selected to convert various carbon sources into isobutanol. The term “carbon source” generally refers to a substance suitable to be used as a source of carbon for prokaryotic or eukaryotic cell growth. Examples of suitable carbon sources are described in commonly owned U.S. Pat. No. 8,017,375. Accordingly, in one embodiment, the recombinant microorganism herein disclosed can convert a variety of carbon sources to products, including but not limited to glucose, galactose, mannose, xylose, arabinose, lactose, sucrose, CO₂, and mixtures thereof.

The recombinant microorganism may thus further include a pathway for the production of isobutanol from five-carbon (pentose) sugars including xylose. Most yeast species metabolize xylose via a complex route, in which xylose is first reduced to xylitol via a xylose reductase (XR) enzyme. The xylitol is then oxidized to xylulose via a xylitol dehydrogenase (XDH) enzyme. The xylulose is then phosphorylated via a xylulokinase (XK) enzyme. This pathway operates inefficiently in yeast species because it introduces a redox imbalance in the cell. The xylose-to-xylitol step uses primarily NADPH as a cofactor (generating NADP+), whereas the xylitol-to-xylulose step uses NAD+ as a cofactor (generating NADH). Other processes must operate to restore the redox imbalance within the cell. This often means that the organism cannot grow anaerobically on xylose or other pentose sugars. Accordingly, a yeast species that can efficiently ferment xylose and other pentose sugars into a desired fermentation product is therefore very desirable.

Thus, in one aspect, the recombinant microorganism is engineered to express a functional exogenous xylose isomerase. Exogenous xylose isomerases (XI) functional in yeast are known in the art. See, e.g., Rajgarhia et al., U.S. Pat. No. 7,943,366, which is herein incorporated by reference in its entirety. In an embodiment according to this aspect, the exogenous XI gene is operatively linked to promoter and terminator sequences that are functional in the yeast cell. In a preferred embodiment, the recombinant microorganism further has a deletion or disruption of a native gene that encodes for an enzyme (e.g., XR and/or XDH) that catalyzes the conversion of xylose to xylitol. In a further preferred embodiment, the recombinant microorganism also contains a functional, exogenous xylulokinase (XK) gene operatively linked to promoter and terminator sequences that are functional in the yeast cell. In one embodiment, the xylulokinase (XK) gene is overexpressed.

In one embodiment, the yeast microorganism has reduced or no pyruvate decarboxylase (PDC) activity. PDC catalyzes the decarboxylation of pyruvate to acetaldehyde, which is then reduced to ethanol by ADH via an oxidation of NADH to NAD+. Ethanol production is the main pathway to oxidize the NADH from glycolysis. Deletion, disruption, or mutation of this pathway increases the pyruvate and the reducing equivalents (NADH) available for a biosynthetic pathway which uses pyruvate as the starting material and/or as an intermediate. Accordingly, deletion, disruption, or mutation of one or more genes encoding for pyruvate decarboxylase and/or a positive transcriptional regulator thereof can further increase the yield of the desired pyruvate-derived metabolite (e.g., isobutanol). In one embodiment, said pyruvate decarboxylase gene targeted for disruption, deletion, or mutation is selected from the group consisting of PDC1, PDC5, and PDC6, or homologs or variants thereof. In another embodiment, all three of PDC1, PDC5, and PDC6 are targeted for disruption, deletion, or mutation. In yet another embodiment, a positive transcriptional regulator of the PDC1, PDC5, and/or PDC6 is targeted for disruption, deletion or mutation. In one embodiment, said positive transcriptional regulator is PDC2, or homologs or variants thereof.

As is understood by those skilled in the art, there are several additional mechanisms available for reducing or disrupting the activity of a protein encoded by PDC1, PDC5, PDC6, and/or PDC2, including, but not limited to, the use of a regulated promoter, use of a weak constitutive promoter, disruption of one of the two copies of the gene in a diploid yeast, disruption of both copies of the gene in a diploid yeast, expression of an anti-sense nucleic acid, expression of an siRNA, over expression of a negative regulator of the endogenous promoter, alteration of the activity of an endogenous or heterologous gene, use of a heterologous gene with lower specific activity, the like or combinations thereof. Yeast strains with reduced PDC activity are described in commonly owned U.S. Pat. No. 8,017,375, as well as commonly owned and co-pending US Patent Publication No. 2011/0183392.

In another embodiment, the microorganism has reduced glycerol-3-phosphate dehydrogenase (GPD) activity. GPD catalyzes the reduction of dihydroxyacetone phosphate (DHAP) to glycerol-3-phosphate (G3P) via the oxidation of NADH to NAD+. Glycerol is then produced from G3P by Glycerol-3-phosphatase (GPP). Glycerol production is a secondary pathway to oxidize excess NADH from glycolysis. Reduction or elimination of this pathway would increase the pyruvate and reducing equivalents (NADH) available for the production of a pyruvate-derived metabolite (e.g., isobutanol). Thus, disruption, deletion, or mutation of the genes encoding for glycerol-3-phosphate dehydrogenases can further increase the yield of the desired metabolite (e.g., isobutanol). Yeast strains with reduced GPD activity are described in commonly owned and co-pending US Patent Publication Nos. 2011/0020889 and 2011/0183392.

In yet another embodiment, the microorganism has reduced 3-keto acid reductase (3-KAR) activity. 3-KARs catalyze the conversion of 3-keto acids (e.g., acetolactate) to 3-hydroxyacids (e.g., DH2 MB). Yeast strains with reduced 3-KAR activity are described in commonly owned U.S. Pat. Nos. 8,133,715, 8,153,415, and 8,158,404, which are herein incorporated by reference in their entireties.

In yet another embodiment, the microorganism has reduced aldehyde dehydrogenase (ALDH) activity. Aldehyde dehydrogenases catalyze the conversion of aldehydes (e.g., isobutyraldehyde) to acid by-products (e.g., isobutyrate). Yeast strains with reduced ALDH activity are described in commonly owned U.S. Pat. Nos. 8,133,715, 8,153,415, and 8,158,404, which are herein incorporated by reference in their entireties.

In one embodiment, the yeast microorganisms may be selected from the “Saccharomyces Yeast Clade”, as described in commonly owned U.S. Pat. No. 8,017,375.

The term “Saccharomyces sensu stricto” taxonomy group is a cluster of yeast species that are highly related to S. cerevisiae (Rainieri et al., 2003, J. Biosci Bioengin 96: 1-9). Saccharomyces sensu stricto yeast species include but are not limited to S. cerevisiae, S. kudriavzevii, S. mikatae, S. bayanus, S. uvarum, S. carocanis and hybrids derived from these species (Masneuf et al., 1998, Yeast 7: 61-72).

An ancient whole genome duplication (WGD) event occurred during the evolution of the hemiascomycete yeast and was discovered using comparative genomic tools (Kellis et al., 2004, Nature 428: 617-24; Dujon et al., 2004, Nature 430:35-44; Langkjaer et al., 2003, Nature 428: 848-52; Wolfe et al., 1997, Nature 387: 708-13). Using this major evolutionary event, yeast can be divided into species that diverged from a common ancestor following the WGD event (termed “post-WGD yeast” herein) and species that diverged from the yeast lineage prior to the WGD event (termed “pre-WGD yeast” herein).

Accordingly, in one embodiment, the yeast microorganism may be selected from a post-WGD yeast genus, including but not limited to Saccharomyces and Candida. The favored post-WGD yeast species include: S. cerevisiae, S. uvarum, S. bayanus, S. paradoxus, S. castelli, and C. glabrata.

In another embodiment, the yeast microorganism may be selected from a pre-whole genome duplication (pre-WGD) yeast genus including but not limited to Saccharomyces, Kluyveromyces, Candida, Pichia, Issatchenkia, Debaryomyces, Hansenula, Yarrowia and, Schizosaccharomyces. Representative pre-WGD yeast species include: S. kluyveri, K. thermotolerans, K. marxianus, K. waltii, K. lactis, C. tropicalis, P. pastoris, P. anomala, P. stipitis, I. orientalis, I. occidentalis, I. scutulata, D. hansenii, H. anomala, Y. lipolytica, and S. pombe.

A yeast microorganism may be either Crabtree-negative or Crabtree-positive as described in described in commonly owned U.S. Pat. No. 8,017,375. In one embodiment the yeast microorganism may be selected from yeast with a Crabtree-negative phenotype including but not limited to the following genera: Saccharomyces, Kluyveromyces, Pichia, Issatchenkia, Hansenula, and Candida. Crabtree-negative species include but are not limited to: S. kluyveri, K. lactis, K. marxianus, P. anomala, P. stipitis, I. orientalis, I. occidentalis, I. scutulata, H. anomala, and C. utills. In another embodiment, the yeast microorganism may be selected from yeast with a Crabtree-positive phenotype, including but not limited to Saccharomyces, Kluyveromyces, Zygosaccharomyces, Debaryomyces, Pichia and Schizosaccharomyces. Crabtree-positive yeast species include but are not limited to: S. cerevisiae, S. uvarum, S. bayanus, S. paradoxus, S. castelli, K. thermotolerans, C. glabrata, Z. bailli, Z. rouxii, D. hansenii, P. pastorius, and S. pombe.

Another characteristic may include the property that the microorganism is that it is non-fermenting. In other words, it cannot metabolize a carbon source anaerobically while the yeast is able to metabolize a carbon source in the presence of oxygen. Nonfermenting yeast refers to both naturally occurring yeasts as well as genetically modified yeast. During anaerobic fermentation with fermentative yeast, the main pathway to oxidize the NADH from glycolysis is through the production of ethanol. Ethanol is produced by alcohol dehydrogenase (ADH) via the reduction of acetaldehyde, which is generated from pyruvate by pyruvate decarboxylase (PDC). In one embodiment, a fermentative yeast can be engineered to be non-fermentative by the reduction or elimination of the native PDC activity. Thus, most of the pyruvate produced by glycolysis is not consumed by PDC and is available for the isobutanol pathway. Deletion of this pathway increases the pyruvate and the reducing equivalents available for the biosynthetic pathway. Fermentative pathways contribute to low yield and low productivity of pyruvate-derived metabolites such as isobutanol. Accordingly, deletion of one or more PDC genes may increase yield and productivity of a desired metabolite (e.g., isobutanol).

In some embodiments, the recombinant microorganisms may be microorganisms that are non-fermenting yeast microorganisms, including, but not limited to those, classified into a genera selected from the group consisting of Tricosporon, Rhodotorula, Myxozyma, or Candida. In a specific embodiment, the non-fermenting yeast is C. xestobii.

Yeast microorganisms within the scope of the invention may have reduced enzymatic activity such as reduced 3-KAR, ALDH, PDC, or GPD activity. The term “reduced” as used herein with respect to a particular polypeptide activity refers to a lower level of polypeptide activity than that measured in a comparable yeast cell of the same species. The term reduced also refers to the elimination of polypeptide activity as compared to a comparable yeast cell of the same species. Thus, yeast cells lacking activity for an endogenous 3-KAR, ALDH, PDC, or GPD are considered to have reduced activity for 3-KAR, ALDH, PDC, or GPD since most, if not all, comparable yeast strains have at least some activity for 3-KAR, ALDH, PDC, or GPD. Such reduced 3-KAR, ALDH, PDC, or GPD activities can be the result of lower 3-KAR, ALDH, PDC, or GPD concentration (e.g., via reduced expression), lower specific activity of the 3-KAR, ALDH, PDC, or GPD, or a combination thereof. Many different methods can be used to make yeast having reduced 3-KAR, ALDH, PDC, or GPD activity. For example, a yeast cell can be engineered to have a disrupted 3-KAR-, ALDH-, PDC-, or GPD-encoding locus using common mutagenesis or knock-out technology. See, e.g., Methods in Yeast Genetics (1997 edition), Adams, Gottschling, Kaiser, and Stems, Cold Spring Harbor Press (1998). In addition, a yeast cell can be engineered to partially or completely remove the coding sequence for a particular 3-KAR, ALDH, PDC, or GPD. Furthermore, the promoter sequence and/or associated regulatory elements can be mutated, disrupted, or deleted to reduce the expression of a 3-KAR, ALDH, PDC, or GPD. Moreover, certain point-mutation(s) can be introduced which results in a 3-KAR, ALDH, PDC, or GPD with reduced activity. Also included within the scope of this invention are yeast strains which when found in nature, are substantially free of one or more 3-KAR, ALDH, PDC, or GPD activities.

Alternatively, antisense technology can be used to reduce 3-KAR, ALDH, PDC, or GPD activity. For example, yeasts can be engineered to contain a cDNA that encodes an antisense molecule that prevents a 3-KAR, ALDH, PDC, or GPD from being made. The term “antisense molecule” as used herein encompasses any nucleic acid molecule that contains sequences that correspond to the coding strand of an endogenous polypeptide. An antisense molecule also can have flanking sequences (e.g., regulatory sequences). Thus antisense molecules can be ribozymes or antisense oligonucleotides. A ribozyme can have any general structure including, without limitation, hairpin, hammerhead, or axhead structures, provided the molecule cleaves RNA.

In alternative embodiments, the recombinant microorganisms may be derived from bacterial microorganisms. In various embodiments the recombinant microorganism may be selected from a genus of Citrobacter, Corynebacterium, Lactobacillus, Lactococcus, Salmonella, Enterobacter, Enterococcus, Erwinia, Pantoea, Morganella, Pectobacterium, Proteus, Serratia, Shigella, and Klebsiella. In one specific embodiment, the recombinant microorganism is a lactic acid bacteria such as, for example, a microorganism derived from the Lactobacillus or Lactococcus genus.

### General Methods

Methods for the identification of homologous enzymes exhibiting KIVD activity, as well as methods for gene insertion, gene deletion, and gene overexpression may be found in commonly-owned U.S. Pat. Nos. 8,017,375, 8,017,376, 8,071,358, 8,097,440, 8,133,175, 8,153,415, 8,158,404, and 8,232,089, each of which is herein incorporated by reference in its entirety for all purposes.

### Methods of Using Recombinant Microorganisms for Isobutanol Production

In one aspect, the present application provides methods of producing a desired metabolite using a recombinant described herein. In one embodiment, the recombinant microorganism comprises a biosynthetic pathway requiring an enzyme with keto-isovalerate decarboxylase (KIVD) activity, wherein said recombinant microorganism comprises at least one nucleic acid molecule encoding a polypeptide with keto-isovalerate decarboxylase (KIVD) activity, wherein said polypeptide is at least about 65%, 70%, 75%, 80%, 85%, 90%, 91%, 92%, 93%, 94%, 95%, 96%, 97%, 98%, 99%, or 99.5% identical to a polypeptide selected from SEQ ID NOs 1-214. In another embodiment, the recombinant microorganism comprises a biosynthetic pathway requiring an enzyme with keto-isovalerate decarboxylase (KIVD) activity, wherein said recombinant microorganism comprises at least one nucleic acid molecule encoding a modified decarboxylase, wherein said decarboxylase has one or more modifications or mutations at positions corresponding to amino acids selected from: (a) aspartic acid 26 of the L. lactis KIVD (SEQ ID NO: 197); (b) histidine 112 of the L. lactis KIVD (SEQ ID NO: 197); (c) histidine 113 of the L. lactis KIVD (SEQ ID NO: 197); (d) glycine 402 of the L. lactis KIVD (SEQ ID NO: 197); and (e) glutamic acid 462 of the L. lactis KIVD (SEQ ID NO: 197). In an exemplary embodiment, the modified decarboxylase enzyme is derived from a corresponding unmodified decarboxylase that is at least about 65%, 70%, 75%, 80%, 85%, 90%, 91%, 92%, 93%, 94%, 95%, 96%, 97%, 98%, 99%, or 99.5% identical to a polypeptide selected from SEQ ID NOs 1-214. In yet another embodiment, the recombinant microorganism comprises a biosynthetic pathway requiring an enzyme with keto-isovalerate decarboxylase (KIVD) activity, wherein said recombinant microorganism comprises at least one nucleic acid molecule encoding a modified decarboxylase, wherein said decarboxylase has one or more modifications or mutations at positions corresponding to amino acids selected from: (a) serine 286 of the L. lactis KIVD (SEQ ID NO: 197); (b) glutamine 377 of the L. lactis KIVD (SEQ ID NO: 197); (c) phenylalanine 381 of the L. lactis KIVD (SEQ ID NO: 197); (d) valine 461 of the L. lactis KIVD (SEQ ID NO: 197); (e) isoleucine 465 of the L. lactis KIVD (SEQ ID NO: 197); (f) methionine 538 of the L. lactis KIVD (SEQ ID NO: 197); and (g) phenylalanine 542 of the L. lactis KIVD (SEQ ID NO: 197). In an exemplary embodiment, the modified decarboxylase enzyme is derived from a corresponding unmodified decarboxylase that is at least about 65%, 70%, 75%, 80%, 85%, 90%, 91%, 92%, 93%, 94%, 95%, 96%, 97%, 98%, 99%, or 99.5% identical to a polypeptide selected from SEQ ID NOs 1-214. In yet another embodiment, the recombinant microorganism comprises a biosynthetic pathway requiring an enzyme with keto-isovalerate decarboxylase (KIVD) activity, wherein said recombinant microorganism comprises at least one nucleic acid molecule encoding a modified decarboxylase, wherein said decarboxylase has one or more modifications or mutations at positions corresponding to amino acids selected from: (a) phenylalanine 305 of the F. novicida decarboxylase (SEQ ID NO: 198); (b) threonine 397 of the F. novicida decarboxylase (SEQ ID NO: 198); (c) serine 401 of the F. novicida decarboxylase (SEQ ID NO: 198); (d) isoleucine 481 of the F. novicida decarboxylase (SEQ ID NO: 198); (e) leucine 485 of the F. novicida decarboxylase (SEQ ID NO: 198); (f) phenylalanine 556 of the F. novicida decarboxylase (SEQ ID NO: 198); and (g) leucine 560 of the F. novicida decarboxylase (SEQ ID NO: 198). In an exemplary embodiment, the modified decarboxylase enzyme is derived from a corresponding unmodified decarboxylase that is at least about 65%, 70%, 75%, 80%, 85%, 90%, 91%, 92%, 93%, 94%, 95%, 96%, 97%, 98%, 99%, or 99.5% A identical to a polypeptide selected from SEQ ID NOs 1-214. In yet another embodiment, the recombinant microorganism comprises a biosynthetic pathway requiring an enzyme with keto-isovalerate decarboxylase (KIVD) activity, wherein said recombinant microorganism comprises at least one nucleic acid molecule encoding a modified decarboxylase, wherein said decarboxylase has one or more modifications or mutations at positions corresponding to amino acids selected from: (a) phenylalanine 292 of the S. cerevisiae PDC1 (SEQ ID NO: 241); (b) threonine 388 of the S. cerevisiae PDC1 (SEQ ID NO: 241); (c) alanine 392 of the S. cerevisiae PDC1 (SEQ ID NO: 241); (d) serine 408 of the S. cerevisiae PDC1 (SEQ ID NO: 241); (e) valine 410 of the S. cerevisiae PDC1 (SEQ ID NO: 241); (f) isoleucine 476 of the S. cerevisiae PDC1 (SEQ ID NO: 241); (g) glutamine 552 of the S. cerevisiae PDC1 (SEQ ID NO: 241); and (h) threonine 556 of the S. cerevisiae PDC1 (SEQ ID NO: 241). In an exemplary embodiment, the modified decarboxylase enzyme is derived from a corresponding unmodified decarboxylase that is at least about 65%, 70%, 75%, 80%, 85%, 90%, 91%, 92%, 93%, 94%, 95%, 96%, 97%, 98%, 99%, or 99.5% identical to a wild-type pyruvate decarboxylase. In one embodiment, the wild-type, unmodified pyruvate decarboxylase is obtained from a yeast microorganism. In a further embodiment, the wild-type, unmodified pyruvate decarboxylase is obtained from a yeast microorganism classified into a genera selected from the group consisting of Saccharomyces, Kluyveromyces, Candida, Pichia, Issatchenkia, Debaryomyces, Hansenula, Pachysolen, Yarrowia, Schizosaccharomyces, Tricosporon, Rhodotorula, and Myxozyma. In another further embodiment, the wild-type, unmodified pyruvate decarboxylase is obtained from a Saccharomyces yeast. In an exemplary embodiment, the wild-type, unmodified pyruvate decarboxylase is obtained from Saccharomyces cerevisiae. In another exemplary embodiment, the wild-type, unmodified pyruvate decarboxylase is PDC1 (SEQ ID NO: 241), PDC5 (SEQ ID NO: 242), or PDC6 (SEQ ID NO: 243) of S. cerevisiae. In yet another exemplary embodiment, the wild-type, unmodified pyruvate decarboxylase is selected from SEQ ID NOs: 244-251. In additional embodiments, the recombinant microorganism comprises a deletion or disruption of one or more endogenous pyruvate decarboxylase gene(s).

In an exemplary embodiment, the biosynthetic pathway is a pathway for the production of a beneficial metabolite selected from isobutanol, 1-propanol, 1-butanol, 2-methyl-1-butanl, 3-methyl-1-butanol, and 2-phenylethanol. In a further exemplary embodiment, the beneficial metabolite is isobutanol.

In a method to produce a beneficial metabolite (e.g., isobutanol) from a carbon source, the recombinant microorganism is cultured in an appropriate culture medium containing a carbon source. In certain embodiments, the method further includes isolating the beneficial metabolite (e.g., isobutanol) from the culture medium. For example, a beneficial metabolite (e.g., isobutanol) may be isolated from the culture medium by any method known to those skilled in the art, such as distillation, pervaporation, or liquid-liquid extraction. In certain exemplary embodiments, the beneficial metabolite is selected from isobutanol, 1-propanol, 1-butanol, 2-methyl-1-butanol, 3-methyl-1-butanol, and 2-phenylethanol. In a further exemplary embodiment, the beneficial metabolite is isobutanol.

In one embodiment, the recombinant microorganism may produce the beneficial metabolite (e.g., isobutanol) from a carbon source at a yield of at least 5 percent theoretical. In another embodiment, the microorganism may produce the beneficial metabolite (e.g., isobutanol) from a carbon source at a yield of at least about 10 percent, at least about 15 percent, about least about 20 percent, at least about 25 percent, at least about 30 percent, at least about 35 percent, at least about 40 percent, at least about 45 percent, at least about 50 percent, at least about 55 percent, at least about 60 percent, at least about 65 percent, at least about 70 percent, at least about 75 percent, at least about 80 percent, at least about 85 percent, at least about 90 percent, at least about 95 percent, or at least about 97.5 percent theoretical. In a specific embodiment, the beneficial metabolite is isobutanol.

### Distillers Dried Grains Comprising Spent Yeast Biocatalysts

In an economic fermentation process, as many of the products of the fermentation as possible, including the co-products that contain biocatalyst cell material, should have value. Insoluble material produced during fermentations using grain feedstocks, like corn, is frequently sold as protein and vitamin rich animal feed called distillers dried grains (DDG). See, e.g., commonly owned and co-pending U.S. Publication No. 2009/0215137, which is herein incorporated by reference in its entirety for all purposes. As used herein, the term “DDG” generally refers to the solids remaining after a fermentation, usually consisting of unconsumed feedstock solids, remaining nutrients, protein, fiber, and oil, as well as spent yeast biocatalysts or cell debris therefrom that are recovered by further processing from the fermentation, usually by a solids separation step such as centrifugation.

Distillers dried grains may also include soluble residual material from the fermentation, or syrup, and are then referred to as “distillers dried grains and solubles” (DDGS). Use of DDG or DDGS as animal feed is an economical use of the spent biocatalyst following an industrial scale fermentation process.

Accordingly, in one aspect, the present invention provides an animal feed product comprised of DDG derived from a fermentation process for the production of a beneficial metabolite (e.g., isobutanol), wherein said DDG comprise a spent yeast biocatalyst of the present invention. In an exemplary embodiment, said spent yeast biocatalyst has been engineered to comprise at least one nucleic acid molecule encoding a polypeptide with keto-isovalerate decarboxylase (KIVD) activity, wherein said polypeptide is at least about 65%, 70%, 75%, 80%, 85%, 90%, 91%, 92%, 93%, 94%, 95%, 96%, 97%, 98%, 99%, or 99.5% A identical to a polypeptide selected from SEQ ID NOs 1-214. In another exemplary embodiment, said spent yeast biocatalyst has been engineered to comprise at least one nucleic acid molecule encoding a polypeptide with keto-isovalerate decarboxylase (KIVD) activity, wherein said spent yeast biocatalyst comprises at least one nucleic acid molecule encoding a modified decarboxylase, wherein said decarboxylase has one or more modifications or mutations at positions corresponding to amino acids selected from: (a) aspartic acid 26 of the L. lactis KIVD (SEQ ID NO: 197); (b) histidine 112 of the L. lactis KIVD (SEQ ID NO: 197); (c) histidine 113 of the L. lactis KIVD (SEQ ID NO: 197); (d) glycine 402 of the L. lactis KIVD (SEQ ID NO: 197); and (e) glutamic acid 462 of the L. lactis KIVD (SEQ ID NO: 197). In an exemplary embodiment, the modified decarboxylase enzyme is derived from a corresponding unmodified decarboxylase that is at least about 65%, 70%, 75%, 80%, 85%, 90%, 91%, 92%, 93%, 94%, 95%, 96%, 97%, 98%, 99%, or 99.5% identical to a polypeptide selected from SEQ ID NOs 1-214. In yet another exemplary embodiment, said spent yeast biocatalyst has been engineered to comprise at least one nucleic acid molecule encoding a polypeptide with keto-isovalerate decarboxylase (KIVD) activity, wherein said spent yeast biocatalyst comprises at least one nucleic acid molecule encoding a modified decarboxylase, wherein said decarboxylase has one or more modifications or mutations at positions corresponding to amino acids selected from: (a) serine 286 of the L. lactis KIVD (SEQ ID NO: 197); (b) glutamine 377 of the L. lactis KIVD (SEQ ID NO: 197); (c) phenylalanine 381 of the L. lactis KIVD (SEQ ID NO: 197); (d) valine 461 of the L. lactis KIVD (SEQ ID NO: 197); (e) isoleucine 465 of the L. lactis KIVD (SEQ ID NO: 197); (f) methionine 538 of the L. lactis KIVD (SEQ ID NO: 197); and (g) phenylalanine 542 of the L. lactis KIVD (SEQ ID NO: 197). In an exemplary embodiment, the modified decarboxylase enzyme is derived from a corresponding unmodified decarboxylase that is at least about 65%, 70%, 75%, 80%, 85%, 90%, 91%, 92%, 93%, 94%, 95%, 96%, 97%, 98%, 99%, or 99.5% identical to a polypeptide selected from SEQ ID NOs 1-214. In yet another exemplary embodiment, said spent yeast biocatalyst has been engineered to comprise at least one nucleic acid molecule encoding a polypeptide with keto-isovalerate decarboxylase (KIVD) activity, wherein said spent yeast biocatalyst comprises at least one nucleic acid molecule encoding a modified decarboxylase, wherein said decarboxylase has one or more modifications or mutations at positions corresponding to amino acids selected from: (a) phenylalanine 305 of the F. novicida decarboxylase (SEQ ID NO: 198); (b) threonine 397 of the F. novicida decarboxylase (SEQ ID NO: 198); (c) serine 401 of the F. novicida decarboxylase (SEQ ID NO: 198); (d) isoleucine 481 of the F. novicida decarboxylase (SEQ ID NO: 198); (e) leucine 485 of the F. novicida decarboxylase (SEQ ID NO: 198); (f) phenylalanine 556 of the F. novicida decarboxylase (SEQ ID NO: 198); and (g) leucine 560 of the F. novicida decarboxylase (SEQ ID NO: 198). In an exemplary embodiment, the modified decarboxylase enzyme is derived from a corresponding unmodified decarboxylase that is at least about 65%, 70%, 75%, 80%, 85%, 90%, 91%, 92%, 93%, 94%, 95%, 96%, 97%, 98%, 99%, or 99.5% identical to a polypeptide selected from SEQ ID NOs 1-214. In yet another exemplary embodiment, said spent yeast biocatalyst has been engineered to comprise at least one nucleic acid molecule encoding a polypeptide with keto-isovalerate decarboxylase (KIVD) activity, wherein said spent yeast biocatalyst comprises at least one nucleic acid molecule encoding a modified decarboxylase, wherein said decarboxylase has one or more modifications or mutations at positions corresponding to amino acids selected from: (a) phenylalanine 292 of the S. cerevisiae PDC1 (SEQ ID NO: 241); (b) threonine 388 of the S. cerevisiae PDC1 (SEQ ID NO: 241); (c) alanine 392 of the S. cerevisiae PDC1 (SEQ ID NO: 241); (d) serine 408 of the S. cerevisiae PDC1 (SEQ ID NO: 241); (e) valine 410 of the S. cerevisiae PDC1 (SEQ ID NO: 241); (f) isoleucine 476 of the S. cerevisiae PDC1 (SEQ ID NO: 241); (g) glutamine 552 of the S. cerevisiae PDC1 (SEQ ID NO: 241); and (h) threonine 556 of the S. cerevisiae PDC1 (SEQ ID NO: 241). In an exemplary embodiment, the modified decarboxylase enzyme is derived from a corresponding unmodified decarboxylase that is at least about 65%, 70%, 75%, 80%, 85%, 90%, 91%, 92%, 93%, 94%, 95%, 96%, 97%, 98%, 99%, or 99.5% identical to a wild-type pyruvate decarboxylase. In one embodiment, the wild-type, unmodified pyruvate decarboxylase is obtained from a yeast microorganism. In a further embodiment, the wild-type, unmodified pyruvate decarboxylase is obtained from a yeast microorganism classified into a genera selected from the group consisting of Saccharomyces, Kluyveromyces, Candida, Pichia, Issatchenkia, Debaryomyces, Hansenula, Pachysolen, Yarrowia, Schizosaccharomyces, Tricosporon, Rhodotorula, and Myxozyma. In another further embodiment, the wild-type, unmodified pyruvate decarboxylase is obtained from a Saccharomyces yeast. In an exemplary embodiment, the wild-type, unmodified pyruvate decarboxylase is obtained from Saccharomyces cerevisiae. In another exemplary embodiment, the wild-type, unmodified pyruvate decarboxylase is PDC1 (SEQ ID NO: 241), PDC5 (SEQ ID NO: 242), or PDC6 (SEQ ID NO: 243) of S. cerevisiae. In yet another exemplary embodiment, the wild-type, unmodified pyruvate decarboxylase is selected from SEQ ID NOs: 244-251. In additional embodiments, the spent yeast biocatalyst comprises a deletion or disruption of one or more endogenous pyruvate decarboxylase gene(s).

In certain additional embodiments, the DDG comprising a spent yeast biocatalyst of the present invention comprise at least one additional product selected from the group consisting of unconsumed feedstock solids, nutrients, proteins, fibers, and oils.

In another aspect, the present invention provides a method for producing DDG derived from a fermentation process using a yeast biocatalyst (e.g., a recombinant yeast microorganism of the present invention), said method comprising: (a) cultivating said yeast biocatalyst in a fermentation medium comprising at least one carbon source; (b) harvesting insoluble material derived from the fermentation process, said insoluble material comprising said yeast biocatalyst; and (c) drying said insoluble material comprising said yeast biocatalyst to produce the DDG.

In certain additional embodiments, the method further comprises step (d) of adding soluble residual material from the fermentation process to said DDG to produce DDGS. In some embodiments, said DDGS comprise at least one additional product selected from the group consisting of unconsumed feedstock solids, nutrients, proteins, fibers, and oils.

This invention is further illustrated by the following examples that should not be construed as limiting. The contents of all references, patents, and published patent applications cited throughout this application, as well as the Figures and the Sequence Listings, are incorporated herein by reference for all purposes.

### Example 1

### Identification of High-Performance Polypeptides with KIVD Activity

The purpose of this example is to show how high-performance polypeptides with keto-isovalerate decarboxylase (KIVD) activity were identified. More specifically, this example describes the development of a bioinformatics method to identify proteins which have KIVD (ketoisovalerate decarboxylase) activity but little to no PDC (pyruvate decarboxylase) activity.

### Background

Misannotation of DNA and protein sequences is the assignment of an erroneous functional description to a sequence whose function has not been experimentally determined. The primary source of misannotation is using simple sequence comparison to assign function. With the advent of next generation sequencing technology and the resulting rapid release of new genome sequences, there has been a steady increase in misannotation. Levels of misannotation for over 25% of protein super-families in one or more databases have been observed (Schnoes et al., 2009, PloS Comput Biol. 5: e1000605).

To diminish the level of misannotation, it is necessary to use multiple sequence alignments and apply a phylogenetic approach to determine the relationship between a sequence in question and those that have been characterized. This should include both those sequences that have been shown to encode a given function as well as those that encode related functions. This allows for possible boundaries of a given function to be defined.

### Polypeptide Identification

To identify genes encoding polypeptides with KIVD activity, the sequences of various proteins of interest listed in Table 2 below were used as a starting point.

TABLE 2
Proteins from KIVD/IPDC/PDC Families*.

| Species | Definition | Abbr | Accession | PubMed ID |
|---|---|---|---|---|
| Enterobacter cloacae | indolepyruvate decarboxylase | ipdC_Ecl | AAG00523.2 | 18757531 |
| Paenibacillus polymyxa | indole-3-pyruvate decarboxylase | ipdC_Pp | ABV24338.1 | 18667851 |
| Azospirillum brasilense | Indole-3-pyruvate decarboxylase | ipdC_Abr | P51852.1 | 8202090 |
| Lactococcus lactis | alpha-ketoisovalerate decarboxylase | kivd_Ll | CAG34226.1 | 15358422 |
| Azospirillum lipoferum | Indole-3-pyruvic acid decarboxylase | ipdC_Ali | Q93RB7 | 11440156 |
| Pantoea agglomerans | Indolepyruvate decarboxylase | ipdC_Pa | P71323 | 11248099 |
| Saccharomyces cerevisiae | pyruvate decarboxylase | pdc1_Sc | CAA97573.1 | various |
| Zymobacter palmae | pyruvate decarboxylase | pdc_Zp | AAM49566.1 | 12039744 |
| Zymomonas mobilis | pyruvate decarboxylase | pdc_Zm | CAA42157.1 | 3546263 |

*KIVD = ketoisovalerate decarboxylase; IPDC = indole pyruvate decarboxylase; PDC = pyruvate decarboxylase.

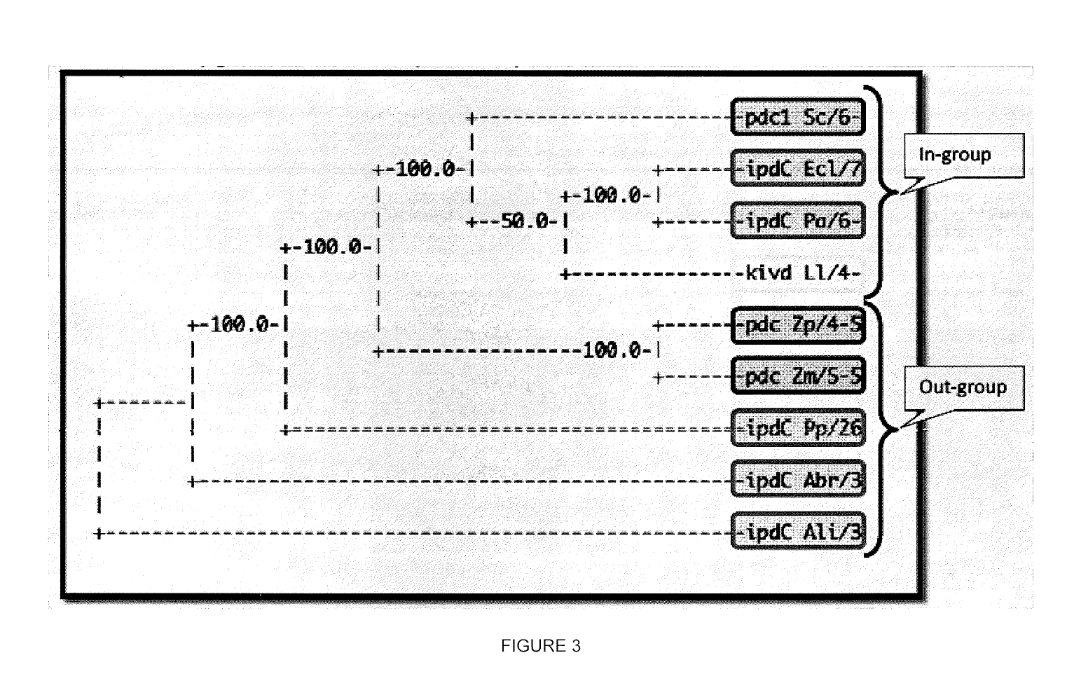

For a preliminary examination the above sequences were aligned using clustalw2 (version 2.0.12). The alignment was examined with Jalview and areas of insertions and deletions were eliminated with the exception of those that were clearly specific to a lineage or sequence. The Phylip (version 3.69) programs ‘protdist’ and ‘neighbor’ were used to create an un rooted neighbor joining tree with boot strap values generated using the ‘seqboot’ and ‘consense’ programs (100 replicates). Boot strap values are shown for branch points (FIG. 3). It appears that IPDC (indole-pyruvate decarboxylase) arose at least twice and that the PDC (pyruvate decarboxylase) line may have given rise to KIVD (keto-isovalerate decarboxylase and one of the two IPDC (indole-pyruvate decarboxylase group).

Database Search Using Query Sequences:

The characterized sequences are used to search a protein or DNA sequence database (i.e., target database) using a sequence comparison program appropriate for the query sequence and the database being searched. The preferred approach is to compare protein sequences of the GenBank ‘nr’ (nonredundant) database using the blastp algorithm (version 2.2.23) with an expect value cutoff of 0.1. Sequences from the target database that are matched are referred to as “hits” and processed further.

In-Group and Out-Group Analysis:

As shown in FIG. 3, the sequences for the S. cerevisiae pyruvate decarboxylase, the E. cloacae indole-pyruvate decarboxylase, the P. agglomerans indole-pyruvate decarboxylase, and the L. lactis keto-isovalerate decarboxylase (herein called the “in-group”) are more closely related to each other than any of these four are related to the Z. palmae or Z. mobilis pyruvate decarboxylases (herein called the “out-group”). Sequence comparison using the blastp algorithm revealed that the lowest in-group bit score was 302. For comparisons between the in-group and out-group, no score was higher than 270. Finally the difference between the maximum non-self bitscore for the in-group comparison and the max bit score was never less than 133.

To further refine the set of hit sequences for multiple sequence alignment, only those with a maximum bit score to members of the in-group of 300 or greater and with a maximum out-group bit score that is 100 or more less than the maximum in-group bit score were worked with further. In other words, sequences for alignment preferably had a blast bit score of 300 or greater to one of the four members of the in group and having a maximum bit score to in group members that is at least 100 points higher than the maximum score to the out-group members.

To facilitate subsequent alignment procedures, hit sequences with lengths not falling between 450 and 650 amino acids, or that do not begin with a methionine may be eliminated.

Hit Groups from the In-Group Analysis: Also “hit” sequences may be grouped based on a 65% identity cutoff such that any member of a resulting group shares 65% identity with at least one other member of that group and that no member from different groups share 65% or greater identity based on standard blastp comparison. A single representative sequence from each group was chosen based on length with the longest sequence being chosen and if two or more sequences are of the maximum length one is chosen arbitrarily. All “hit” sequences were placed into one of several “hit groups” and given a reference identifier.

### Results

Phylogenetic Tree:

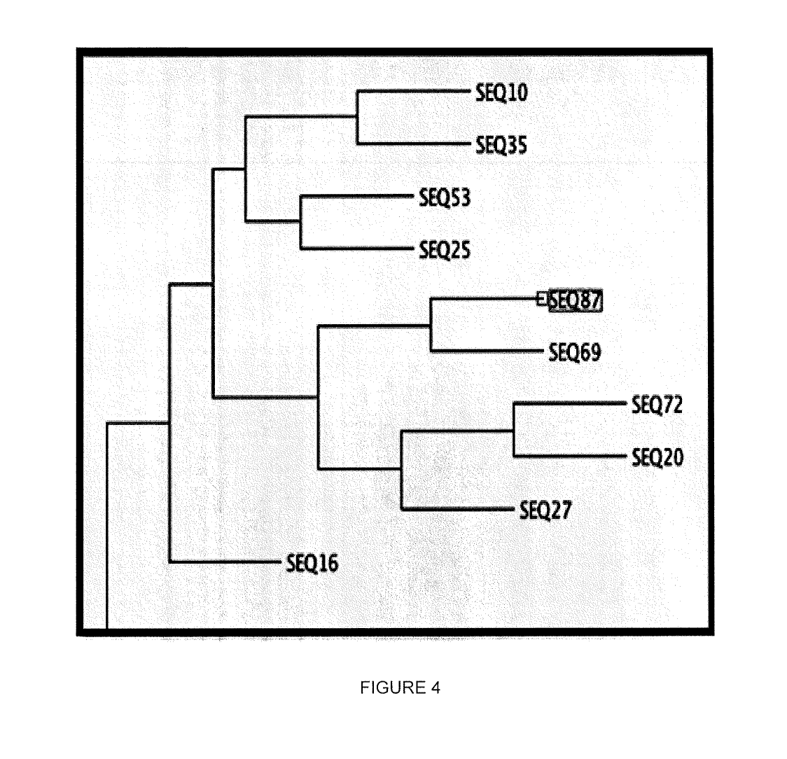

To create a phylogenetic tree, the representative sequences for each of the “hit groups” are first aligned using a multiple sequence alignment software preferably clustalw2 (version 2.0.12). Sequence alignments are then hand edited with sequences being discarded if they cause the introduction of a large number of gaps in the overall alignment. Positions in regions with large numbers of gaps are preferably deleted from the sequence alignment except where they are clearly specific to a lineage or sequence. The resulting edited alignment is preferably no less than 450 amino acids in length. Phylip (version 3.69) programs ‘protdist’ and ‘neighbor’ were used to create an un rooted neighbor joining tree with boot strap values generated using the ‘seqboot’ and ‘consense’ programs (1000 replicates)—this analysis allowed for the creation of an extended KIVD/IPDC/PDC protein family (see FIG. 4 of U.S. Provisional Application Ser. No. 61/512,810, which is herein incorporated by reference).

KIVD Proteins:

Sequences falling within the same clade as the L. lactis kivD (GenBank Accession No: CAG34226.1) or its representative, and that do not contain sequences associated with other activities are likely to also have KIVD activity. The likelihood a branch will have KIVD activity increases the closer a given branch is to a branch carrying KIVD. The tree in FIG. 4 can be used to further illustrate this point. The hit group “SEQ87” represents the L. lactis kivD (GenBank Accession No: CAG34226.1, SEQ ID NO: 197). Based upon this analysis, the hit group “SEQ69” would be more likely to have KIVD activity than the more distant hit group “SEQ16.”

### Example 2

### Structure-Based Sequence Determinants of Polypeptides with KIVD Specificity

The purpose of this example is to show how high-performance polypeptides with keto-isovalerate decarboxylase (KIVD) activity were identified using structure-based criteria for predicting the specificity of a polypeptide sequence homolog. Polypeptides exhibiting high keto-isovalerate decarboxylase (KIVD) activity with reduced pyruvate decarboxylase (PDC) activity were identified.

Polypeptide Identification:

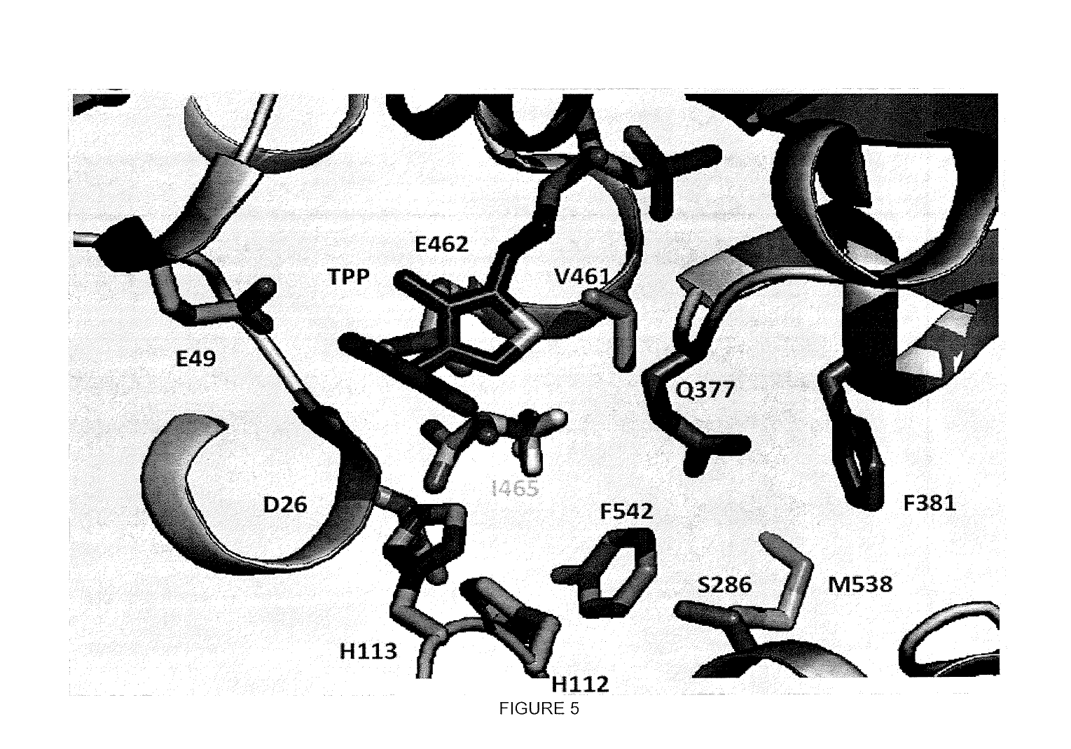

Protein database BLAST searches revealed several significant hits. Notably, the crystal structures 2vbf (FIG. 5) and 2vbg correspond to the Branched-Chain Keto Acid Decarboxylase from L. lactis (KdcA), an enzyme which exhibits keto-isovalerate decarboxylase activity—crystal structures are available from the Protein Data Bank (“PDB”). KdcA is 88% identical to KivD from L. lactis. 1ovm is an indolepyruvate decarboxylase from E. cloacae (Ec_IPDC, 40% identity to KivD from L. lactis). There are a number of structures of the PDC from yeast (S. cerevisiae PDC, 37% identity to KivD from L. lactis) including various mutants: 1qpb, 2w93, 2vk8, 1pvd, 1pyd, 2vk1. 2vjy is PDC from K. lactis (KI_PDC, 37% identity to KivD from L. lactis). 2vbi is a PDC from A. pasteurianus (Ap_PDC, 32% identity to KivD from L. lactis). Besides the yeast, the other well-studied PDC is from Z. mobilis (Zm_PDC, 33% identity to KivD from L. lactis): 2wva, 3oe1, 1zpd.

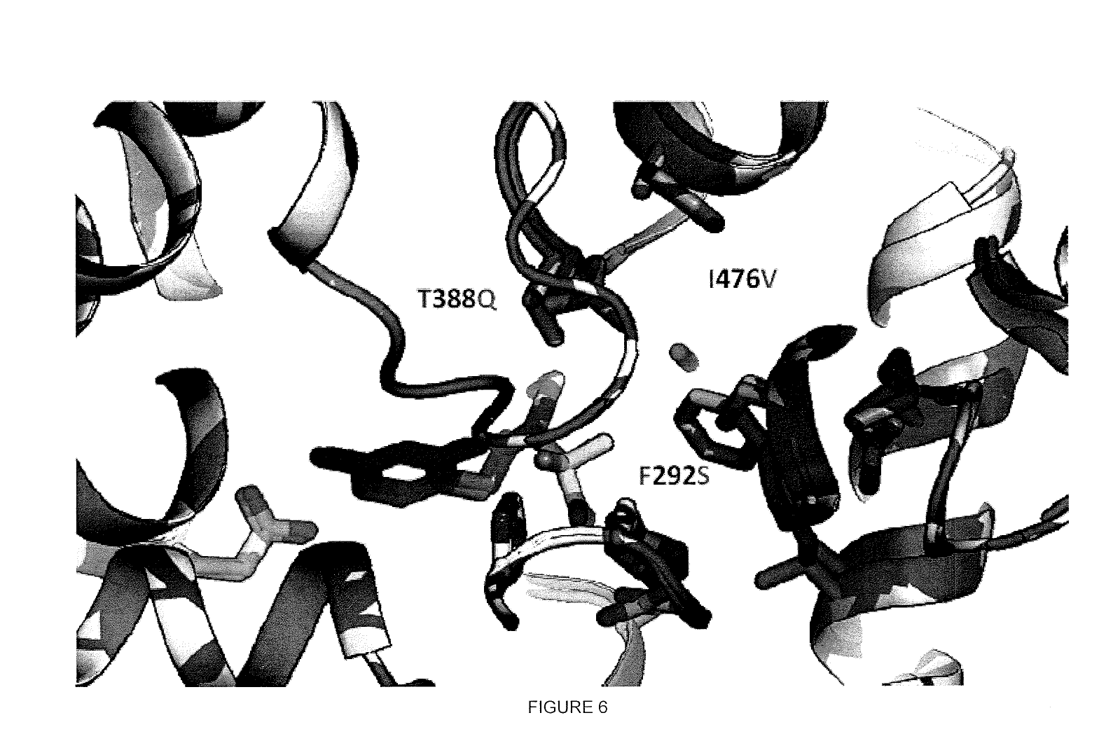

Comparison between the Sc_PDC and KdcA was used to identify “specificity residues” involved in discriminating between pyruvate and keto-isovalerate (FIG. 6).

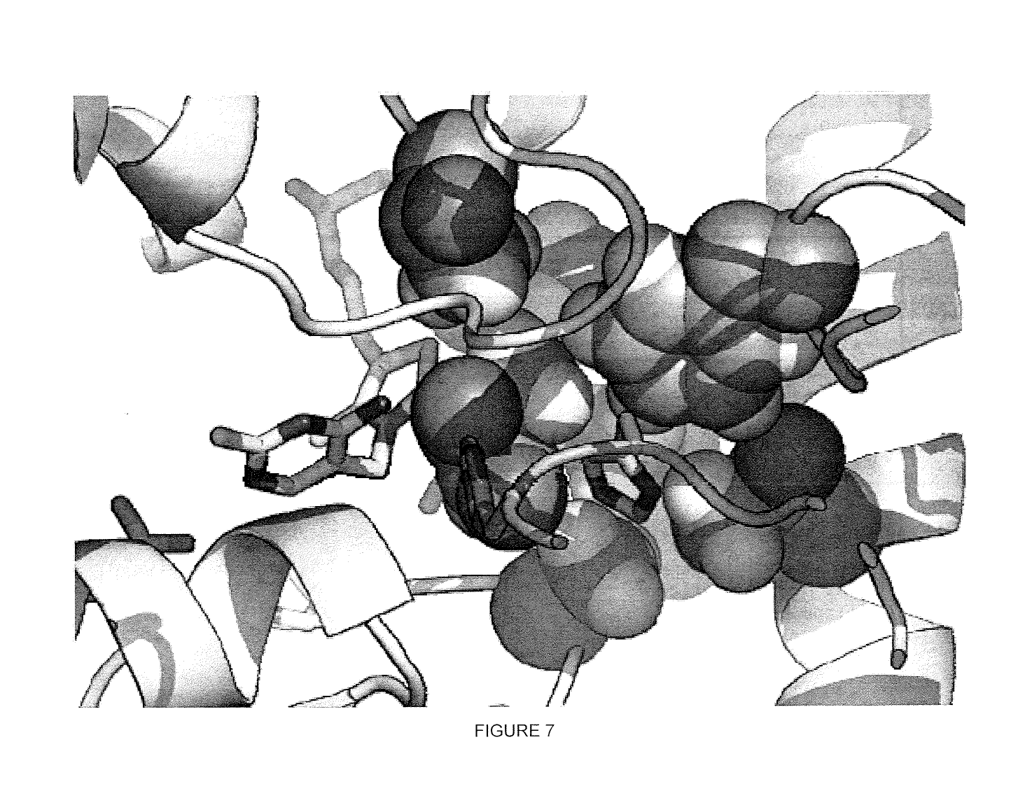

A spacefilling model for Sc_PDC illustrates a tight fit between pyruvate and the substrate-binding pocket is achieved (FIG. 7).

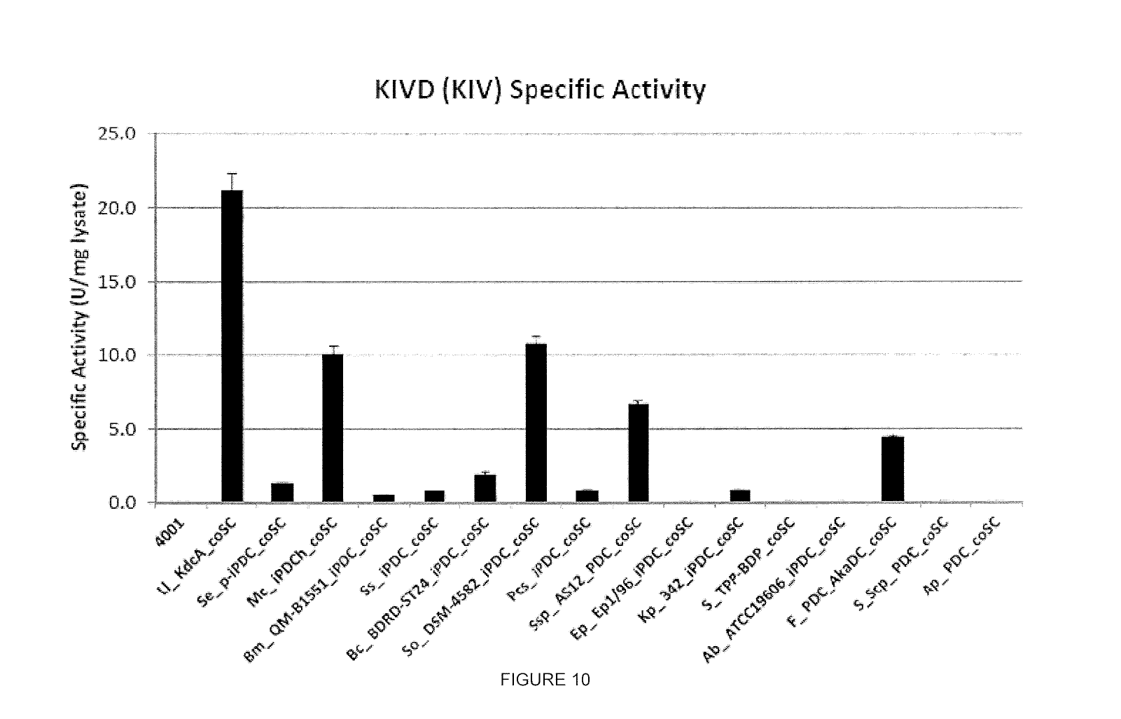

The sequence alignment between the L. lactis keto-isovalerate decarboxylases KivD and KdcA, and a homology model for the L. lactis KivD indicate that KdcA is an appropriate structural model for the L. lactis KivD. The two active sites are completely conserved amongst the two proteins (see FIG. 10 of U.S. Provisional Application Ser. No. 61/512,810, which is herein incorporated by reference). Importantly, the catalytic residues, D26, H112, H113, G402, and E462 are completely conserved. Likewise, the specificity residues, S286, Q377, F381, V461, I465, M538, and F542, are also conserved (see FIG. 10 of U.S. Provisional Application Ser. No. 61/512,810, which is herein incorporated by reference). This allowed for the identification of a KIVD substrate specificity motif, identified herein as “SQFVIMF” (SEQ ID NO: 237), which corresponds to the specificity residues, S286, Q377, F381, V461, I465, M538, and F542 of the L. lactis KIVD of SEQ ID NO: 197.

Once a set of specificity-determining sites had been identified, a blast search against the non-redundant protein sequence database was performed. The resulting 1000 sequences extend down to 25% sequence identity. This list was further filtered by eliminating hits in which 5 critical catalytic residues are absent: D26, H112, H113, G402, and E462. This excluded from consideration phenylpyruvate decarboxylase sequences (which lack one of the catalytic glutamic acids). For each of the remaining 508 sequences, the amino acids matched in the blast alignment to the L. lactis KivD specificity-determining residues: S286, Q377, F381, V461, I465, M538, and F542, were aligned. Each candidate sequence was classified according to the first true Boolean test (where M (Zm_PDC) refers to the number of “specificity residues” that match Zm_PDC). The following cutoffs were used to identify polypeptides with highly specific KIVD activity:

1. If M(Zm_PDC)>6, classify the sequence “Specific PDC”.

2. If M(Sc_PDC)>6, classify the sequence “Non-specific PDC”.

3. If M(LI_KIVD)>6, classify the sequence “KIVD”.

4. If M(Ec_IPDC)>6, classify the sequence “IPDC”.

5. If M(LI_KIVD)>2 and M(Zm_PDC)<3 and M(Sc_PDC)<3 and V461 is conserved, classify the sequence “Potential KIVD”

6. If M(LI_KIVD)<3 and M(Ec_IPDC)<3 and (M(Sc_PDC)>4 or M(Zm_PDC)>4), classify the sequence “Potential PDC”

7. If Val461 is replaced with Ile and Gln377 is replaced with a beta branched amino acid (Val, Thr, Ile), classify the sequence “Unbranched” disfavoring a branched substrate)

8. Classify the sequence “Unknown”.

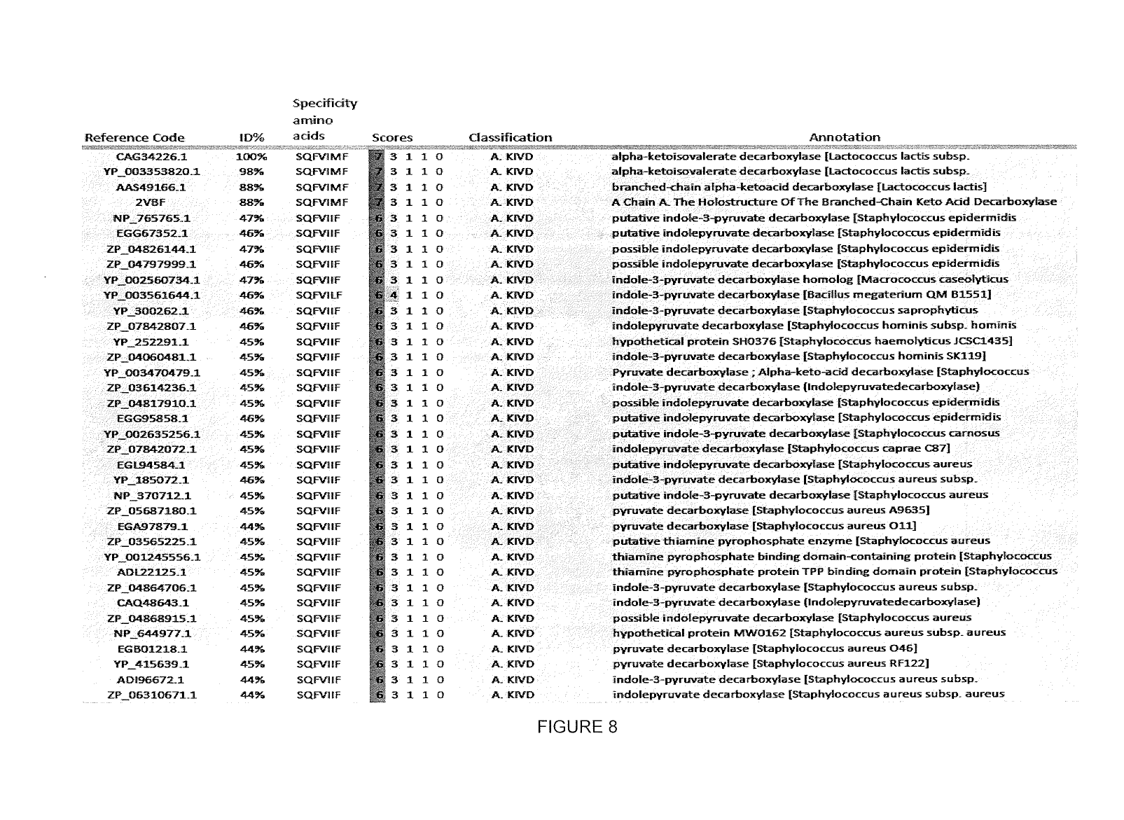

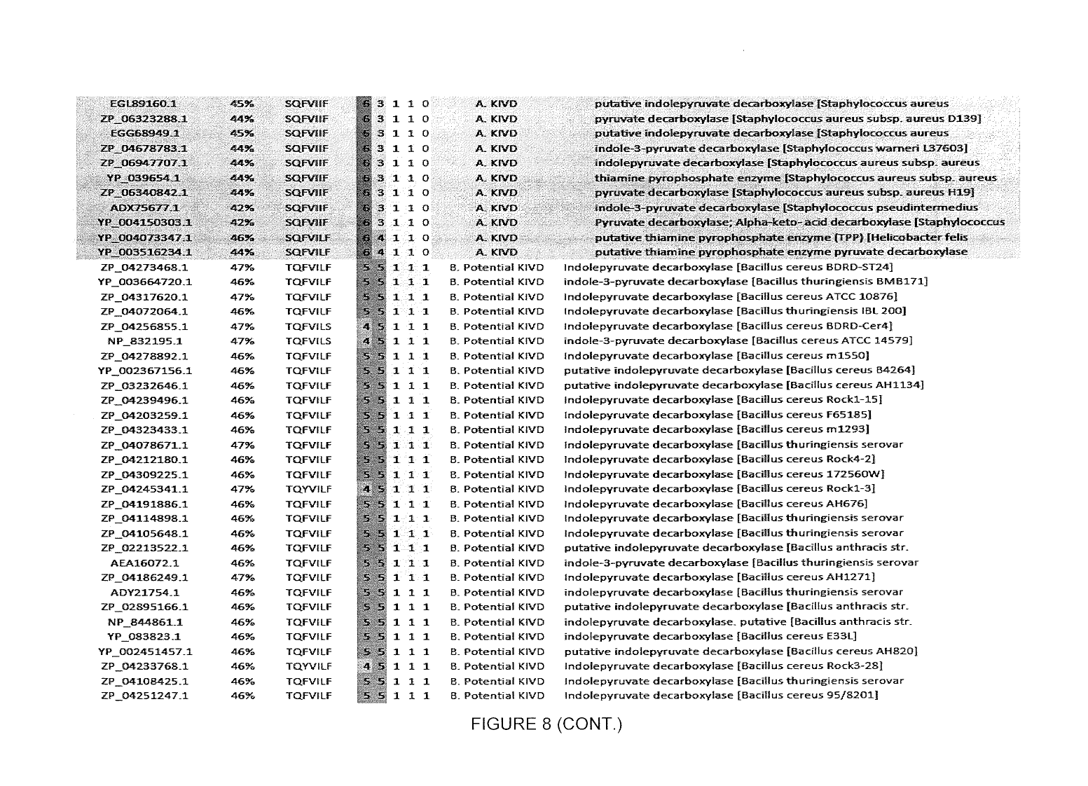

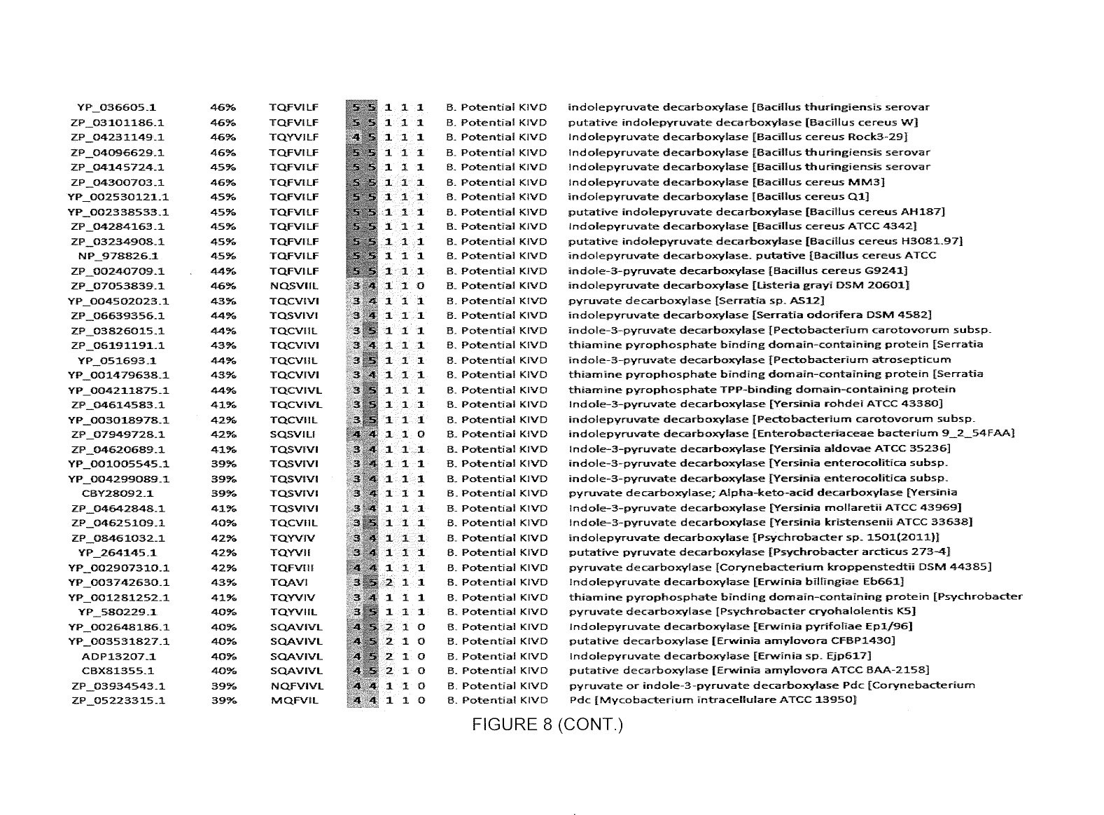

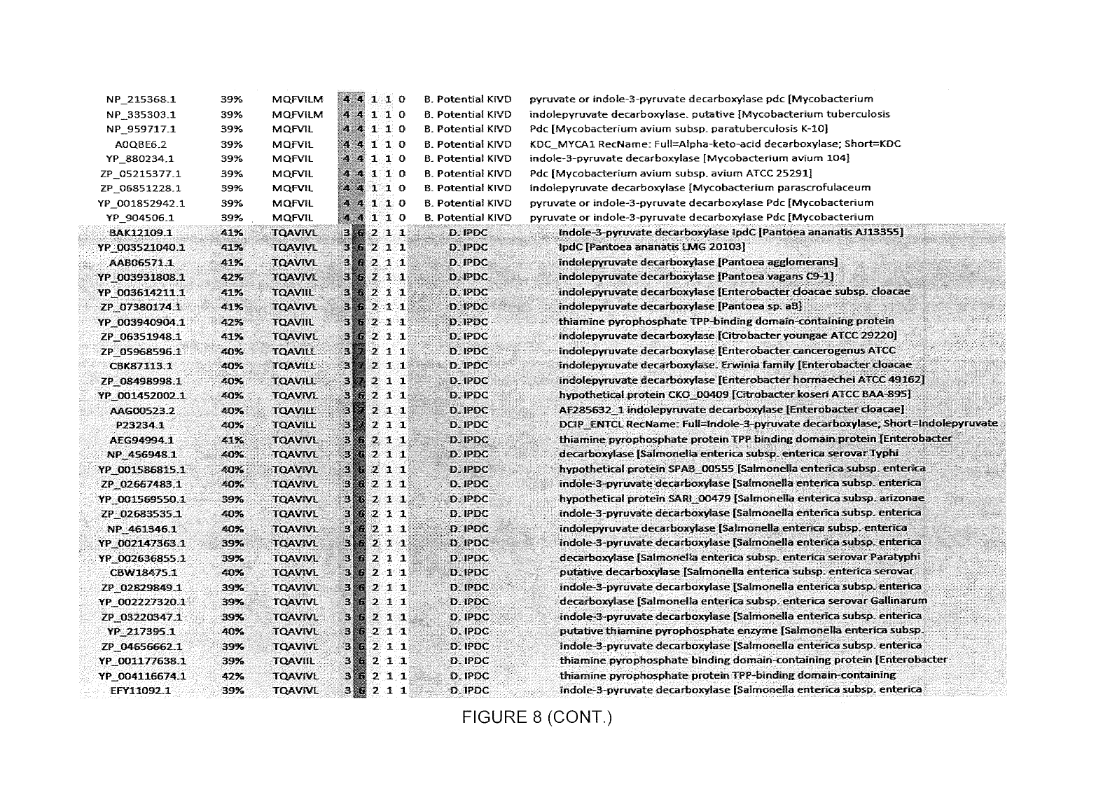

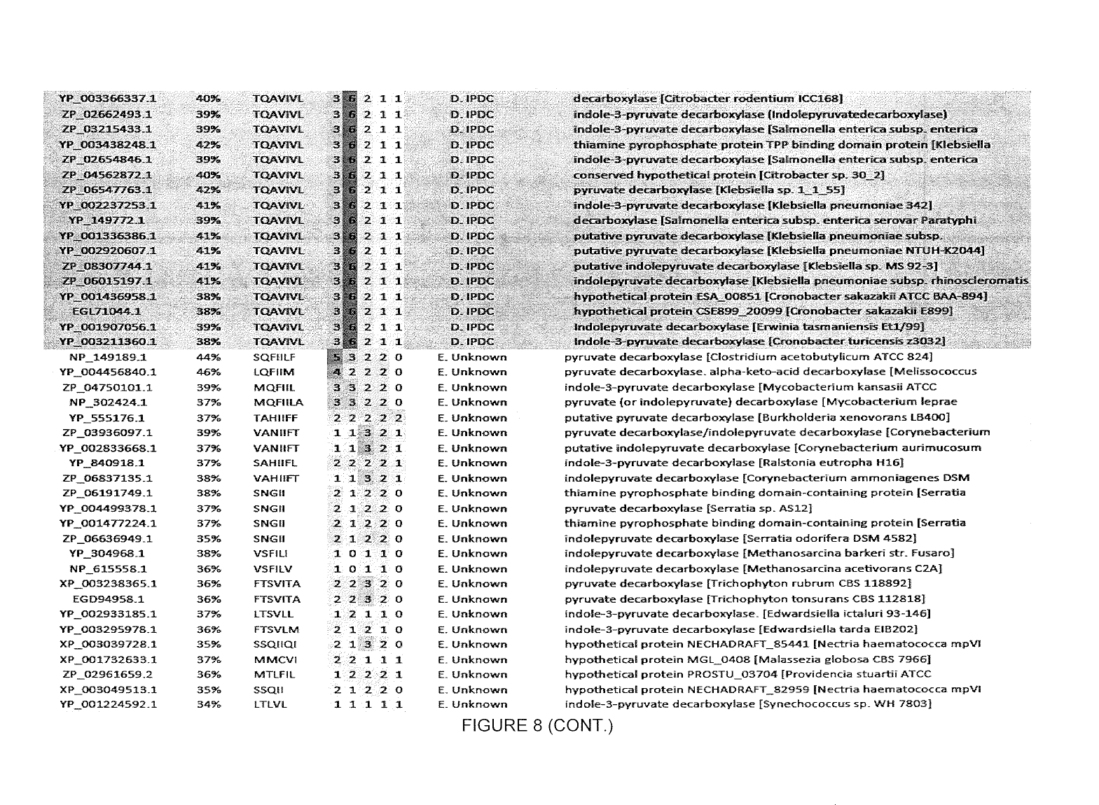

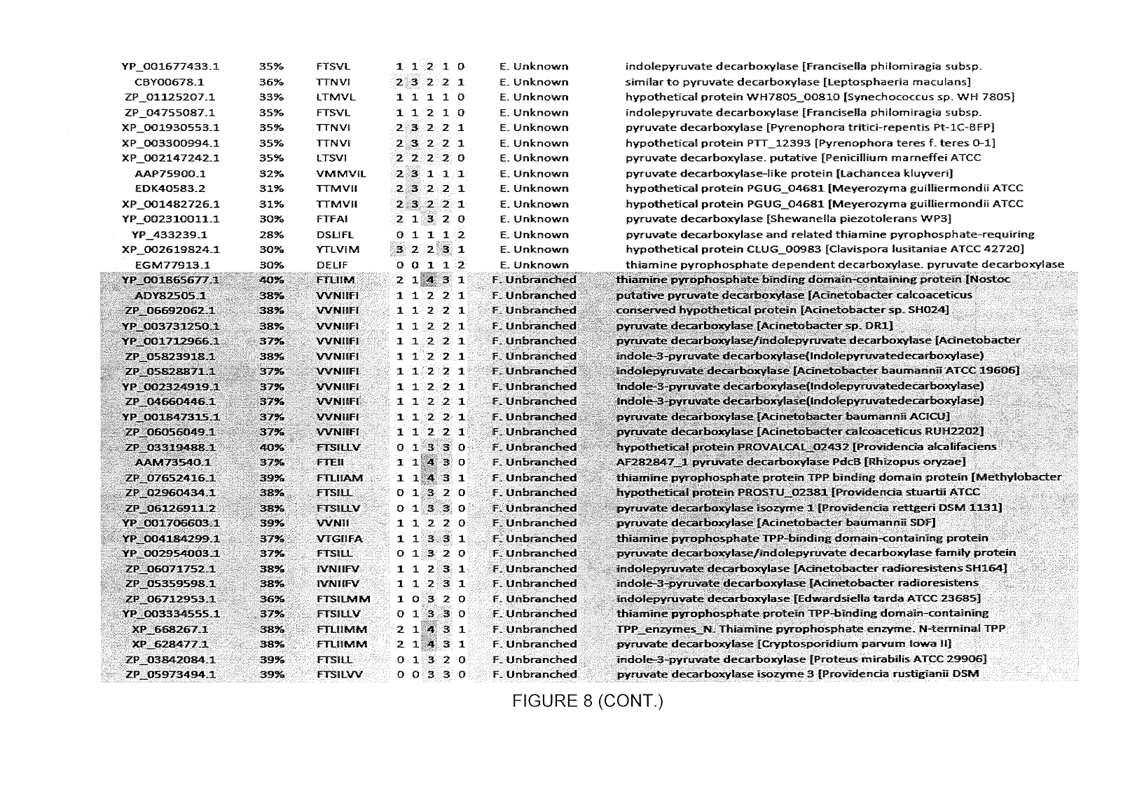

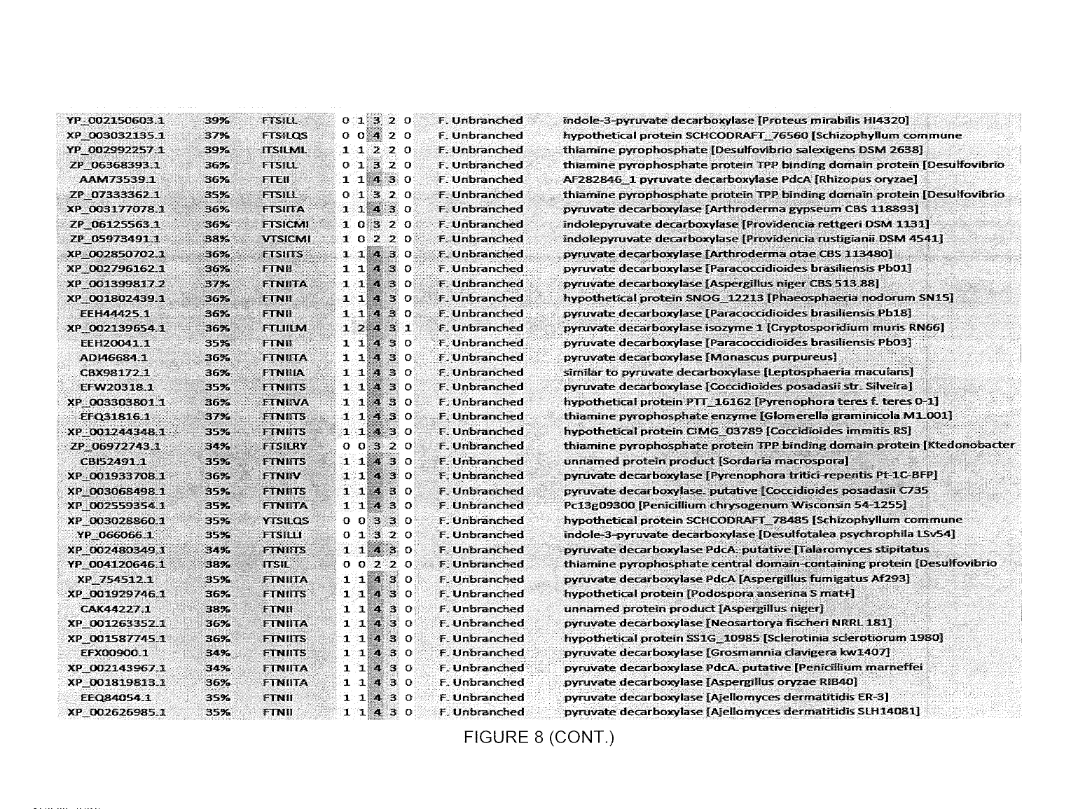

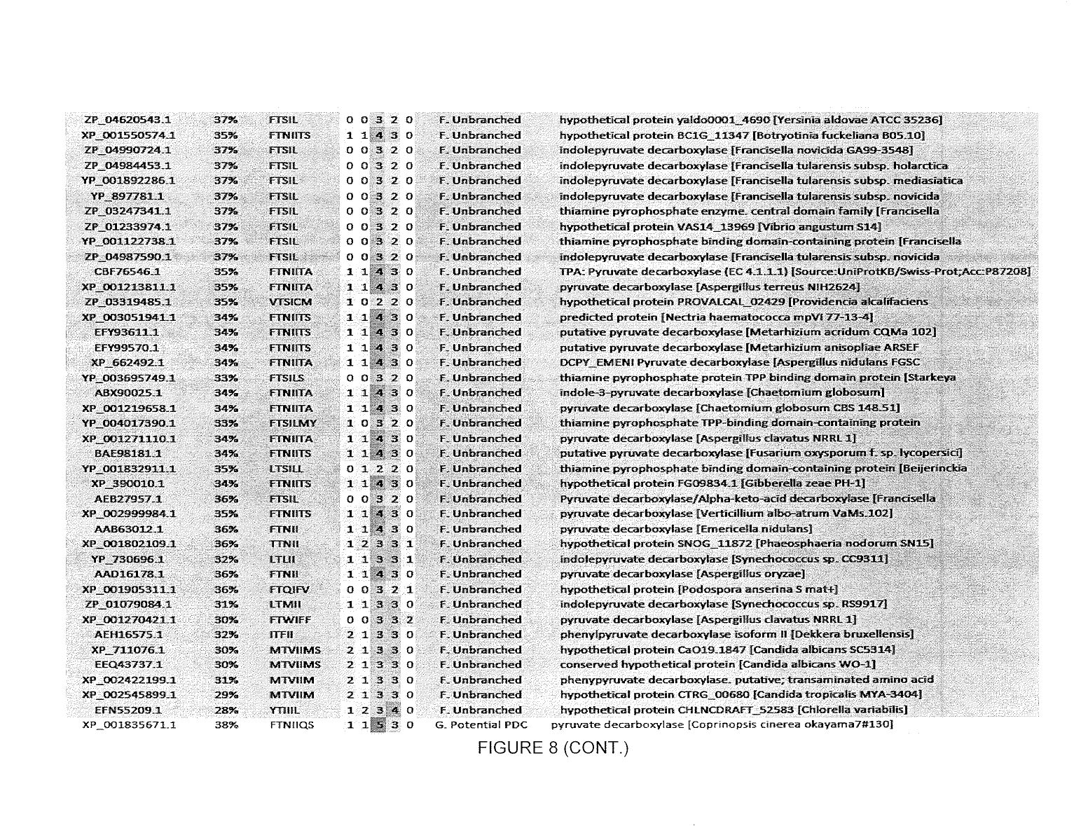

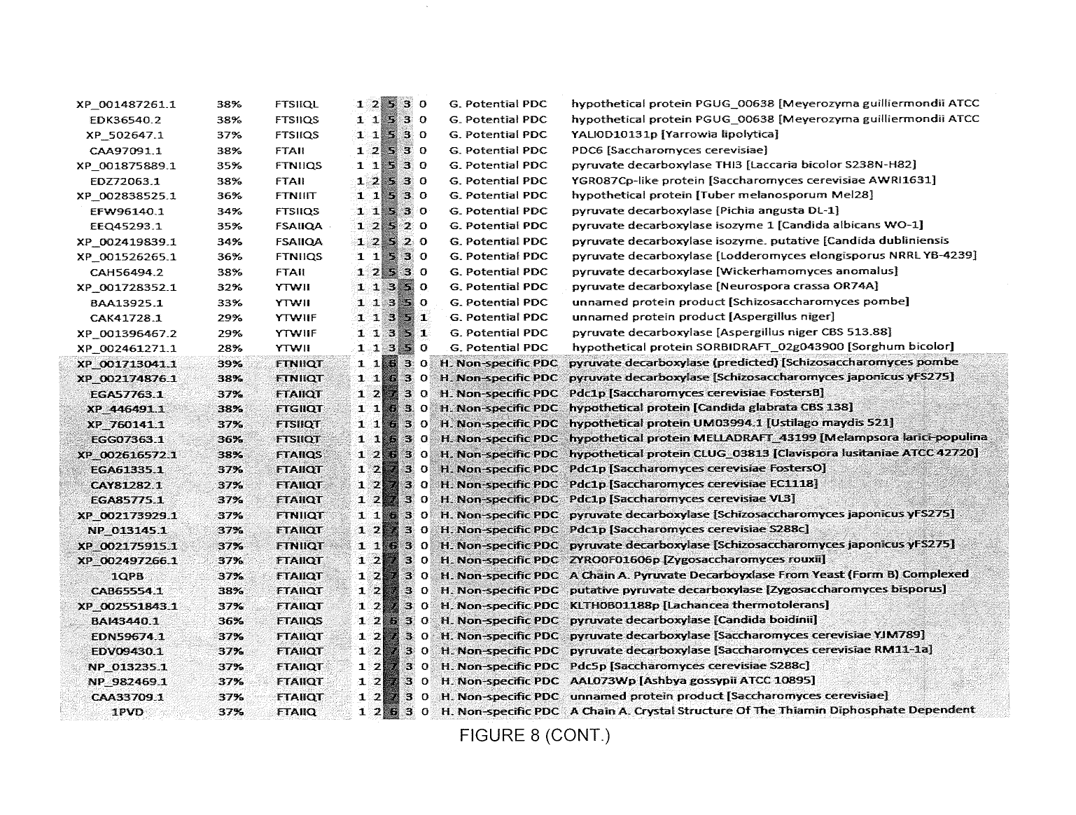

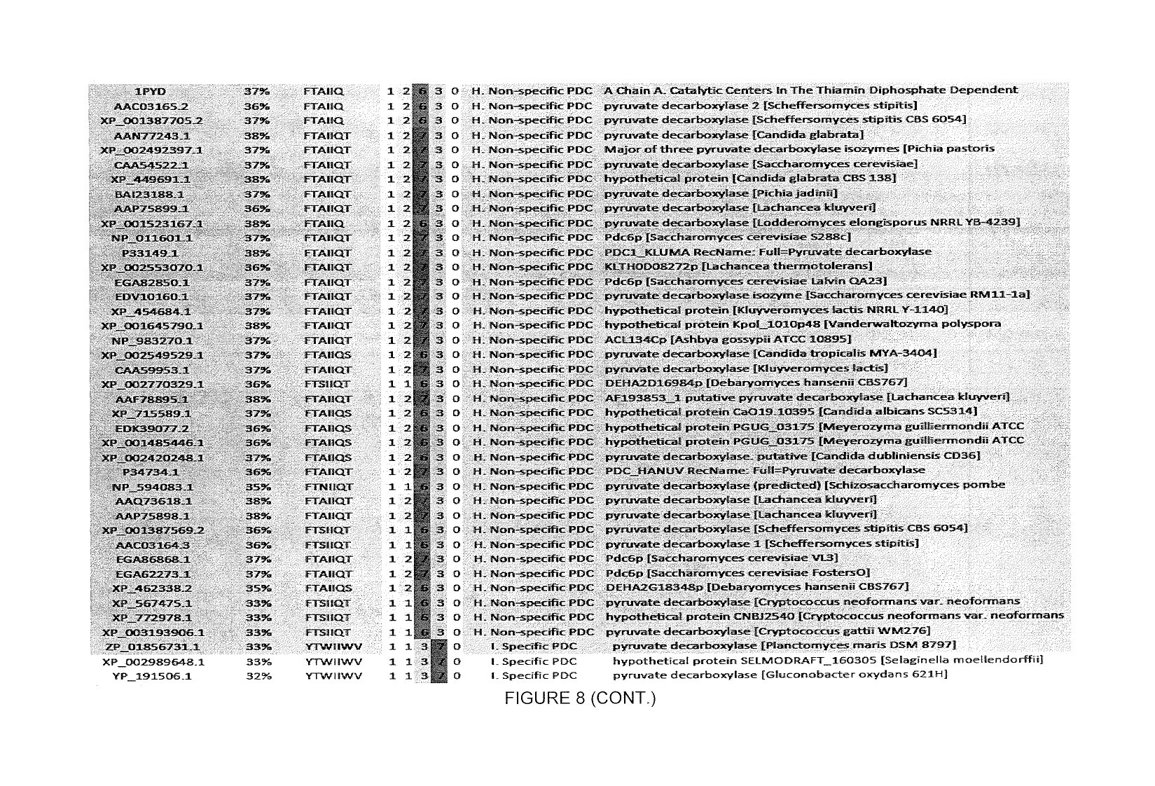

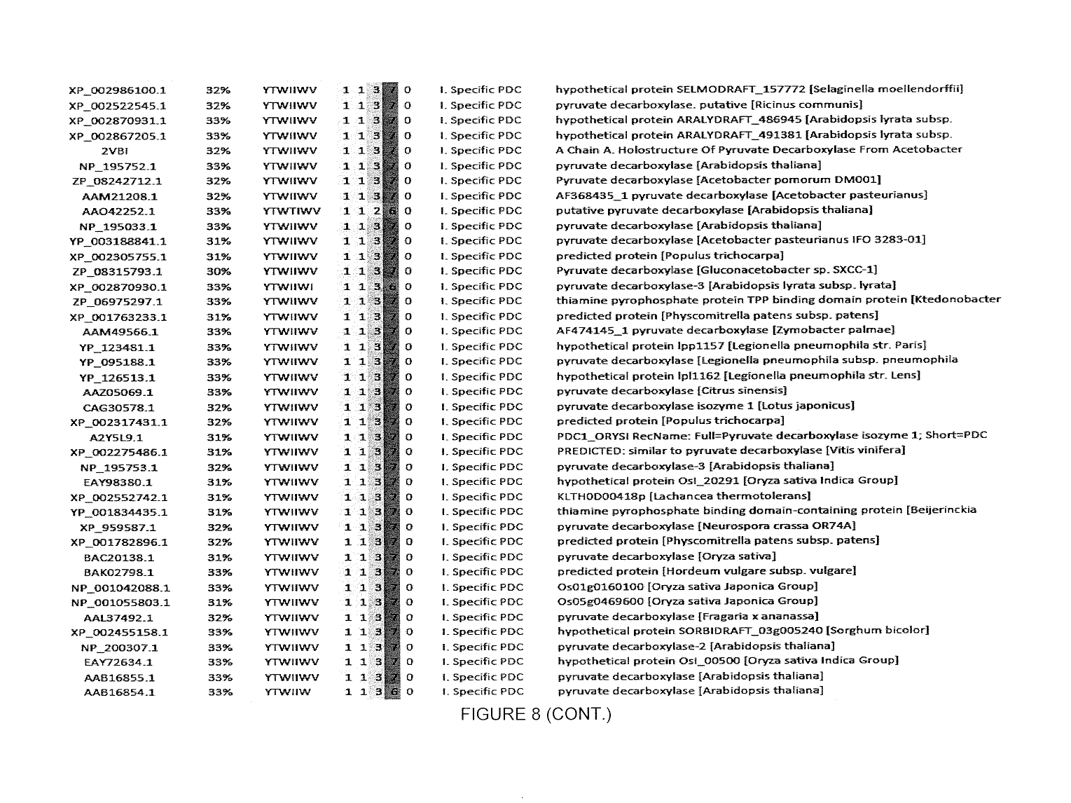

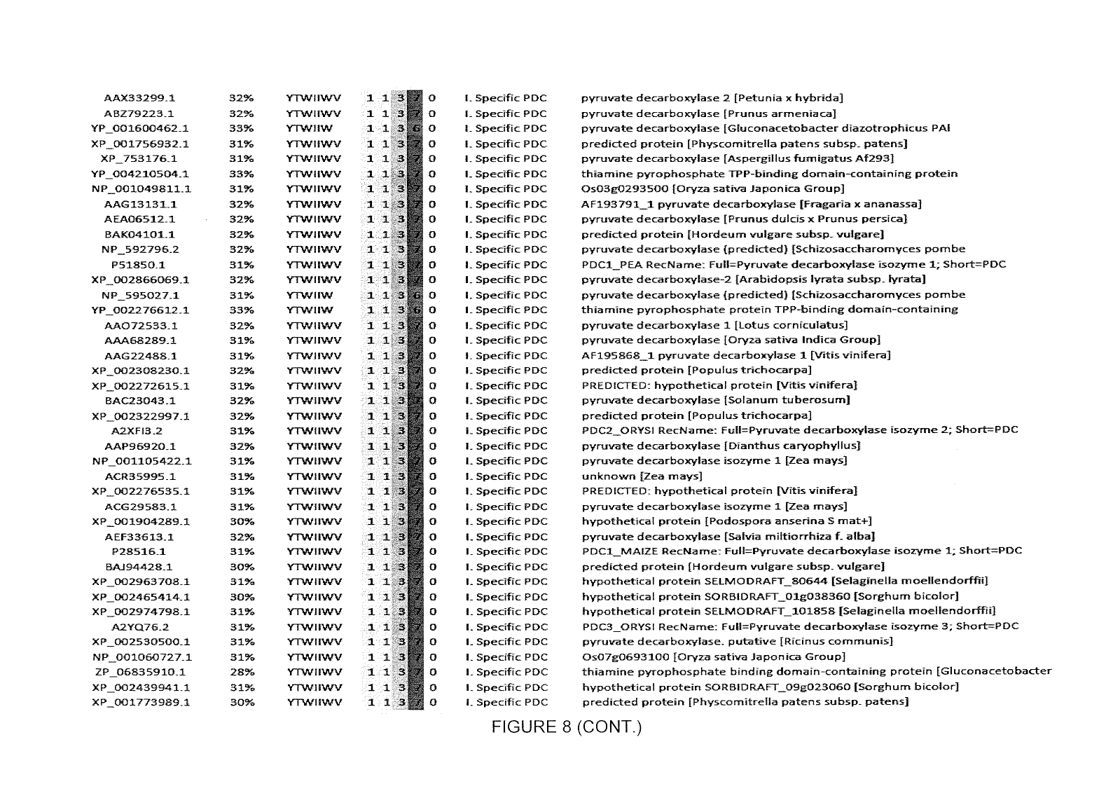

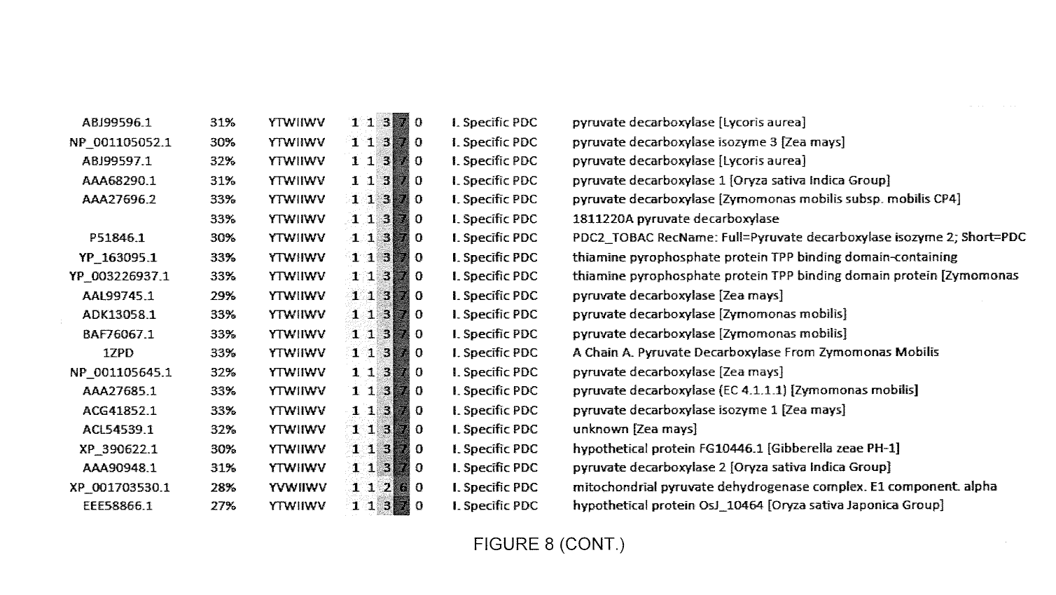

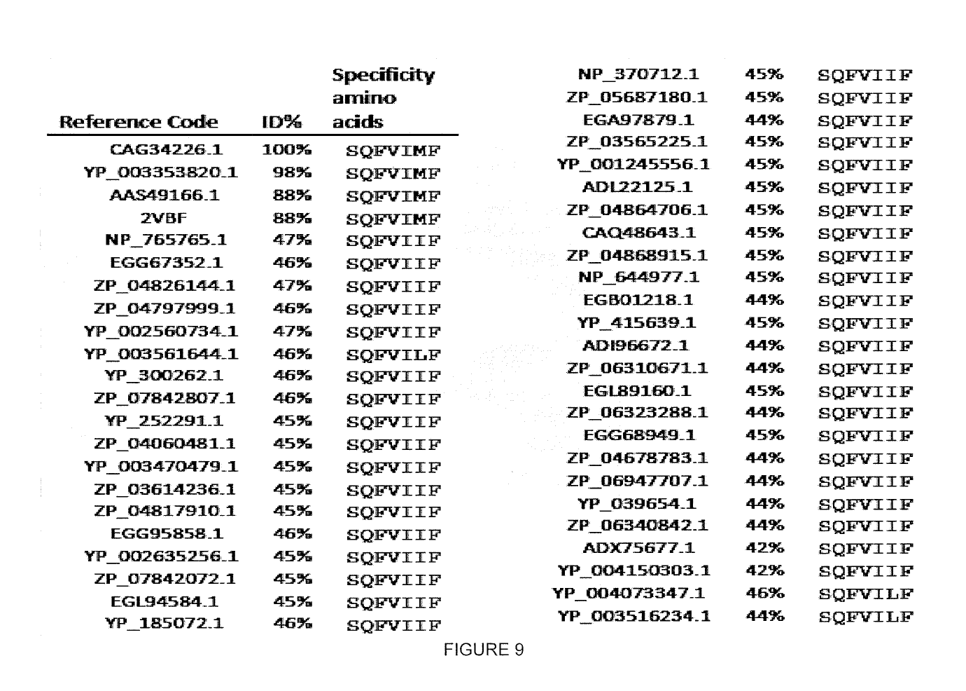

The classified sequences were sorted based upon likely specific KIVD activity (i.e., most likely KIVD on top, most likely PDC on bottom). This sort is illustrated in FIG. 8. Using the above-identified cutoffs, 47 sequences were classified as KIVDs (FIG. 9).

The sequences returned from BLAST analysis are largely annotated as pyruvate decarboxylases or indolepyruvate decarboxylases. The specificity analysis of active site residues described herein suggests that many of the latter may harbor keto-isovalerate decarboxylase (KIVD) activity.

### Example 3

### Evaluation of Decarboxylase Enzymes for KIVD Activity and Substrate Specificity

The purpose of this example is to show how a high degree of identity to the KIVD substrate specificity motif “SQFVIMF” identified in Example 2 is generally predictive of: (a) high KIVD activity; (b) reduced PDC activity; and (c) a high KIV/pyruvate activity ratio.

In this example, 16 different decarboxylases representing a cross-section of decarboxylases, with varying degrees of identity to the “SQFVIMF” motif were selected from FIG. 8 and examined through in vitro enzyme assays. Table 3 lists the decarboxylases in a decreasing order of substrate specificity towards KIV as compared to pyruvate based on a statistical scoring mechanism for amino acid residues constituting the “SQFVIMF” motif.

Experimental Design: All decarboxylases tested in this example were codon-optimized for expression in S. cerevisiae. Plasmids comprising the individual decarboxylase homologs were used to generate transformants of S. cerevisiae strain, GEVO4001 (“4001”). Transformants were grown in shake flasks overnight at 33° C. at 250 rpm. The following day, 3 ml cultures were used to inoculate 50 mL growth medium at OD₆₀₀ of 0.2 and incubated at 33° C. at 250 rpm for 24 hrs. Cell pellets (OD₆₀₀ of 20 per pellet) were prepared and measured for KIVD and PDC activities in cell lysates.

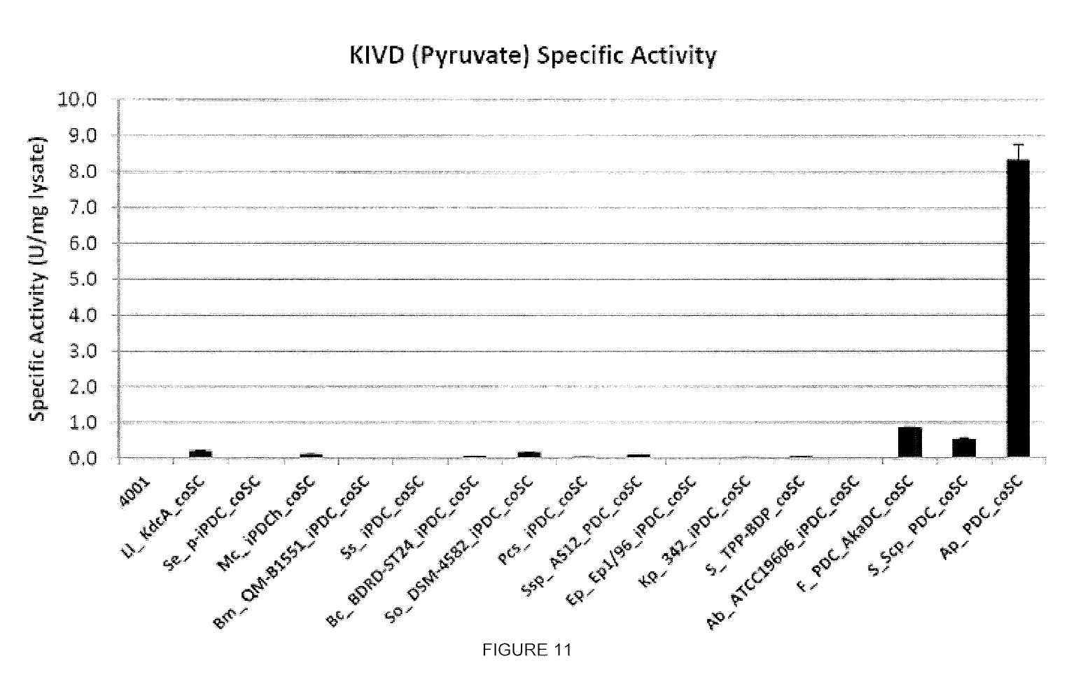

FIGS. 10 and 11 show KIVD and PDC specific activity for the indicated decarboxylases, generally arranged in a decreasing order of percent amino acid identity to the L. lactis KIVD of SEQ ID NO: 197, as well as a decreasing identity score to the predicted KIVD substrate specificity motif “SQFVIMF”.

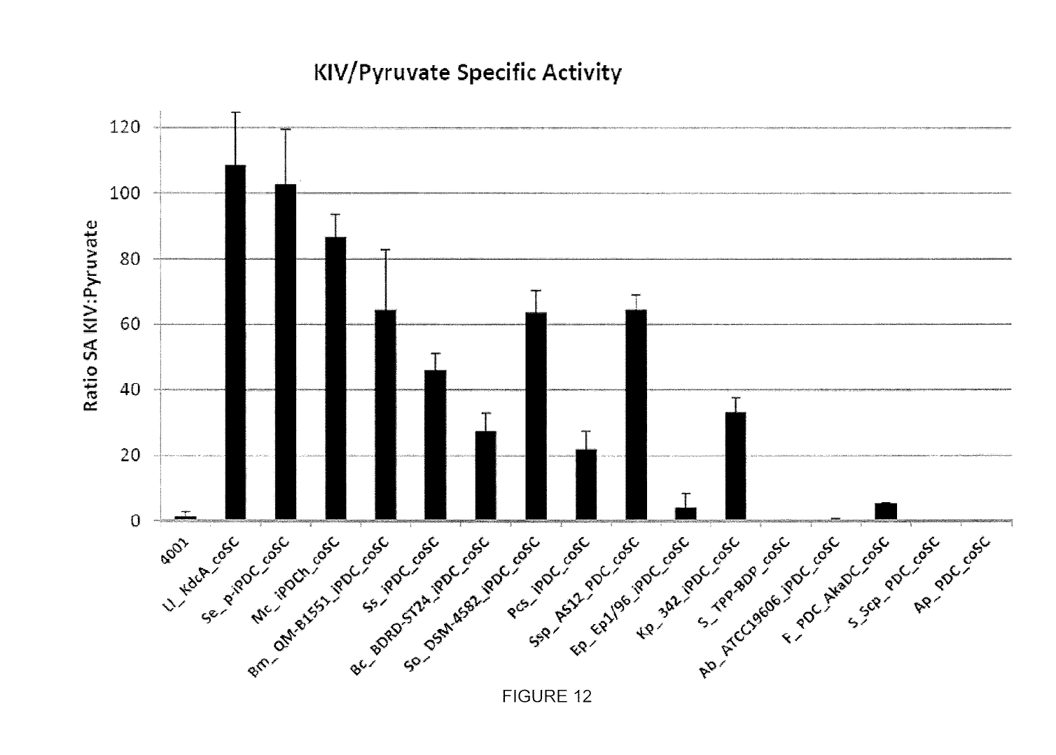

These data together suggest that the decarboxylases with a higher degree of identity to the predicted KIVD substrate specificity motif “SQFVIMF” tend to have higher KIVD activity and lower PDC activity. Conversely, decarboxylases with a higher PDC and lower specific KIVD activity exhibit a substrate specificity motif closer in identity to a predicted PDC substrate specificity motif “FTAIIQT” (SEQ ID NO: 238) as opposed to KIVD substrate specificity motif. A high KIV:Pyruvate activity ratio also seems to favor decarboxylase homologs with a higher degree of identity to the predicted KIVD motif as compared to the predicted PDC motif (FIG. 12). A notable exception is the decarboxylase derived from Francisella, which exhibited a substrate specificity score distinct from the identified KIVD substrate specificity motif.

TABLE 3
List of decarboxylase homologs with the indicated % protein identity (ID%)
relative to the L. lactis KIVD of SEQ ID NO: 197. Using protein structure
analysis as well as sequence alignment, the amino acid residues corresponding
to the identified likely specificity-determining residues (i.e., S286, Q377,
F381, V461, 1465, M538, and F542; “SQFVIMF”) were identified collectively as
a substrate specificity motif for IPDC, PDC1, PDC2, and PPDC. Each number
denotes the number of amino acid residues that each decarboxylase homolog
shares with the substrate specificity motif for KIVD, IPDC, PDC1, PDC2, and
PPDC. The profile of motif identity scores is used to
classify each decarboxylase homolog.

| | | | Specif- | | | | | | |
|---|---|---|---|---|---|---|---|---|---|
| | | | icity | | | | | | |
| | | | amino | | | | | | Classi- |
| Species | Gene | ID% | acids | kivd | ipdc | pdc1 | pdc2 | ppdc | fication |
| Lactococcus | Ll_KdcA_coSC | 88% | SQFVIMF | 7 | 3 | 1 | 1 | 0 | KIVD |
| lactis (KdcA) | | | (SEQ ID | | | | | | |
| | | | NO.: 252) | | | | | | |
| Staphylococcus | Se_p-iPDC_coSC | 47% | SQFVIIF | 6 | 3 | 1 | 1 | 0 | KIVD |
| epidermidis | | | (SEQ ID | | | | | | |
| | | | NO.: 253) | | | | | | |
| Macrococcus | Mc_iPDCh_coSC | 47% | SQFVIIF | 6 | 3 | 1 | 1 | 0 | KIVD |
| caseolyticus | | | (SEQ ID | | | | | | |
| | | | NO.: 254) | | | | | | |
| Bacillus | Bm_QM-B1551_iPDC_coSC | 46% | SQFVILF | 6 | 4 | 1 | 1 | 0 | KIVD |
| megaterium | | | (SEQ ID | | | | | | |
| QM B1551 | | | NO.: 255) | | | | | | |
| Staphylococcus | Ss_iPDC_coSC | 46% | SQFVIIF | 6 | 3 | 1 | 1 | 0 | KIVD |
| saprophyticus | | | (SEQ ID | | | | | | |
| | | | NO.: 256) | | | | | | |
| Bacillus cereus | Bc_BDRD-ST24_iPDC_coSC | 47% | TQFVILF | 5 | 5 | 1 | 1 | 1 | Potential |
| BDRD-ST24 | | | (SEQ ID | | | | | | KIVD |
| | | | NO.: 257) | | | | | | |
| Serratia | So_DSM-4582_iPDC_coSC | 44% | TQSVIVI | 3 | 4 | 1 | 1 | 1 | Potential |
| odorifera | | | (SEQ ID | | | | | | KIVD |
| DSM 4582 | | | NO.: 258) | | | | | | |
| Pectobacterium | Pcs_iPDC_coSC | 44% | TQCVIIL | 3 | 5 | 1 | 1 | 1 | Potential |
| carotovorum | | | (SEQ ID | | | | | | KIVD |
| subsp. | | | NO.: 259) | | | | | | |
| Serratia | Ssp_AS12_PDC_coSC | 43% | TQCVIVI | 3 | 4 | 1 | 1 | 1 | Potential |
| sp. AS12 | | | (SEQ ID | | | | | | KIVD |
| | | | NO.: 260) | | | | | | |
| Erwinia | Ep_Ep1/96_iPDC_coSC | 40% | SQAVIVL | 4 | 5 | 2 | 1 | 0 | Potential |
| pyrifoliae | | | (SEQ ID | | | | | | KIVD |
| Ep1/96 | | | NO.: 261) | | | | | | |
| Klebsiella | Kp_342_iPDC_coSC | 41% | TQAVIVL | 3 | 6 | 2 | 1 | 1 | IPDC |
| pneumoniae | | | (SEQ ID | | | | | | |
| 342 | | | NO.: 262) | | | | | | |
| Serratia | S_TPP-BDP_coSC | 38% | SNGII | 2 | 1 | 2 | 2 | 0 | Unknown |
| odorifera | | | (SEQ ID | | | | | | |
| 4Rx13 | | | NO.: 263) | | | | | | |
| Acinetobacter | Ab_ATCC19606_iPDC_coSC | 37% | VVNIIFI | 1 | 1 | 2 | 2 | 1 | Unbranched |
| baumannii | | | (SEQ ID | | | | | | |
| | | | NO.: 264) | | | | | | |
| Francisella | F_PDC_AkaDC_coSC | 36% | FTSIL | 0 | 0 | 3 | 2 | 0 | Unbranched |
| novicida | | | (SEQ ID | | | | | | |
| | | | NO.: 265) | | | | | | |
| Schizosaccharomyces | S_Scp_PDC_coSC | 39% | FTNIIQT | 1 | 1 | 6 | 3 | 0 | Non- |
| pombe | | | (SEQ ID | | | | | | specific |
| | | | NO.: 266) | | | | | | PDC |
| Acetobacter | Ap_PDC_coSC | 32% | YTWIIWV | 1 | 1 | 3 | 7 | 0 | Specific |
| pasteurianus | | | (SEQ ID | | | | | | PDC |
| | | | NO.: 267) | | | | | | |

Table 4 summarizes the results of experiments conducted in Example 3. The data suggests that decarboxylase homologs with a higher degree of identity score to the identified KIVD substrate specificity motif tend to favor more KIV and less PDC substrate specificity, although this correlation does not necessarily extend to increased KIVD activity. Of the five sequences classified as KIVD, all five had KIV/pyruvate activity ratios about 40. Of the five sequences classified as potential KIVD, two had KIV/pyruvate ratios>50, two others had KIV/pyruvate ratios>20, and the other had a modest preference for KIV.

Thus, the effect of the specificity motif imparts greater effects on substrate specificity (see bolded column highlighting KIV/Pyruvate Activity Ratio) and less on influencing KIVD specific activity. Accordingly, factors independent of the substrate specificity motif may also contribute to the amount of KIVD activity.

### Example 4

### Identification of Specificity Motif from Francisella Decarboxylase

A surprising result from the experiments performed in Example 3 was the favorable KIV/pyruvate ratio for the decarboxylase derived from Francisella cf. novicida 3523. This decarboxylase candidate had been classified as an “unbranched” decarboxylase, due to the use of several residues hypothesized to preclude activity for bulky branched substrates such as KIV. Specifically, the F. novicida decarboxylase favors KIV over pyruvate without using the same motif employed by other variants. Notably, it comprises F286, T377, and 1461 based on numbering from the L. lactis KivD—thus, the positioning of KIV was hypothesized to be restricted by the bulk of F286, the beta branching methyl of T377, and the additional methyl of 1461.

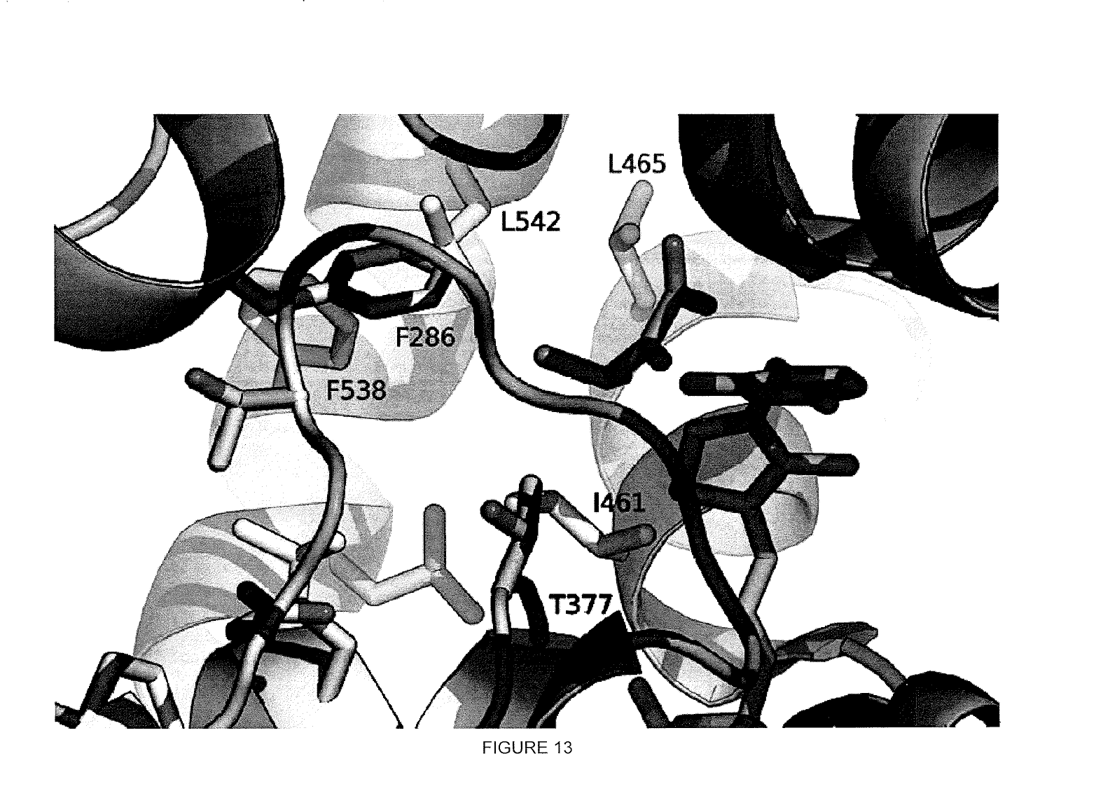

In this example, a partial model for Francisella cf. novicida 3523 decarboxylase was created by modeling mutations onto the structure of the L. lactis KdcA (2vbf). To approximate the KIV position, a KIV molecule was modeled using SHARPEN/OpenBabel to create the coordinates and PyMOL to adjust the torsions. The substrate was placed in accord with the observed ligand positions in 2vk1 and 2vbg, corresponding to structures from S. cerevisiae (PDC) and L. lactis (KdcA), respectively (FIG. 13).

TABLE 4
Profile of KIV and Pyruvate specific activity and KIV/pyruvate specific activity
ratio for decarboxylase homologs expressed in GEVO4001. Error bars for specific
activity values represent combined errors from two measurements. Error bars for
the specific activity ratios represent combined errors from two measurements.

| | | Spe- | KIV | Pyruvate | KIV/ | Pyruvate/ | Expression |
|---|---|---|---|---|---|---|---|
| | | cificity | “Specific” | “Specific” | Pyruvate | KIV | Relative to |
| | | amino | Activity | Activity | Activity | Activity | LI_KdcA |
| GENE | ID% | acids | (U/mg) | (U/mg) | Ratio | Ratio | (set to 100) |
| LI_KdcA_ | 88 | SQFVIMF | 17.6 ± 0.69 | 0.19 ± 0.1 | 91.2 ± 9.1 | 0.01 ± 0 | 100 ± 0 |
| coSC | | (SEQ ID | | | | | |
| | | NO.: 252) | | | | | |
| Se_p-iPDC_ | 47 | SQFVIIF | 1.2 ± 0.02 | 0.01 ± 0 | 93 ± 13.79 | 0.01 ± 0 | 60.8 ± 20.1 |
| coSC | | (SEQ ID | | | | | |
| | | NO.: 253) | | | | | |
| Mc_iPDCh_ | 47 | SQFVIIF | 9.1 ± 0.29 | 0.11 ± 0 | 77.4 ± 3.86 | 0.01 ± 0 | 100.7 ± 41.5 |
| coSC | | (SEQ ID | | | | | |
| | | NO.: 254) | | | | | |
| Bm_QM-B1551_ | 46 | SQFVILF | 0.4 ± 0.02 | 0 | 57.4 ± 15.75 | 0.01 ± 0 | 55.1 ± 21.3 |
| iPDC_coSC | | (SEQ ID | | | | | |
| | | NO.: 255) | | | | | |
| Ss_iPDC_coSC | 46 | SQFVIIF | 0.8 ± 0.02 | 0.01 ± 0 | 46.6 ± 8.6 | 0.02 ± 0 | 81.1 ± 33.9 |
| | | (SEQ ID | | | | | |
| | | NO.: 256) | | | | | |
| Bc_BDRD-ST24_ | 47 | TQFVILF | 1.9 ± 0.14 | 0.07 ± 0 | 27.8 ± 3.42 | 0.03 ± 0 | 133 ± 27.6 |
| iPDC_coSC | | (SEQ ID | | | | | |
| | | NO.: 257) | | | | | |
| So_DSM-4582_ | 44 | TQSVIVI | 9.7 ± 0.65 | 0.16 ± 0 | 59.2 ± 7.6 | 0.01 ± 0 | 118.1 ± 51.2 |
| iPDC_coSC | | (SEQ ID | | | | | |
| | | NO.: 258) | | | | | |
| Pcs_iPDC_ | 44 | TQCVIIL | 0.9 ± 0.11 | 0.03 ± 0 | 26.6 ± 5.19 | 0.03 ± 0 | 61 ± 7.2 |
| coSC | | (SEQ ID | | | | | |
| | | NO.: 259) | | | | | |
| Ssp_AS12_ | 43 | TQCVIVI | 5.8 ± 0.38 | 0.09 ± 0 | 61.6 ± 7.72 | 0.01 ± 0 | 152.8 ± 41.6 |
| PDC_coSC | | (SEQ ID | | | | | |
| | | NO.: 260) | | | | | |
| Ep_Ep1/96_ | 40 | SQAVIVL | 0 | 0 | 13.3 ± 21.9 | 0.15 ± 0.26 | 79.6 ± 3 |
| iPDC_coSC | | (SEQ ID | | | | | |
| | | NO.: 261) | | | | | |
| Kp_342_iPDC_ | 41 | TQAVIVL | 0.6 ± 0.03 | 0.02 ± 0 | 31.5 ± 6.8 | 0.03 ± 0 | 115 ± 21.2 |
| coSC | | (SEQ ID | | | | | |
| | | NO.: 262) | | | | | |
| S_TPP-BDP_ | 38 | SNGII | 0 | 0.04 ± 0 | 0.33 ± 0.10 | 4.6 ± 4.2 | 63.9 ± 32 |
| coSC | | (SEQ ID | | | | | |
| | | NO.: 263) | | | | | |
| Ab_ATCC19606_ | 37 | VVNIIFI | 0 | 0.05 ± 0 | 0.16 ± 0.23 | 6.03 ± 12.2 | 35.2 ± 26.3 |
| iPDC_coSC | | (SEQ ID | | | | | |
| | | NO.: 264) | | | | | |
| F_PDC_AkaDC_ | 36 | FTSIL | 6.7 ± 0.42 | 0.6 ± 0.18 | 14.82 ± 1.9 | 0.11 ± 0.0 | 80 ± 29.7 |
| coSC | | (SEQ ID | | | | | |
| | | NO.: 265) | | | | | |
| S_Scp_PDC_ | 39 | FTNIIQT | 0 | 0.41 ± 0.02 | 0 ± 0.0 | 19.2 ± 5.41 | 15.8 ± 22.3 |
| coSC | | (SEQ ID | | | | | |
| | | NO.: 266) | | | | | |
| Ap_PDC_coSC | 32 | YTWIIWV | 0 | 7.6 ± 0.30 | 0 ± 0.0 | 798.5 ± 259.31 | 74.5 ± 43.4 |
| | | (SEQ ID | | | | | |
| | | NO.: 267) | | | | | |
| GEVO4001 | n.a. | n.a. | 0 | 0 | 0% | 0 | 0 |
| (empty | | | | | | | |
| strain) | | | | | | | |

A sequence alignment between the L. lactis KivD and the Francisella decarboxylase allows for the identification of a separate motif capable of conferring KIV/pyruvate specificity, “FTSILFL” (SEQ ID NO: 240), corresponding to residues F305, T397, S401, I481, L485, F556, and L560 of the Francisella cf. novicida 3523 decarboxylase of SEQ ID NO: 198. Further analysis revealed that KIV can still be favored over pyruvate because the L485 residue has the flexibility to get out the way of KIV steric bulk, also creating space at the “top” of the active site (see FIG. 13). Characterization of the separate KIV/pyruvate specificity motif allowed for the identification of several additional decarboxylases harboring desired KIV/pyruvate specificity (see SEQ ID NOs: 199-214).

### Example 5

### Generation of Mutant PDC to Efficiently Catalyze Conversion of α-Ketoisovalerate to Isobutyraldehyde

This example shows how a mutant PDC can be generated which efficiently catalyzes the conversion of KIV to isobutyraldehyde.

This example was generated based upon (1) a visual inspection of the L. lactis branched-chain KdcA (LI_KDCA) structure (2vbf) and comparison of that structure with high-resolution models of the yeast PDC structure (2vk1 and 2vk8); (2) analysis of the experimentally observed KIV/pyruvate activity ratio described above in examples 3-4, and (3) protein modeling and design calculations that assessed the detailed energetic consequences that result from a panel of mutations to PDC.

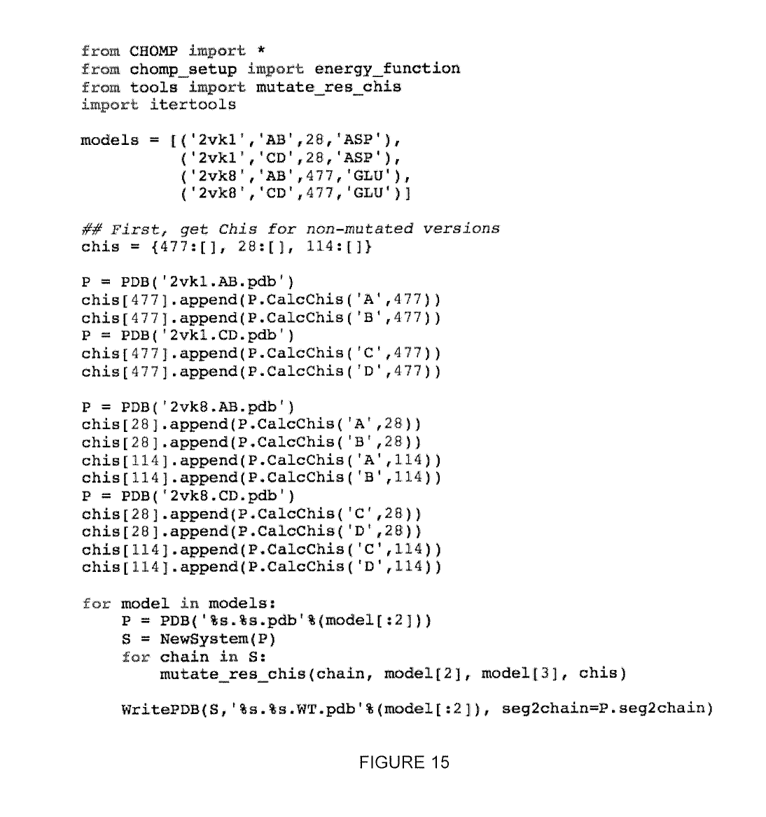

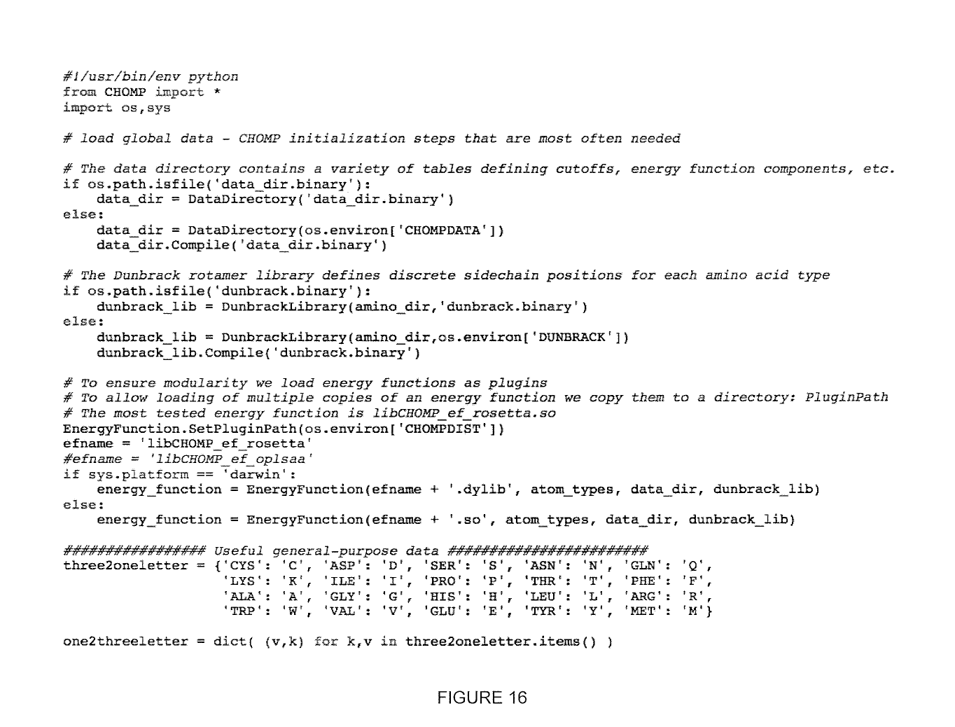

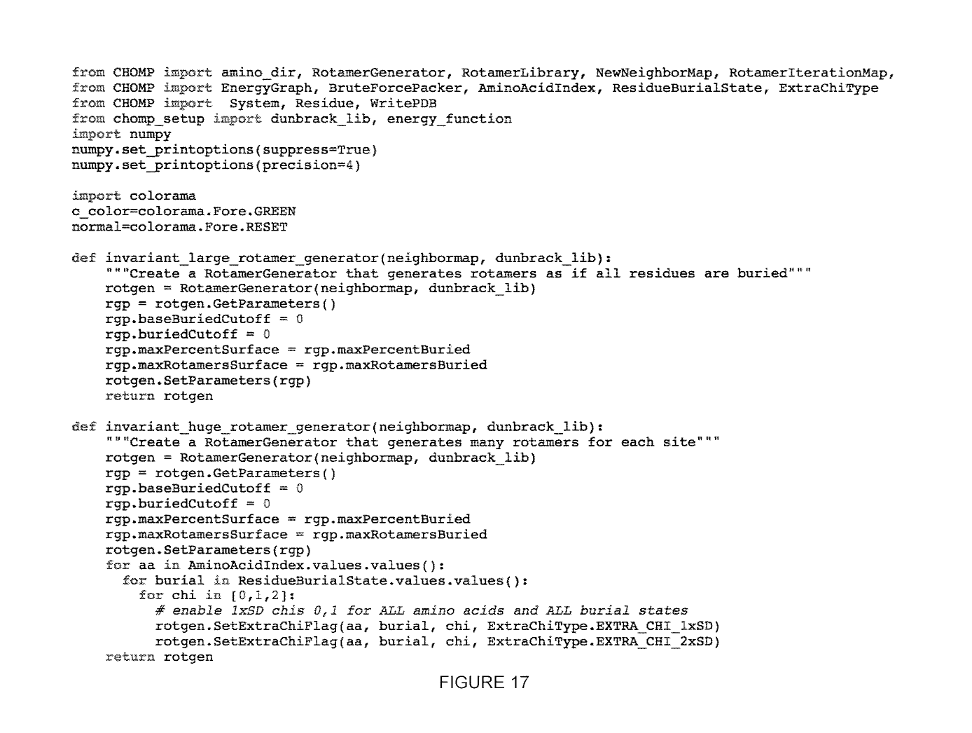

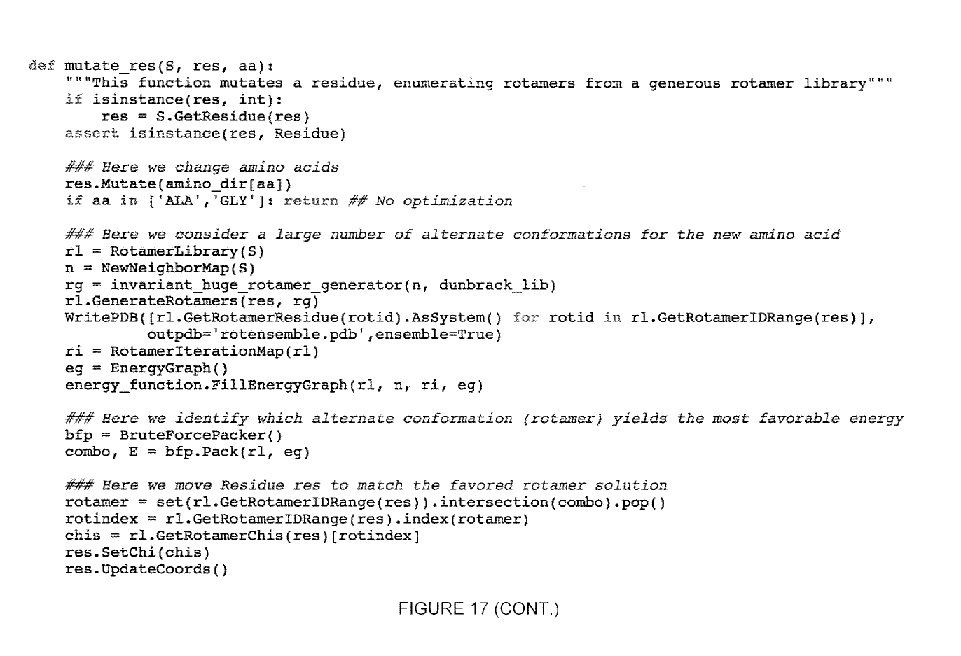

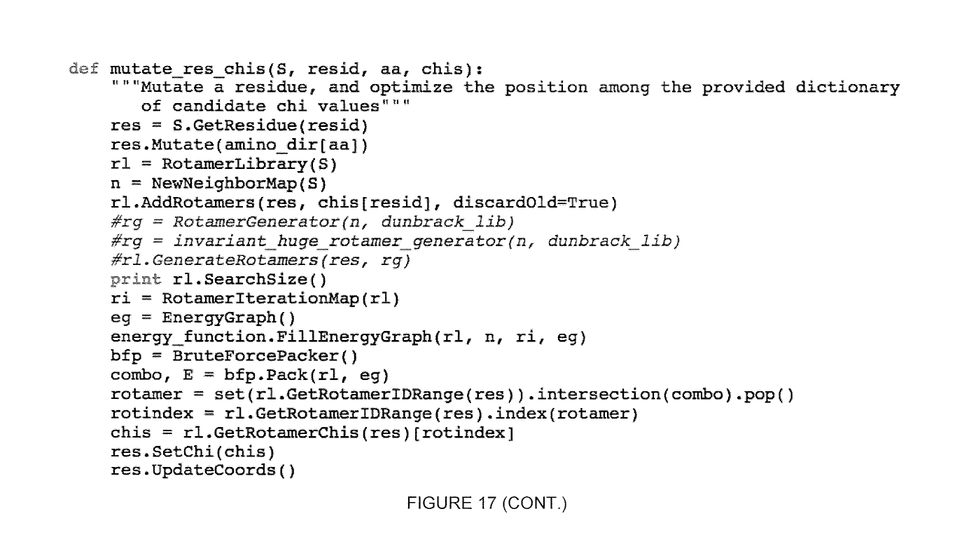

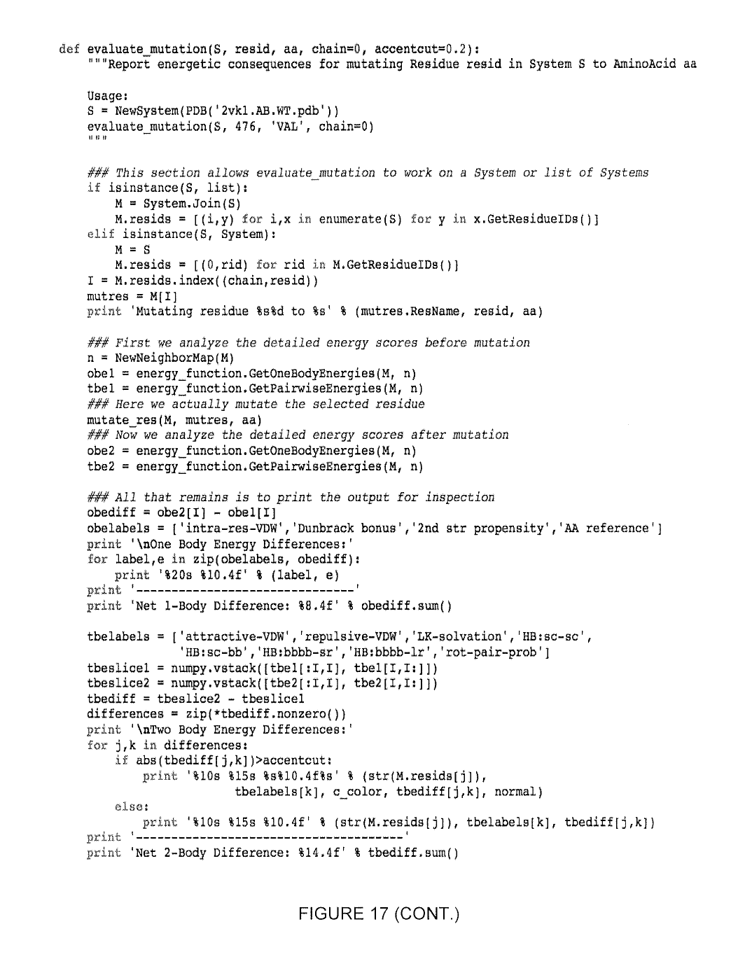

Briefly, eight models for the wild-type yeast S. cerevisiae PDC1 (SEQ ID NO: 241) active site were obtained. Each pdb file (2vk1 and 2vk8) has four chains, with two active sites for the A/B dimer and two active sites for the C/D dimer. To convert these wild-type models, mutations were reverted to capture the active enzyme. Specifically, 2vk8 E477Q and 2vk1 A28D were converted. These models were prepared using the SHARPEN protein modeling library (Loksha et al., 2009, J. Comput. Chem. 30(6): 999-1005). SHARPEN is an open-source library rather than a standalone executable program; custom modeling tasks are performed by writing relatively short Python scripts. The first such script (FIG. 15) was used to generate models for wild-type S. cerevisiae PDC1 given several crystal structures for point mutations thereof. Subsidiary code is included in FIGS. 16 and 17.

Next, additional software was generated to use the SHARPEN protein modeling library to prospectively model individual mutations of interest and to decompose the resulting energy difference into component energy terms using an implementation of the all-atom Rosetta energy model (Rohl et al., 2004 Methods Enzymol. 383:66-93). The Rosetta energy model considers several physical terms: (i) van der Waals energy, (ii) Lazaridis-Karplus solvation energy, and (iii) hydrogen bonding energy. The energy model also includes several statistical, knowledge-based terms: (iv) a coarse-grained term that favors or penalizes the proximity of amino-acid centroids, (v) a term that favors sidechain conformations similar to canonical rotamers, (vi) a secondary structure propensity term that favors specific amino acids as a function of φ and ψ and, (vii) an amino-acid dependent reference energy. This energy function can catch unfavorable interactions that might not be properly assessed during a visual inspection of a protein model. Accordingly, prospective calculations that predict the detailed energetic consequences of mutations complement visual analysis.

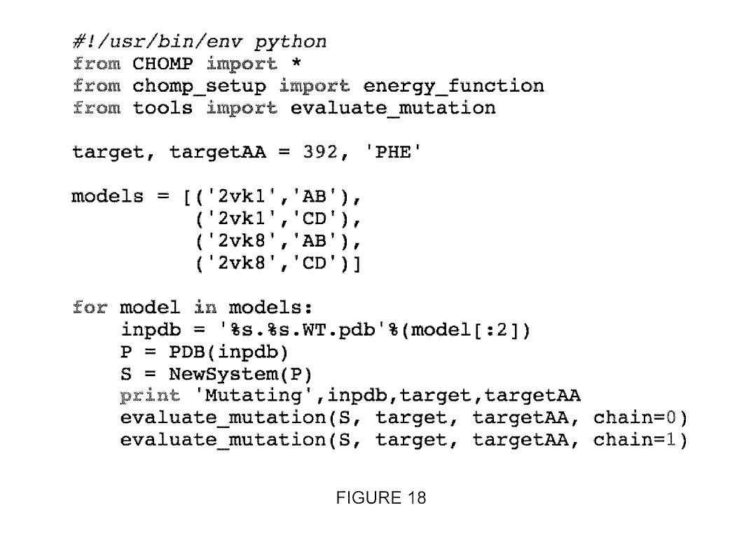

To assess mutations in detail, models for the mutants were generated, allowing the mutated sidechains to select new conformations from an expanded Dunbrack rotamer library (FIG. 18). Models for a variety of mutations were calculated, including (a): I476V, (b): T388Q, (c): F292S, (d): A392F, (e): S408G, (f): A392F and S408G, (g): A392F, S408G, and V410D, (h): T556F, and (i): Q552M, wherein the mutations are relative to the S. cerevisiae PDC1 of SEQ ID NO: 241. To determine if these mutations were likely to be compatible with the remainder of S. cerevisiae PDC1 (SEQ ID NO: 241), we compared the Rosetta energy before and after the mutations, inspecting the individual components of the energy function to best understand the nature of the predicted energy shift. This detailed analysis proved useful to interpret the results of subsequent calculations in which multiple mutations were simultaneously introduced into our structural models for S. cerevisiae PDC1 (SEQ ID NO: 241).

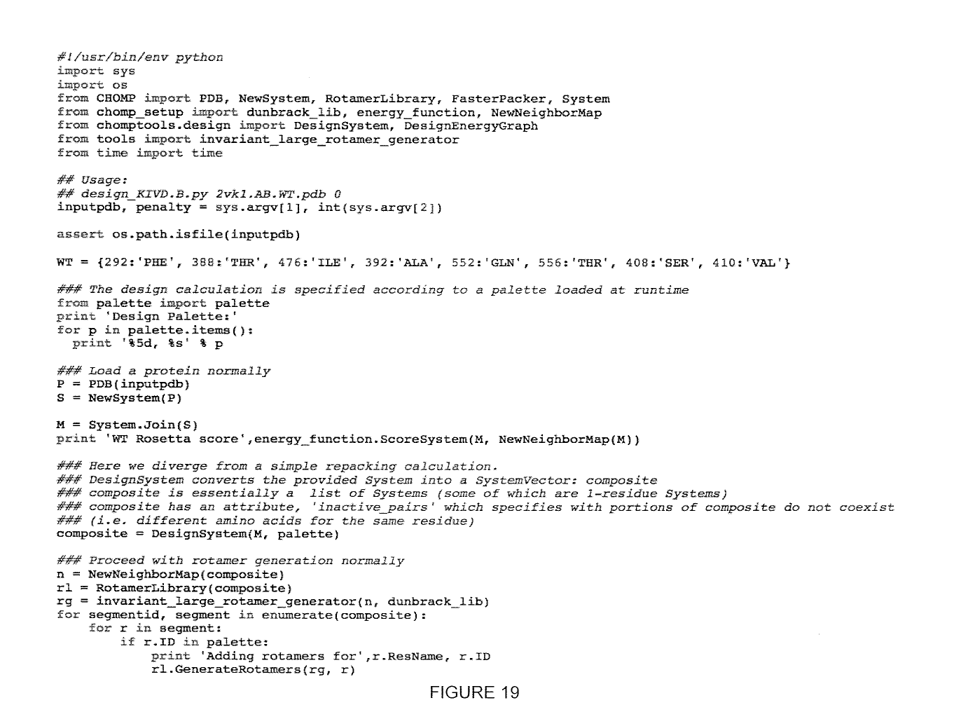

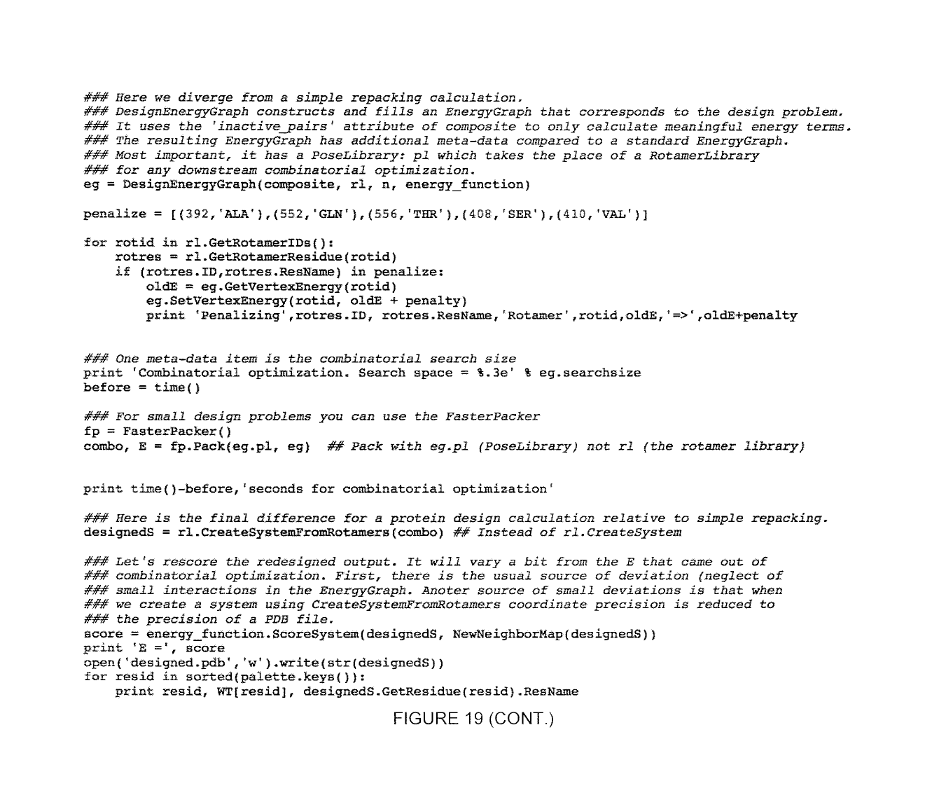

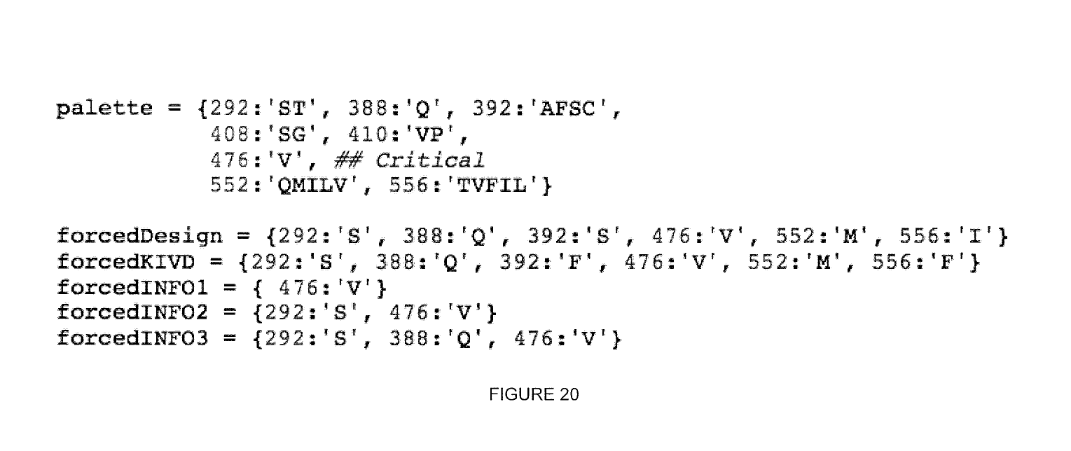

After inspecting individual mutations, we turned to the larger problem of predicting the structure and Rosetta score of variants with multiple mutations. For each initial S. cerevisiae PDC1 wild-type model calculated above (SEQ ID NO: 241), a protein design calculation (FIG. 19) identified the sequence and the rotamer sidechain positions for that sequence which minimize the energy according to the all-atom Rosetta energy model. The sidechain combinatorial optimization used the FASTER algorithm as implemented in SHARPEN (Loksha et al., 2009, J. Comput. Chem. 30(6): 999-1005). Eight design positions were chosen as illustrated in Table 5. The choices were selected to encompass wild-type yeast PDC (*) or to match amino acids found in decarboxylases observed to exhibit a KIV/pyruvate activity ratio of >10, including (a): 292, Ser or Thr; (b): 388, Gln; (c): 392, Ala*, Ser, Cys, or Phe; (d) 408: Ser* or Gly; (e): 410: Val* or Pro; (f): 476: Val; (g): 552: Gln*, Met, Ile, Leu, or Val; and (h): 556: Thr*, Val, Phe, Ile, or Leu. Together these design alternatives comprise 2×4×2×2×5×5 combinations, a sequence space of 800 members (FIG. 20). The resulting calculations are shown in the redesign” column in Table 5. Beyond the enforced changes T388Q and I476V, redesign resulted in 1-2 additional mutations. To identify additional acceptable mutations, protein design calculations were repeated as described above, but with a penalty applied to disfavor solutions that retained the wild-type PDC amino acids. By increasing the penalty, and redesigning, sets of amino acids found in homologs with favorable KIV to pyruvate ratios that are likely to be compatible with existing PDC structure were identified.

The combined modeling analysis allowed for the determination that critical mutations of F292S, T388Q, and I476V are tolerable in the context of the yeast PDC structure, wherein the F292S, T388Q, and I476V mutations are relative to the S. cerevisiae PDC1 of SEQ ID NO: 241 and correspond with positions S286, Q377, and V461 of the L. lactis KivD (SEQ ID NO: 197). Modeling was also a useful filter to determine that candidate mutations at positions A392 (A392F) and T556 (T556F) result in steric clashes. Specifically, A392F leads to a clash with S408 and V410, while T556F results in a steric clash with D38, H114, D291, F292, Q552, and N560. Fortunately, however, the known favorable KIV/pyruvate activity of decarboxylase enzymes (Table 4) suggests alternate amino acids for residues 392 (Ser, Cys, Phe) and 556 (Val, Phe, Ile, Leu).

As observed in the design calculations, alternatives to phenylalanine at positions A392 and T556 could be incorporated into the PDC structure. An additional mutation at Q552 was also determined to confer beneficial properties. In sum, S. cerevisiae PDC1 harboring at least one of eight mutations at positions corresponding to the F292, T388, A392, S408, V410, 1476, Q552, and T556 positions of the S. cerevisiae PDC1 can be made to improve specificity for KIV.

Although the final design incorporates six mutations into the S. cerevisiae PDC1, the enzyme is virtually identical to the wild-type in terms of energy score (score of −1743 Rosetta energy units in the mutant enzyme versus a score of −1746 Rosetta energy units in the wild-type PDC enzyme).

TABLE 5
Summary of computational protein design calculations conducted on 4
different structural models (2vk1.AB, 2vk1.CD, 2vk8.CD, 2vk8.AB).

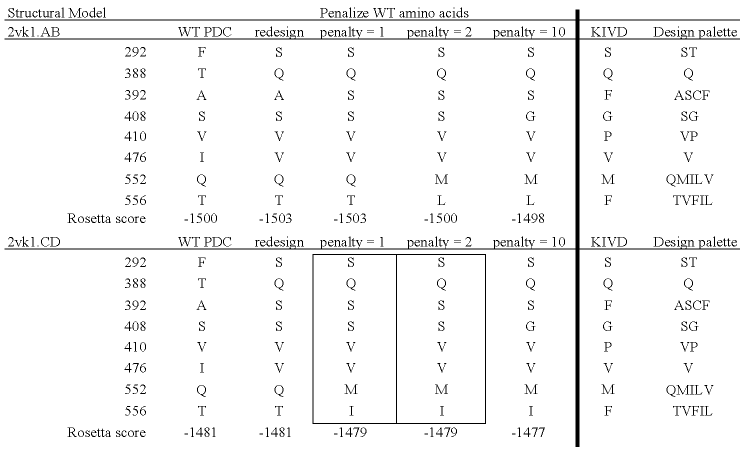

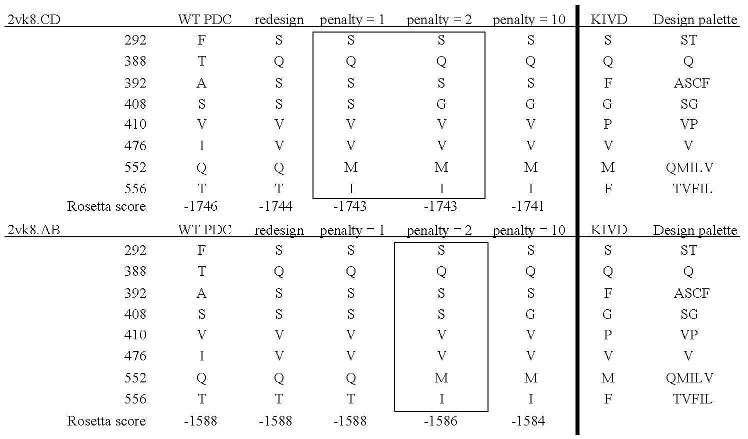

Last column indicates which residues were allowed at each position. KIVD column and “WT PDC” column indicate, respectively, which residue is adopted by the wild-type KIVD and PDC.
Shaded cells indicate amino acids other than wild-type PDC.
Boxed designs correspond to the final design (SEQ ID NOS.: 268-270).
A standard protein design calculation results in amino acid choices shown in “redesign” column.
Penalties disfavoring the wild-type PDC residue (a penalty of 1 or 2 Rosetta eu) resulted in desirable sequence.

The foregoing detailed description has been given for clearness of understanding only and no unnecessary limitations should be understood there from as modifications will be obvious to those skilled in the art.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

The disclosures, including the claims, figures and/or drawings, of each and every patent, patent application, and publication cited herein are hereby incorporated herein by reference in their entireties.

1.-101. (canceled)

102. A recombinant microorganism comprising at least one nucleic acid molecule encoding a modified decarboxylase enzyme, wherein the modified decarboxylase enzyme has one or more modifications or mutations at positions corresponding to amino acids selected from: (a) serine 286 of the L. lactis KIVD (SEQ ID NO: 197); (b) glutamine 377 of the L. lactis KIVD (SEQ ID NO: 197); (c) phenylalanine 381 of the L. lactis KIVD (SEQ ID NO: 197); (d) valine 461 of the L. lactis KIVD (SEQ ID NO: 197); (e) isoleucine 465 of the L. lactis KIVD (SEQ ID NO: 197); (f) methionine 538 of the L. lactis KIVD (SEQ ID NO: 197); and (g) phenylalanine 542 of the L. lactis KIVD (SEQ ID NO: 197).

103. The recombinant microorganism of claim 102, wherein the residue corresponding to position 286 of the L. lactis KIVD (SEQ ID NO: 197) is replaced with a residue selected from serine, threonine, asparagine, glycine, alanine, proline, glutamine, and aspartic acid.

104. The recombinant microorganism of claim 102, wherein the residue corresponding to position 377 of the L. lactis KIVD (SEQ ID NO: 197) is replaced with a residue selected from glutamine, serine, threonine, and asparagine.

105. The recombinant microorganism of claim 102, wherein the residue corresponding to position 381 of the L. lactis KIVD (SEQ ID NO: 197) is replaced with a residue selected from phenylalanine, alanine, isoleucine, leucine, methionine, tryptophan, tyrosine, and valine.

106. The recombinant microorganism of claim 102, wherein the residue corresponding to position 461 of the L. lactis KIVD (SEQ ID NO: 197) is replaced with a residue selected from valine, phenylalanine, alanine, isoleucine, leucine, methionine, tryptophan, and tyrosine.

107. The recombinant microorganism of claim 102, wherein the residue corresponding to position 465 of the L. lactis KIVD (SEQ ID NO: 197) is replaced with a residue selected from isoleucine, valine, phenylalanine, alanine, leucine, methionine, tryptophan, and tyrosine.

108. The recombinant microorganism of claim 102, wherein the residue corresponding to position 538 of the L. lactis KIVD (SEQ ID NO: 197) is replaced with a residue selected from methionine, isoleucine, leucine, valine, alanine, cysteine, glycine, phenylalanine, proline, tryptophan, and tyrosine.

109. The recombinant microorganism of claim 102, wherein the residue corresponding to position 542 of the L. lactis KIVD (SEQ ID NO: 197) is replaced with a residue selected from phenylalanine, isoleucine, leucine, methionine, valine, alanine, cysteine, glycine, proline, tryptophan, and tyrosine.

110.-122. (canceled)

123. The recombinant microorganism of claim 102, wherein the modified decarboxylase enzyme is derived from a corresponding unmodified decarboxylase enzyme selected from SEQ ID NOs 1-214.

124.-137. (canceled)

138. The recombinant microorganism of claim 102, wherein the modified decarboxylase enzyme is derived from a corresponding unmodified decarboxylase enzyme selected from the group consisting of PDC1 (SEQ ID NO: 241), PDC5 (SEQ ID NO: 242), and PDC6 (SEQ ID NO: 243) of Saccharomyces cerevisiae.

139. (canceled)

140. The recombinant microorganism of claim 102, wherein the recombinant microorganism comprises a deletion or disruption of one or more endogenous pyruvate decarboxylase genes.

141. The recombinant microorganism of claim 102, wherein the recombinant microorganism comprises an isobutanol producing metabolic pathway comprising one or more isobutanol metabolic pathway enzymes selected from acetolactate synthase, ketol-acid reductoisomerase, dihydroxy acid dehydratase, and alcohol dehydrogenase.

142. The recombinant microorganism of claim 141, wherein the recombinant microorganism comprises a ketol-acid reductoisomerase and the ketol-acid reductoisomerase is an NADH-dependent ketol-acid reductoisomerase (NKR).

143. The recombinant microorganism of claim 141, wherein the recombinant microorganism comprises an alcohol dehydrogenase and the alcohol dehydrogenase is an NADH-dependent alcohol dehydrogenase.

144. The recombinant microorganism of claim 102, wherein the recombinant microorganism comprises a metabolic pathway for the production of a metabolite selected from 1-propanol, 1-butanol, 2-methyl-1-butanol, 3-methyl-1-butanol, and 2-phenylethanol.

145. The recombinant microorganism of claim 102, wherein the recombinant microorganism is a yeast microorganism.

146. The recombinant microorganism of claim 102, wherein the recombinant microorganism is a prokaryotic microorganism.

147. A method of producing isobutanol, comprising:
(a) providing a recombinant microorganism of claim 141; and
(b) cultivating the recombinant microorganism in a culture medium containing a feedstock providing a carbon source until the isobutanol is produced.

148.-150. (canceled)

151. An isolated nucleic acid molecule encoding a modified decarboxylase enzyme, wherein the modified decarboxylase enzyme has one or more modifications or mutations at positions corresponding to amino acids selected from: (a) serine 286 of the L. lactis KIVD (SEQ ID NO: 197); (b) glutamine 377 of the L. lactis KIVD (SEQ ID NO: 197); (c) phenylalanine 381 of the L. lactis KIVD (SEQ ID NO: 197); (d) valine 461 of the L. lactis KIVD (SEQ ID NO: 197); (e) isoleucine 465 of the L. lactis KIVD (SEQ ID NO: 197); (f) methionine 538 of the L. lactis KIVD (SEQ ID NO: 197); and (g) phenylalanine 542 of the L. lactis KIVD (SEQ ID NO: 197).

152.-154. (canceled)
